(19) United States
(12) Patent Application Publication
(10) Pub. No.: US 2022/0117989 A1
(43) Pub. Date: Apr. 21, 2022

(54) COMPOSITIONS AND METHODS FOR INDUCING OR SUPPLEMENTING SOCS3 TO ABROGATE TUMOR GROWTH AND PROLIFERATIVE RETINOPATHY

(71) Applicant: Children's Medical Center Corporation, Boston, MA (US)

(72) Inventors: Lois Smith, West Newton, MA (US); Andreas Stahl, Boston, MA (US); Jean-Sebastien Joyal, Boston, MA (US); Ye Sun, Chestnut Hill, MA (US)

(21) Appl. No.: 17/422,638

(22) PCT Filed: Jan. 17, 2020
(86) PCT No.: PCT/US2020/014046

§ 371 Date: Jul. 13, 2021

Related U.S. Application Data
(60) Provisional application No. 62/794,507, filed on Jan. 18, 2019.

(51) Int. Cl. A61K 31/7048; A61P 35/00

(52) U.S. Cl. CPC A61K 31/7048; A61P 35/00

(57) ABSTRACT

The invention provides compositions and methods for inhibiting tumor growth by up-regulating and/or supplementing SOCS3 in tumor and/or tumor-associated tissues of a subject or in cells in vitro. Compounds capable of up-regulating SOCS3, including flavanones, or otherwise of up-regulating the ACh pathway, e.g., at the NMJ, are identified and provided, as are methods for identifying additional agents as inducers of SOCS3.

### CROSS REFERENCE TO RELATED APPLICATIONS

This application claims the benefit of U.S. Provisional Application 62/794,507 filed on Jan. 18, 2019. The entire contents of this application is incorporated herein by reference in its entirety.

### TECHNICAL FIELD

The present disclosure relates in general to compositions for the treatment of cancer and, in particular, to agents which modulate expression or activity of suppressor of cytokine signaling (SOCS3) molecules.

### BACKGROUND

Invasive cancer is the leading cause of death in the developed world, accounting for 13% of all deaths each year. Due to aging populations, global cancer rates have been increasing. Current therapies have targeted intrinsic properties of tumors, but have paid less attention to the role of surrounding tissue in promoting or inhibiting tumor growth.

Inflammatory cytokines and growth factors independently drive angiogenesis. However, their integrated role in pathologic and physiologic angiogenesis is not fully understood. Physiologic angiogenesis is tightly regulated. In contrast, there is excessive and disorganized growth of pathologic blood vessels in diseases such as proliferative retinopathy and cancer.

Suppressors of cytokine signaling (SOCS) are known negative feedback regulators of inflammation and growth factor signaling (Starr 1997, Wang 2002). SOCS3 is transiently induced by inflammatory mediators such as LPS, IL-6 and TNFα (Lebel, 2000, Jiang 2012). SOCS3 inhibits cytoplasmic effectors like JAK/STAT kinases and deactivates tyrosine kinase receptor signaling, including IGF-1 receptor (Dey, 2000). It also regulates endothelial cell (EC) apoptosis.

### SUMMARY

The present disclosure derives, at least in part, from the hypothesis that endogenous angiostatic regulators exist to restrain pathologic vascular growth triggered by massive inflammatory and growth factor angiogenic stimuli. Here, SOCS3 has been identified as an endogenous angiostatic regulator and tumor suppressor that can be harnessed pharmacologically to prevent pathologic vessel and tumor growth. This is the first identification of a role for SOCS3 in regulating angiogenesis and tumor suppression in vivo.

The present disclosure provides compositions and methods comprising or related to up-regulating SOCS3 in mammalian cells, either in vitro or as therapeutics.

In one aspect, the disclosure provides a method of inhibiting pathological blood vessel growth involving administering to a subject with pathological blood vessel growth a modified suppressor of cytokine signaling (SOCS3) fusion protein or a vector expressing a SOCS3 polypeptide, thereby inhibiting pathological blood vessel growth in the subject.

In another aspect, a method of treating cancer comprises administering a therapeutically effective amount of a Socs3 peptidomimetic.

In another aspect, a method of treating cancer comprises administering an effective amount of a modified suppressor of cytokine signaling (SOCS3) fusion protein, a vector expressing a SOCS3 polypeptide, a Socs3 polypeptide or active fragments thereof, a peptidomimetic or combinations thereof, and a checkpoint inhibitor. The administration of the Socs3 molecules can be in combination with the checkpoint inhibitor or at alternative times and routes.

Another aspect of the disclosure provides a method of inhibiting pathological blood vessel growth involving administering a flavanone to a subject with pathological blood vessel growth, thereby inhibiting pathological blood vessel growth.

An additional aspect of the disclosure provides a method for inhibiting the growth of a tumor in a subject involving administering a flavanone to the subject in an amount sufficient to increase the level of SOCS3 in the tumor, in a tumor-associated tissue, or in the general host tissue of the subject, thereby inhibiting the growth of the tumor in the subject.

A further aspect of the disclosure provides a method for inhibiting the growth of a tumor in a subject involving administering a modified SOCS3 fusion protein or a vector that expresses a modified SOCS3 fusion protein to the subject in an amount sufficient to increase the level of SOCS3 in the tumor, in a tumor-associated tissue, or host tissue of the subject, thereby inhibiting the growth of the tumor in the subject.

In one embodiment, the flavanone is Butin, Eriodictyol, Hesperetin, Hesperidin, Homoeriodictyol, Isosakuranetin, Naringenin, Naringin, Pinocembrin, Poncirin, Sakuranetin, Sakuranin and/or Sterubin.

Optionally, the tumor is a solid tumor, optionally a melanoma, a lung cancer, a gastric cancer, a liver cancer, a colon cancer, an esophageal cancer, and/or a pancreatic cancer.

In certain embodiments, the tumor-associated tissue is a host tumor bed, vascular tissue, neural tissue, or muscle tissue, or any combination thereof. Optionally, the host tissue or general host tissue is stromal tissue of the subject.

In one embodiment, the vector that expresses a modified SOCS3 fusion protein is a viral vector, optionally an AAV or lentiviral vector. In certain embodiments, the vector is an adenoviral vector, optionally an AAV vector of AAV-1 to AAV-9 (and/or hybrid forms thereof). In some embodiments, expression of the vector is tissue-specific. In certain embodiments, expression of the vector is global.

Another aspect of the disclosure provides an in vitro method for identifying a candidate inducer of SOCS3 protein expression involving making a SOCS3 reporter gene construct including a reporter gene under control of the SOCS3 promoter; introducing the SOCS3 reporter gene construct into a mammalian cell; contacting the mammalian cell with a test agent under conditions suitable for SOCS3 reporter gene expression in the mammalian cell; and comparing levels of the SOCS3 reporter gene in the mammalian cell contacted with the test agent with levels of the SOCS3 reporter gene in an appropriate control mammalian cell, where identification of elevated SOCS3 reporter gene levels in the mammalian cell contacted with the test agent identifies the test agent as a candidate inducer of SOCS3 protein expression.

In one embodiment, the mammalian cell contacted with the test agent is a carcinoma cell, a neuronal cell, an immune cell, a macrophage, a vascular cell or a muscle cell, or any combination thereof.

An additional aspect of the disclosure provides a method for inhibiting the growth of a tumor in a subject involving administering an inducer of SOCS3 protein identified by a screening method of the disclosure to the subject in an amount sufficient to increase the level of SOCS3 in the tumor or in a tumor-associated tissue of the subject, thereby inhibiting the growth of the tumor in the subject.

The disclosure also provides as another aspect a composition including a modified suppressor of cytokine signaling (SOCS3) protein.

In certain embodiments, the protein is active intracellularly. Optionally, the modified SOCS3 protein is fused to an antibody, or fragment thereof. In one embodiment, the antibody, or fragment thereof, is a single chain antibody (scFv). Optionally, the scFv is a cell-internalizing scFv. In certain embodiments, the scFv is internalized in pathologic blood vessels, tumor associated cells and/or neurons. In some embodiments, the antibody, or fragment thereof, is a single domain antibody (sdAb). Optionally, the sdAb is bispecific.

In some embodiments, the modified SOCS3 protein is fused to a cell-penetrating peptide.

In certain embodiments, a composition of the disclosure further includes one or more molecules to increase the half-life.

Another aspect of the disclosure provides a fusion protein including a modified SOCS3 protein fused to at least one scFv.

In one embodiment, the scFv is a cell-internalizing scFv. Optionally, the scFv is internalized in pathologic blood vessels or neurons.

An additional aspect of the disclosure provides a fusion protein including a modified SOCS3 protein fused to at least one sdAb.

A further aspect of the disclosure provides a fusion protein that includes a modified SOCS3 protein fused to at least one cell-penetrating peptide.

In one embodiment, the fusion protein further includes one or more molecules to increase half-life.

Another aspect of the disclosure provides a method of treating an autoimmune disease or sepsis involving administering to a subject with an autoimmune disease or sepsis a composition that includes a flavanone, a candidate inducer of SOCS3 protein identified by a screening method of the disclosure, a modified SOCS3 fusion protein, or a vector that expresses a modified SOCS3 fusion protein, thereby treating the autoimmune disease or sepsis.

In one embodiment, the autoimmune disease is associated with pathological blood vessel growth. Optionally, the composition is administered to a host tissue.

In certain embodiments, the autoimmune disease is retinopathy. Optionally, the retinopathy is retinopathy of prematurity, diabetic retinopathy or age related macular degeneration.

In some embodiments, the autoimmune disease is juvenile rheumatoid arthritis (JRA).

In certain embodiments, the subject has sepsis.

In another aspect, the disclosure provides a method of inhibiting tumor growth involving administering to a subject with a solid tumor a composition that includes a candidate inducer of SOCS3 protein identified by a method of the disclosure, a modified SOCS3 fusion protein, a vector that expresses a SOCS3 polypeptide, and/or a flavanone thereby inhibiting tumor growth.

In one embodiment, the composition is administered to the tumor, and/or optionally to tumor-associated cells, where optionally the cells are associated with the tumors.

In certain embodiments, the composition is administered to nerve fibers. Optionally, the nerve fibers are associated with the tumors.

An additional aspect of the disclosure provides a kit that includes a modified suppressor of cytokine signaling SOCS3 protein of the disclosure or a vector that expresses a SOCS3 polypeptide, and instructions for its use in inhibiting pathological blood vessel growth. pathological neovascularization and/or tumor growth.

Another aspect of the disclosure provides a method of inhibiting or decreasing solid tumor in a subject, involving administering an effective amount of SOCS3, a vector that expresses a SOCS3 polypeptide and/or a SOCS3 inducing agent to a tumor-associated tissue in the subject and/or to a host tissue of the subject, such that growth of the solid tumor is inhibited or decreased.

An additional aspect of the invention provides a method of inhibiting or decreasing solid tumor growth in a subject, involving selecting a subject having a solid growth tumor and systemically administering an effective amount of SOCS3, a vector that expresses a SOCS3 polypeptide, and/or a SOCS3 inducing agent to the subject, such that growth of the solid tumor is inhibited or decreased.

In one embodiment, the solid tumor is a lung carcinoma, a glioblastoma, a gastric adenocarcinoma, a hepatocellcular carcinoma or a melanoma.

In certain embodiments, the method further involves delivering the SOCS3 and/or the SOCS3 inducing agent directly to the solid tumor. Optionally, the subject does not have a side effect from the method, where the side effect is substantial hair loss, gastrointestinal bleeding, and/or chemo brain.

Another aspect of the invention provides a method of preventing tumor formation in a subject predisposed to a malignancy or having pre-cancer, involving administering an effective amount of SOCS3, a vector that expresses a SOCS3 polypeptide, and/or a SOCS3 inducing agent to the subject, such that tumor formation is prevented.

In one embodiment, the subject predisposed to a malignancy has familial adenomatous polyposis or is a carrier for a BRCA1 or BRCA2 mutation associated with cancer.

Optionally, the SOCS3 inducing agent is a flavanone.

In certain embodiments, the flavanone is Butin, Eriodictyol, Hesperetin, Hesperidin, Homoeriodictyol, Isosakuranetin, Naringenin, Naringin, Pinocembrin, Poncirin, Sakuranetin, Sakuranin or Sterubin, or any combination thereof.

In one embodiment, the SOCS3 inducing agent is an antibody, or an antigen-binding portion thereof.

In certain embodiments, SOCS3 is either a nucleic acid encoding SOCS3 protein, or a functional fragment thereof, or a SOCS3 protein, or a functional fragment thereof.

In one embodiment, the nucleic acid is a viral vector.

In some embodiments, the SOCS3 inducing agent is a modified SOCS3 fusion protein.

In certain embodiments, the SOCS3, vector that expresses a SOCS3 polypeptide, and/or the SOCS3 inducing agent is administered to the subject via systemic administration, oral administration, enteral administration, and/or topical administration.

Another aspect of the invention provides a method of inhibiting or decreasing a tumor in a subject, involving administering an effective amount of an agent that promotes acetylcholine release to a tumor-associated tissue in the subject and/or to a host tissue of the subject, such that growth of the tumor is inhibited or decreased.

A further aspect of the invention provides a method of inhibiting or decreasing solid tumor growth in a subject, involving selecting a subject having a solid growth tumor and systemically administering an effective amount of an agent that promotes acetylcholine release to the subject, such that growth of the solid tumor is inhibited or decreased.

In one embodiment, the tumor is a lung carcinoma, a glioblastoma, a gastric adenocarcinoma, a hepatocellular carcinoma, and/or a melanoma.

In certain embodiments, the invention further involves delivering the agent that promotes acetylcholine release directly to the tumor.

In one embodiment, the subject does not have a side effect from the method, where the side effect is substantial hair loss, gastrointestinal bleeding, and/or chemo brain.

Another aspect of the invention provides a method of preventing tumor formation in a subject predisposed to a malignancy or having pre-cancer, the method involving administering an effective amount of an agent that promotes acetylcholine release to the subject, such that tumor formation is prevented.

In one embodiment, the subject predisposed to a malignancy has familial adenomatous polyposis or is a carrier for a BRCA1 or BRCA2 mutation associated with cancer.

In certain embodiments, the agent that promotes acetylcholine release is AR-R 17779 hydrochloride; 4BP-TQS; A 582941; A 844606; 3-Bromocytisine; DMAB-anabaseine dihydrochloride; GTS 21 dihydrochloride; PHA 543613 hydrochloride; PHA 568487; PNU 282987; S 24795; SEN 12333; TC 1698 dihydrochloride; A 85380 dihydrochloride; 3-Bromocytisine; CC4; 5-Iodo-A-85380 dihydrochloride; (−)-Nicotine ditartrate; 3-pyr-Cytisine; RJR 2403 oxalate; SIB 1508Y maleate; TC 2559 difumarate; Varenicline tartrate; A 844606; A 85380 dihydrochloride; 4-Acetyl-1,1-dimethylpiperazinium iodide; 1-Acetyl-4-methylpiperazine hydrochloride; (+)-Anabasine hydrochloride; (±)-Anatoxin A fumarate; 3-Bromocytisine; Carbamoylcholine chloride; CC4; Cisapride; (−)-Cytisine; DMAB-anabaseine dihydrochloride; (±)-Epibatidine; (−)-Lobeline hydrochloride; RJR 2429 dihydrochloride; Sazetidine A dihydrochloride; SIB 1553A hydrochloride; Tropisetron hydrochloride; UB 165 fumarate; Donepezil hydrochloride; Ambenonium dichloride; Galanthamine hydrobromide; PE 154; Phenserine; Physostigmine hemisulfate; Rivastigmine tartrate; and/or Tacrine hydrochloride, and any combinations thereof.

In some embodiments, the agent that promotes acetylcholine release to the subject is administered to the subject via systemic administration, oral administration, enteral administration, and/or topical administration, optionally where the agent that promotes acetylcholine release is administered to nerve fibers, optionally nerve fibers associated with the tumor.

In one embodiment, the tumor is a solid tumor. Optionally, the solid tumor is a melanoma, a lung cancer, a gastric cancer, a liver cancer, a colon cancer, an esophageal cancer, and/or a pancreatic cancer.

In some embodiments, the tumor-associated tissue is a host tumor bed, a macrophage population, a vascular tissue, a neural tissue, or a muscle tissue.

An additional aspect of the invention provides a method of inhibiting pathological blood vessel growth involving administering to a subject with pathological blood vessel growth an agent that promotes acetylcholine release, thereby inhibiting pathological blood vessel growth.

A further aspect of the invention provides a method of inhibiting pathological blood vessel growth involving administering an agent that promotes acetylcholine release to a subject with pathological blood vessel growth, thereby inhibiting pathological blood vessel growth.

Another aspect of the invention provides a method of treating an autoimmune disease or sepsis involving administering to a subject with an autoimmune disease or sepsis an agent that promotes acetylcholine release, thereby treating the autoimmune disease or sepsis.

In one embodiment, the autoimmune disease is associated with pathological blood vessel growth. Optionally, the agent is administered to a host tissue.

In one embodiment, the autoimmune disease is retinopathy. Optionally, the retinopathy is retinopathy of prematurity, diabetic retinopathy or age related macular degeneration.

In another embodiment, the autoimmune disease is juvenile rheumatoid arthritis (JRA).

In one embodiment, the subject has sepsis.

Another aspect of the invention provides a kit that includes an agent that promotes acetylcholine release, and instructions for its use in inhibiting pathological blood vessel growth and/or pathological neovascularization.

A further aspect of the invention provides a kit that includes an agent that promotes acetylcholine release, and instructions for its use in inhibiting tumor growth.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass and associated neuronal cells/fibers as well as tumor associated macrophages and other cells of the tumor microenvironment. In one embodiment, the tumor is a malignant tumor, i.e., a cancer having the ability to metastasize (i.e. a metastatic tumor). In a separate embodiment, a tumor is benign or nonmalignant (i.e. non-metastatic tumor).

As used herein, the phrase “tumor-associated tissue” includes any tissue in the local vicinity of a tumor within a subject, including, e.g., tumor bed tissue, such as vascular tissue, neural tissue/fiber(s), muscle tissue, tumor associated macrophages, other tumor microenvironment cells and other tissue that supports or is otherwise within the vicinity of a tumor in a subject.

As used herein, the term “host tissue” refers to any tissue of a subject. In one embodiment, the host tissue is stromal tissue of the subject.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

In certain embodiments, “proliferative disease” or “cancer” as used herein is meant, a disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

The term “neoplasia” or “hyperproliferative disorder” includes malignancies characterized by excess cell proliferation or growth, or reduced cell death. In specific embodiments, the term “cancer” includes but is not limited to carcinomas, sarcomas, leukemias, and lymphomas. The term “cancer” also includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor. Tumors include solid tumors (i.e., non-blood tumors) and blood tumors.

The term “pathological blood vessel growth” as used herein, refers to the creation of a new blood vessel either from an existing blood vessel or the creation of a new blood vessel where there is no pre-existing blood vessel. Pathological blood vessel growth refers to blood vessel growth related to a disease, e.g., a subject having a solid tumor. In one embodiment, pathological blood vessel growth refers to neovascularization (new growth not derived from a pre-existing blood vessel). In one embodiment, pathological blood vessel growth refers to the creation of a new blood vessel either from an existing blood vessel.

The sequences of full-length human SOCS3 mRNA and polypeptide sequences are known in the art and available as NCBI Reference Sequences NM_003955.4 and NP_003946.3, respectively.

As used herein, the term “SOCS3 inducing agent” refers to an agent that is capable of increasing SOCS3 mRNA expression or SOCS3 protein levels. Examples of a SOCS3 inducing agent include, but are not limited to, a modified SOCS3 fusion protein, a SOCS3 protein mimetic, or an anti-SOCS3 agonist antibody, or antigen binding fragment thereof. The ability of a SOCS3 inducing agent to increase levels of SOCS3 (protein or nucleic acid) may be tested according to standard methods, including those described in the Examples provided herein.

Cells and/or subjects may be treated and/or contacted with one or more anti-neoplastic treatments including, surgery, chemotherapy, radiotherapy, gene therapy, immune therapy or hormonal therapy, or other therapy recommended or proscribed by self or by a health care provider.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A. et al. (1991) Sequences of Proteins of Immunological Interest, Fifth Edition, U.S. Department of Health and Human Services, NIH Publication No. 91-3242; and Chothia. C. et al (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two (3-sheets formed of about seven β-strands, and a conserved disulphide bond (see, e.g., A. F. Williams and A. N. Barclay (1988) Ann. Rev. Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with TWEAK or a TWEAK receptor.

The VH or VL chain of the antibody can further include all or part of a heavy or light chain constant region, to thereby form a heavy immunoglobulin chain (HC) or light immunoglobulin chain (LC), respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, EC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins, or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

As used herein, the term “immune cells” generally includes white blood cells (leukocytes) which are derived from hematopoietic stem cells (HSC) produced in the bone marrow “Immune cells” includes immune effector cells, e.g., lymphocytes (T cells, B cells, natural killer (NK) cells) and myeloid-derived cells (neutrophil, eosinophil, basophil, monocyte, macrophage, dendritic cells). The term “immune effector cell,” as used herein, refers to a cell that is involved in an immune response, e.g., in the promotion of an immune effector response. Examples of immune effector cells include T cells, e.g., alpha/beta T cells and gamma/delta T (γδ T) cells, B cells, natural killer (NK) cells, natural killer T (NK-T) cells, mast cells, and myeloic-derived phagocytes. “Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCMX} central memory T (T_{CM} effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

The term “human antibody” as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95).

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

The term “subject” includes organisms which are capable of suffering from cancer or other disease of interest who could otherwise benefit from the administration of a compound or composition of the invention, such as human and non-human animals. Preferred human animals include human patients suffering from or prone to suffering from cancer or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. A human subject can be referred to as a patient.

By “population” is meant at least 2 cells. In a preferred embodiment, population is at least 5, 10, 50, 100, 500, 1000, or more cells.

By “obtain” is meant purchasing, synthesizing, or otherwise acquiring.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

By “reference” is meant a standard or control condition.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Boil. Chem. 260:2605-2608 (1985); Rossolini et al., Mol. Cell Probes, 8:91-98 (1994)). The term nucleic acid is used interchangeably with gene, cDNA, mRNA, oligonucleotide, and polynucleotide.

The terms “identical” or percent “identity,” in the context of two or more nucleic acids or polypeptide sequences, refer to two or more sequences or subsequences that are the same or have a specified percentage of amino acid residues or nucleotides that are the same (e.g., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

As used herein, the term “recombinant DNA molecule” as used herein refers to a DNA molecule, which is comprised of segments of DNA joined together by means of molecular biological techniques.

By the term “analog” is meant a molecule that is not identical, but has analogous functional or structural features.

By “small molecule” is meant a compound having a molecular weight of no more than about 1500 daltons, 1000 daltons, 750 daltons, 500 daltons. A small molecule is not a nucleic acid or polypeptide.

By “detecting” or “detection” and the like is meant the process of performing the steps for determine if an analyte is present. The amount of analyte present may be none or below the level of detection of the method.

By “ameliorate” is meant decrease, suppress, attenuate, diminish, arrest, or stabilize the development or progression of a disease.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. More than one dose may be required for prevention of a disease or condition.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition. More than one dose may be required for prevention of a disease or condition.

Various methodologies of the instant invention include step that involves comparing a value, level, feature, characteristic, property, etc. to a “suitable control”, referred to interchangeably herein as an “appropriate control”. A “suitable control” or “appropriate control” is a control or standard familiar to one of ordinary skill in the art useful for comparison purposes. In one embodiment, a “suitable control” or “appropriate control” is a value, level, feature, characteristic, property, etc. determined prior to performing contacting of a cell, tissue, tumor and/or subject with a modified SOCS3 fusion protein or inducer of SOCS3 as described herein. For example, a SOCS3 level, or related transcription rate, mRNA level, translation rate, protein level, biological activity, cellular characteristic or property, genotype, phenotype, etc. can be determined prior to introducing a modified SOCS3 fusion protein or inducer of SOCS3 of the invention into a cell, tissue, tumor or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

The phrase “pharmaceutically acceptable carrier” is art recognized and includes a pharmaceutically acceptable material, composition or vehicle, suitable for administering compounds used in the methods described herein to subjects, e.g., mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Usually, the treatment will involve the administration of a compound preferentially active or safe in patients with certain profiles or responses of the enriched population of tumor-initiating cells from the subject to contact with an agent ex vivo. The administration may involve a combination of compounds. Thus, in preferred embodiments, the method involves identifying such an active compound or combination of compounds, where the compound is less active or is less safe or both when administered to a patient having a different profile.

Also in some embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum.

Also in this context, a “suitable dosage level” refers to a dosage level that provides a therapeutically reasonable balance between pharmacological effectiveness and deleterious effects. Often this dosage level is related to the peak or average serum levels resulting from administration of a drug at the particular dosage level.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and 1B show the murine conditional knockout and re-supplementation with Socs3 strategy employed. FIG. 1A shows the targeting strategy employed for the generation of conditional knockout mice of Socs3 using neuronal-specific nestin Cre (Socs3^{flox/flox}/nestin-Cre), while FIG. 1B shows that neuronal-specific nestin Cre targeted Socs3 deficiency mice (Socs3^{flox/flox}/nestin-Cre) were implanted subcutaneously with mouse tumor carcinoma cells, prior to assessing tumor growth in the murine model system.

FIGS. 2A to 2C show that neuronal deficiency of Socs3 promoted melanoma growth, whereas exogenous Socs3 (lenti-Socs3) inhibited melanoma growth.

FIGS. 3A to 3E show that neuronal deficiency of Socs3 promoted lung carcinoma growth and metastasis formation (in examined lung), whereas exogenous Socs3 (lenti-Socs3) inhibited lung carcinoma growth and metastasis formation.

FIG. 4 shows that lenti-Socs3 treatment inhibited lung carcinoma growth on Socs3f/f and Socs3nes knockout mice. In the chart provided in FIG. 4, the symbols refer to the following: triangles refer to mice with no neural mSOCS3 (Socs3nes knockout (ko) mice); “x” refers to wild type mice with normal mSOCS3 plus extra hSOCS3 injected 4 days prior to tumor inoculation; diamonds refer to wild type mice with normal mSOCS3; and circles refer to hSOCS3 injected locally 4 days prior to tumor implantation.

FIG. 5 shows that exogenous Socs3 inhibited lung carcinoma growth on Socs3 f/f and Socs3 nes ko mice (when tissue around tumor and tumor cells was treated with lenti-Socs3).

FIGS. 6A to 6C show that blood vessel around and inside the tumor on Socs3 f/f and Socs3 nes ko mice were similar. FIG. 6A shows that the blood vessel around similarly sized tumors from wild-type and knockout mice were similar, while FIG. 6B shows that there was no difference in Pecam mRNA levels (a blood vessel marker) inside of tumors of wild-type and knockout mice. FIG. 6C shows that the corresponding density of Pecam. immunohistochemistry staining of tumors from wild-type and knockout mice was also similar.

FIG. 7 shows that pro-angiogenic growth factors and receptors were down-regulated rather than up-regulated in the tumor from Socs3 nes ko. Meanwhile, such levels were generally restored when Socs3 was (re)introduced using lentivirus.

FIG. 8 shows Bodian staining of nerve fiber on tumors from Socs3 f/f and Socs3 nes ko mice, which demonstrated that more nerve fiber was observed in knockout mice than in wild-type mice; however, the nerve fibers of knockout mice appeared thinner, indicating that these unhealthy nerve fibers promoted tumor growth.

FIGS. 9A and 9B show that Botulinum toxin (Botox) injection (0.25 U/20 g mice) 7 days earlier than tumor cell injection promoted tumor seeding, indicating dysfunction of motor neuron promoted tumor seeding.

FIGS. 10A to 10C show that injection of lentiviral Socs3 following Botox injection but before tumor cell injection in wild-type Socs3 mice blocked the effect of Botox injection upon tumor engraftment/growth, indicating that Botox and Socs3 worked through similar mechanism.

FIG. 11 demonstrates the negative impact of nestin-conditional Socs3 knockout upon acetylcholine receptor (AChR) levels, where “purple” indicates mice carrying wild-type Socs3 alleles, “Green” corresponds to mice harboring Socs3 knockouts and “+Socs3” indicates lentiviral overexpression of Socs3.

FIG. 12 diagrams the points of interaction between motor neurons and muscle fibers.

FIG. 13 shows muscle cross-section images obtained for a double-fluorescent Cre reporter combined with nestin-conditional Socs3 knockout. In cells in which Socs3 expression was knocked out, GFP was expressed (Socs3 knockout was observed in the axon on the neuromuscular junction (NMJ)). Such GFP expression also coincided with expression of beta III tubulin.

FIG. 14 demonstrates the negative impact of nestin-conditional Socs3 knockout upon myelin basic protein (MBP) levels, where “purple” indicates mice carrying wild-type Socs3 alleles, “Green” corresponds to mice harboring Socs3 knockouts and “+Socs3” indicates lentiviral overexpression of Socs3.

FIG. 15 demonstrates the negative impact of nestin-conditional Socs3 knockout upon both Plexin 4A and Notch 1 levels, where “purple” indicates mice carrying wild-type Socs3 alleles, “Green” corresponds to mice harboring Socs3 knockouts and “+Socs3” indicates lentiviral overexpression of Socs3.

FIG. 16 depicts a model in which reduced expression of Socs3 in neuronal cells results in increased cytokine (e.g., LI6) expression/release, also including an “X” oncogene that is secreted from neuronal cells or induced by cytokines, which, in turn, lowers VEGFA, VEGFC, TGFb1R and nAChR levels, resulting in enhanced tumor cell engraftment and growth.

FIG. 17 shows correlations of different expression status of VEGF-A and VEGF-C with clinicopathologic parameters for gastric cancer.

FIGS. 18A to 18D show the impact of administration of the flavanone naringenin to tumor-bearing mice. FIG. 18A shows that naringenin administration (which increased Socs3 levels) significantly decreased tumor growth in both Socs3-containing and Socs3 nestin conditional knockout mice, when treatment was administered on day 4. FIG. 18B shows that naringenin administration shrank tumors at the day 12-13 timepoint examined, with 100% of tumors of Socs3f/f mice and 60% of tumors in Socs3 nestin conditional knockout mice observed to have shrunk at day 13 of the timecourse. FIG. 18C shows raw tumor size data for the animals of FIGS. 18A and 18B. FIG. 18D also shows that naringenin administration significantly decreased LLC tumor growth in both Socs3 wild-type and Socs3 nestin conditional knockout mice.

FIG. 19 depicts a diagram of monocyte differentiation leading to either the M1 (anti-tumor, cytotoxicity, immune-stimulating) or M2 (angiogenesis, tumor promotion, suppression of M1 and adaptive immunity) outcome.

FIG. 20 shows a schematic diagram of the process of fluorescence activated cell sorting (FACS), in which green cells are positively charged and therefore sorted towards an anode plate while negatively charged red cells are sorted toward a cationic plate.

FIG. 21 shows a control FACS outcome when sorting was performed upon unstained cells, resulting in essentially all cells being sorted into one, non-fluorescent quadrant. Thus, entirely macrophages (M1 macrophages along the x-axis) were detected along both axes.

FIG. 22 shows that Socs3 neuronal deficient mice had reduced relative M1 levels as compared to total macrophage levels, while such Socs3 neuronal deficient mice also showed increased tumor growth. Thus, it was observed that Socs3 neuronal deficiency reduced M1 macrophages, but also likely promoted M2 macrophages in tumors.

FIG. 23 shows that the Socs3 inducer Naringenin promoted M1 macrophage levels but did not change M2 macrophage levels in wild-type tumor.

FIG. 24 shows that an α7AChR agonist (AR-R17779) also promoted M1 macrophage levels but did not change M2 macrophage levels in wild-type tumor.

FIG. 25 shows that the Socs3 inducer Naringenin promoted M1 macrophage levels while also reducing M2 macrophage levels in Socs3 neuronal deficient tumor.

FIG. 26 shows the α7AChR agonist AR-R17779 promoted M1 macrophage levels while not promoting M2 macrophage levels in Socs3 neuronal deficient (knockout) tumor.

FIG. 27 shows the effect of Socs3 nestin-conditional knockdown and lentiviral overexpression of Socs3 upon levels of the M1 macrophage markers iNOS, IL 6, IL 1b and CXCL10. Notably, both Socs3 knockout and lentiviral overexpression had significant impact upon levels of iNOS, IL6 and IL 1b markers.

FIGS. 28A to 28D show that Socs3 mRNA expression was induced in neuronal layers in the OIR model. FIG. 28A shows a schematic diagram of OIR. Neonatal mice were exposed to 75% oxygen from postnatal day (P) 7-12 to induce vessel loss and returned to room air from P12-P17 to induce maximum pathologic neovascularization at P17. FIG. 28B shows Socs3 mRNA expression in P17 retinal layers from laser-capture-micro-dissection (LCM) from OIR versus age-matched normoxia (Norm) control retinas (n=6 per group). Socs3 mRNA was upregulated in RGC (P=0.008) and INL (P=0.006), but not in ONL in OIR compared with normoxia control. Images on the left show representative retinal cross sections from normoxia and OIR retinas stained with Isolectin IB4 (red) and DAPI (blue), with dotted lines highlighting the areas for LCM. FIG. 28C shows that Socs3 was decreased by 80% by crossing Socs3 flox/flox (Socs3 f/f) mice with nestin-Cre driven mice (Socs3 Nes-ko). Decreased Socs3 levels were confirmed in whole retinas by western blot (p=0.0006, n=3). FIG. 28D shows P17 OIR retinal cross sections from Socs3 Nes-ko;mTmG reporter show that Socs3 was knocked out in all the neuronal layers (labeled with GFP). OIR: oxygen-induced retinopathy; RGC, retinal ganglion cells; IPL, inner plexiform layer; INL, inner nuclear layer; OPL, out plexiform layer; ONL, outer nuclear layer.

FIGS. 29A to 29P show that neuronal/glial Socs3 attenuated pathologic neovascularization in the OIR model. Representative retinal flat-mounts from P17 OIR Socs3f/f (FIGS. 29A to 29D) and Socs3 Nes-ko (FIGS. 29E to 29H) retinas stained with isolection IB4 (red) for endothelial cells. The areas of neovascularization (NV) (FIGS. 29B and 29F) and vaso-obliteration (VO) (FIGS. 29C and 29G) were highlighted for quantification (white) using Image J and Photoshop. FIGS. 29D and 29H show enlarged pathologic neovessels. Quantification of pathologic NV (FIG. 29I) showed Socs3 attenuated pathologic neovascularization (NV) (p=0.002, n=14-27) and dampened central VO area (FIG. 29J) in OIR at P17 (p=0.004, n=10-27). Between Socs3 f/f and Socs3 Nes-ko retinas, the retinal VO areas were comparable at P12 (FIGS. 29K, 29L and 29O) (p=0.16, n=7-10) and there was no significant difference in normal developmental retina areas at P7 (FIGS. 29M, 29N and 29P) (p=0.94, n=5-6). Scale bar: 1000 μm. Norm: normoxia; OIR: oxygen-induced retinopathy; RGC: retinal ganglion cells; INL: inner nuclear layer; ONL: outer nuclear layer.

FIGS. 30A to 30D show that neuronal/glial Socs3 deficiency enhanced Vegfa expression. FIG. 30A shows Vegfa mRNA expression in P17 retinal layers from laser-capture-micro-dissection (LCM) from OIR versus age-matched normoxia (Norm) control retinas. Vegfa mRNA expression was increased in RGC and INL, but not in ONL in P17 retinas layers (LCM) from OIR compared with age-matched normoxic control retinas (n=6 per group). Socs3 suppression in retinal neurons and glia increased Vegfa both at mRNA level (˜3.7 fold, p=0.003, n=3) (FIG. 30B) and protein level (˜3.5 fold, p=0.003, n=3) (FIG. 30C), but not Ngfβ expression (FIG. 30D).

FIGS. 31A and 31B show the impact of knocking out Socs3 in neurons and Müller glial cells and astrocytes. FIG. 31A shows GFAP-labeled activated Müller cells in P17 Socs3 Nes-ko OIR retinas were increased versus Socs3f/f OIR controls. Cross-sections from P17 OIR Socs3 Nes-ko and Socs3f/f controls were stained for endothelial cells with isolectin I1B4 (red), activated Müller cells and astrocytes with anti-GFAP (green) and cell nuclei with DAPI (blue). White stars: OIR proliferative vessels; White arrow: astrocytes or end-feet of activated Müller cells; Open arrowheads: activated Müller cells; Scale bar: 50 μm. FIG. 31B shows GFAP-labeled astrocytes and end-feet of activated Müller cells in flat-mounted P17 Socs3 Nes-ko and Socs3f/f OIR retinas. White arrow: astrocytes; Open arrowheads: end-feet of activated Müller cells; White stars: OIR proliferative vessels; Scale bar: 50 μm.

FIGS. 32A to 32D show that knocking out Socs3 in neurons and glia increased STAT3 and VEGF signaling. In FIG. 32A, the mRNA expression of HIF-1α target genes, Epo and Angptl4, were at comparable levels in P17 Socs3 Nes-ko OIR retinas and littermate Socs3f/f OIR retinas (n=6 per group). FIG. 32B shows that in P17 Socs3 Nes-ko OIR retinas, representative western blots showed that phospho-STAT3 (pSTAT3) was highly increased versus Socs3f/f OIR retinas. Band densities for western blot were quantified using image J in the bottom panel (p=0.01). FIG. 32C shows that in P17 Socs3 Nes-ko OIR retinas, representative western blots showed that downstream of VEGF signaling, phospho-ERK (pERK) was highly increased versus Socs3f/f OIR retinas. Band densities for western blot were quantified using image J in the right panel (p=0.003). FIG. 32D shows a schematic representation of neuronal/glial SOCS3 functioning as a modulator of angiogenic activation via the VEGF signaling pathway. SOCS3 is one of the STAT3 transcriptional targets and can inhibit STAT3 transcription activity through JAK kinase inhibition. Loss of SOCS3 decreased the inhibition of JAK kinase activity. This led to upregulation of phosphorylation of STAT3 by activated JAK kinase, then activated phosphor-STAT3 translocate to nuclear and enhance other downstream target genes, such as VEGF expression. Overexpressed VEGF from neuronal/glial cells likely acted on other cells, such as endothelial cells, to lead to neovascularization. Thus, SOCS3 is identified as likely to be a new critical factor in modulating neurovascular crosstalk in regulating retinopathy, as well as a drug target to inhibit in developing future therapeutics to treat retinopathy.

FIG. 33 shows that alpha-bungarotoxin (a-BTX), an inhibitor of the achetylcholine receptor (AChR) promoted tumor growth in both LLC tumor cells and in B16F10 tumor cells.

FIG. 34 shows that AR-R17779, an alpha-7 nicotinic AChR agonist, significantly prevented tumor growth when administered to LLC tumor cells.

FIG. 35 shows that AR-R17779 treatment also decreased tumor size on day 10 post-implantation.

FIG. 36 shows that alpha-7 nicotinic AChR knockout mice exhibited LLC tumors that grew faster than in wild-type mice.

FIG. 37 shows that donepezil, an inhibitor of ACh esterase also prevented tumor growth in both LLC tumor cells and in B16F10 tumor cells.

FIG. 38 shows the differential effects observed for a-BTX (as an inhibitor of AChR) and donepezil (as drug promoting ACh release) in both LLC tumor cells and B16F10 tumor cells in treated mice, as compared to growth of tumors in control animals.

FIG. 39 shows that genetic knockout approaches to reducing or eliminating alpha-7 AChR expression resulted in apparent dose-dependent increases in rates of tumor growth. Rates of LLC tumor growth were observed to be greatest in alpha-7 AChR knockout mice (where a statistically significant increase was seen), but a trend towards elevated tumor growth was also observed in even mice heterozygote for the alpha-7 AChR knockout (even though not statistically significant in the instant experiment). Thus, alpha-7 AChR was demonstrated as modulating LLC tumor reduction effects.

FIGS. 40A and 40B show the effects of donepezil or lenti-Socs3 treatments on alpha-7 AChR knockout mice. FIG. 40A shows the observed impact of donepezil treatment upon wild-type, heterozygote alpha-7 AChR knockout mice and homozygote alpha-7 AChR knockout mice. Donepezil (a general promoter of ACh release) was observed to have a dramatic impact on modulation of tumor growth rates, especially when alpha-7 AChR was inactivated, thereby confirming that alpha-7 AChR plays a large role (even if likely not the only form of AChR involved) in the ACh pathway-related modulation of tumor growth that was observed. Consistent with the trend for heterozygote mice initially observed above, treatment of even heterozygote alpha-7 AChR knockout mice with donepezil produced a significant reduction in tumor growth in such animals (as compared to PBS-treated controls). Meanwhile, a robust reduction of tumor growth rate was observed for homozygous alpha-7 AChR knockout mice treated with donepezil (as compared to PBS-treated controls). FIG. 40B demonstrates that similar effects were also observed for lentiviral Socs3-treated mice, noting while AChR knockout promoted tumor growth, a return to wild-type levels of tumor growth could be restored to AChR knockout mice when lentiviral Socs3 treatment was performed.

FIG. 41 shows that anti-tumor macrophages (M1 macrophages) increased in suppressed tumors, consistent with the role that ACh is known to play in the inflammatory response, and confirming the involvement of tumor associated macrophages in the newly observed effects described herein.

FIG. 42 shows that tumor-infiltration dendritic cells (TIDC; macrophages) increased in suppressed tumors, consistent with the role of TIDCs in maintaining antitumor immunity. The TIDC population is noted as CD45⁺; F4/80⁺; CD11c⁺ cells, which were contracted in the a-BTX-treated mice and expanded in the donepezil-treated mice, as compared to control mice.

FIG. 43 shows a flow chart demonstrating the pathway by which motor neuron control of tumor growth appears to occur via regulation of tumor-associated macrophages.

FIGS. 44A and 44B are graphs demonstrating that overexpression of Socs3 in myeloid cells suppress both LLC (FIG. 44A) and B16F10 (FIG. 44B) tumor seeding and growth.

FIG. 45 is a series of plots quantifying the tumor volume, total tumor cells isolated from tumors, total CD45 positive cells and percentage of CD45 positive cells vs. total cells in control- or Naringenin-treated tumors. 1,2,3,4,5 represents the individual tumor in each group. In order to determine whether Naringenin influences the infiltrating of different immune cell types into tumors, fluorescence-activated cell sorting (FACS) was conducted on cells isolated from tumors treated with Naringenin or vehicle control. The tumors were significantly suppressed with Naringenin treatment compared with control group based on the tumor volume and total cells isolated from the entire tumors.

FIGS. 46A and 46B are graphs showing that the percentage of natural killer (NK) cells (FIG. 46A) of CD45⁺ cells was higher in the Naringenin-treated group compared as compared to the control-treated group, and there was a very good negative correlation (FIG. 46B) between percent of CD45⁺ NK cells and tumor volume.

FIGS. 47A and 47B are graphs showing that the percentage of CD45⁺ γδ T cells (FIG. 47A) also increased in the Naringenin-treated group and there was a very good negative correlation (FIG. 47B) between percentage of γδ T cells and tumor volume.

FIG. 48 is a graph showing variations in CD8⁺ T cell recruitment into Naringenin-treated tumors.

FIG. 49 is a graph showing that CD4⁺ effector T cells increased in non-responding tumors on immunotherapy. Naringenin treatment did not change the population of CD4⁺ T cells in the tumors.

FIG. 50 is a graph demonstrating that the percentage of B cells of CD45 was very low and the B cell recruitment into the tumor microenvironment was varied between different tumors.

FIG. 51 is a plot showing the results of a drop-seq (single cell analysis) of immune cell infiltration into tumor microenvironments.

### DETAILED DESCRIPTION

The present invention is based, at least in part, upon discovery of a strategy to modulate a host proliferative response by supplementing SOCS3, which has been identified as a native inhibitor of many proliferative pathways. Rather than selectively targeting unique properties of specific tumors, the compositions and methods of the invention target host tumor beds (including vessels and neurons), which are implicated in all tumors.

In general, the present invention provides compositions and methods for inhibiting tumor growth by up-regulating and/or supplementing SOCS3 in tumor and/or tumor-associated tissues of a subject or in cells in vitro. Compounds capable of up-regulating SOCS3, including flavanones, are identified herein, as are methods for identifying additional agents as inducers of SOCS3.

In certain embodiments, the invention describes approaches for design of a drug that serves as an adjuvant to most chemotherapy protocols.

Because a large class of drugs that can have cell specific effects following modification to specify delivery of, e.g., the antibody of contemplated antibody-SOCS3 fusion proteins, is contemplated, applications also include diabetes, retinopathy and AMD. Broader applications for SOCS3 are also contemplated, as SOCS3 also regulates downstream effects of inflammation, which modifies a vast number of pathological processes. In fact, SOCS3 was reported to have potent anti-inflammatory properties in a model of sepsis. Because it is ubiquitously expressed under stress, it likely partakes in disease processes such as Crohn's colitis, psoriasis, asthma and other auto-immune conditions. Furthermore, ex vivo evidence supports a regulatory role of SOCS3 on insulin receptor signaling with potential implications for diabetes, another disease associated with inflammation.

The compositions and methods of the current invention are applicable to many if not most solid tumors. There are few drugs at present that target the host response to cancer.

Treatment of JRA with the compositions and methods of the instant invention is also contemplated. The current standard-of-care treatment for JRA is MTX and anti-TNF drugs, which are not completely effective and have considerable unintended effects. The compositions and methods of the invention provide anti-inflammatory drugs in a new class for JRA treatment.

In addition, treatment of AMD and retinopathies with the compositions and methods of the instant invention is also contemplated. There is no good drug for diabetic retinopathy, while AMD can be treated with anti-VEGF therapy using a monthly injection into the vitreous at the end stage. SOCS3-related compositions and methods of the instant invention are therefore contemplated to improve upon the standard-of-care.

Treatment of sepsis with the compositions and methods of the instant invention is also contemplated. There are no reliable treatments for sepsis. An earlier publication suggests that SOCS3 treatment effectively prevent death from sepsis. Thus, SOCS3-related compositions and methods of the instant invention are also contemplated to improve upon the standard-of-care for sepsis.

There is a need for anti-angiogenic drugs that can inhibit pathological neovascularization as occurs in tumors and in retinopathy without inhibiting normal angiogenesis. It is also important to develop such anti-angiogenic inhibitors that do not block a single pathway (such as anti-VEGF), as they have been proven to be minimally effective long term for halting or controlling tumor growth. As described herein, it has been found that inhibition of SOCS3 in the host vessels of transgenic mice dramatically increased tumor growth and pathological angiogenesis in retinopathy. More importantly, overexpression of SOCS3 (using lentivirus) in tumor beds of Tie2-SOCS3ko and control mice suppressed tumor growth 4.5-fold as compared to control mice (non-coding lentivirus), and in 20% of mice completely prevented tumor growth. Independent of neovessels, tumor cells intrinsically suppressed SOCS3 to evade its negative regulatory control. Therefore, delivery of SOCS3 both to pathological neovessels, the tumor itself and to host tissues potentiates the beneficial effect observed. A predominant role for SOCS3 in regulating tumor growth, and other vascular proliferative disorders, has thus been identified. Modified SOCS3 proteins that are active intracellularly to inhibit tumor and pathological blood vessel growth are also contemplated and described herein.

### Retinal Neuronal SOCS3 Governs VEGF Signaling and Neovascular Growth

Accumulating evidence indicates that retinal neuroglia and neural cells contribute to neovascularization in proliferative retinopathy, but the controlling molecular interactions have, to date, not well known. In certain aspects of the instant invention, a novel mechanism by which neurons influence neovascularization through neuronal and glial suppressor of cytokine signaling 3 (SOCS3) has been identified. As disclosed herein, it was discovered that Socs3 expression was upregulated in the retinal ganglion cell and inner nuclear layers in oxygen-induced retinopathy. Specifically, neuronal/glial Socs3-deficient mice with oxygen-induced retinopathy exhibited significantly increased pathologic retinal neovascularization and reduced vaso-obliterated retinal areas, which indicated that loss of neuronal/glial SOCS3 increased both retinal vascular re-growth and pathological neovascularization. In neuronal/glial Socs3 deficient mice with oxygen-induced retinopathy, retinal vascular endothelial growth factor A (Vegfa) expression was increased versus Socs3 flox/flox controls, indicating that Socs3 suppressed Vegfa during pathologic conditions in neuronal and glial cells, including Müller glial cells and astrocytes. Lack of neuronal/glial SOCS3 resulted in high levels of activated phospho-STAT3, which led to increased expression of its transcription target Vegfa, and elevated phospho-ERK levels, which indicated that enhanced VEGF signaling occurred in retinas lacking neuronal/glial SOCS3. Thus, as also detailed elsewhere herein, neuronal/glial SOCS3 suppressed endothelial cell activation through suppression of STAT3 mediated neuronal/glia VEGF secretion, resulting in less vascular endothelial cell VEGF-induced ERK activation and angiogenesis. These results remarkably identified neuronal/glial SOCS3 as a regulator of neurovascular interaction and pathologic retinal angiogenesis, apparently via titration of VEGF signaling.

It is specifically noted that neurovascular interactions have been described as important in the maintenance of the nervous system, and defects in this relationship can lead to disease, such as stroke (1), Alzheimer's disease (2), and epilepsy (3). In the retina, which is part of the central nervous system, accumulating evidence has indicated that dysregulated cross-talk between the vasculature and retinal neuroglia, photoreceptors, and other neural cells in diabetes might contribute to the pathogenesis of diabetic retinopathy (4-6). Similarly, in preterm neonates, immature retinas have been identified as susceptible to insults that disrupted both neural and vascular growth, leading to proliferative retinopathy of prematurity (7). While many factors have been suggested as mediating neurovascular crosstalk, including growth factors, hydrogen, potassium, neurotransmitters, adenosine, arachidonic acid metabolites, nitric oxide, neurotrophins and glutamate (4), neuronal/glial suppressor of cytokine signaling 3 (SOCS3), a negative feedback regulator of inflammation and growth factor signaling (8), was specifically assessed as a neural regulator of pathological retinal vessel growth.

SOCS3 inhibits the cytoplasmic effectors Janus kinase/signal transducers and activators of transcription (JAK/STAT) kinase and deactivates tyrosine kinase receptor signaling (9). It regulates endothelial cell apoptosis (10). Neuronal SOCS3 deletion promotes optic nerve regeneration in adult mice (11). It was previously shown that vascular SOCS3 (Tie2-Cre driven) was an inhibitor of pathologic angiogenesis (12). However, the role of neuronal SOCS3 in controlling neurovascular coupling-mediated pathologic retinal angiogenesis in vivo has heretofore remained unknown.

It was also noted that systemic deletion of Socs3 was embryonically lethal (13). Thus, a conditional knockout of Socs3 was generated in retinal neurons and glia using a Cre/loxP site-specific DNA recombination fate mapping strategy. Socs3 was deleted in neuronal/glial cells by crossing mice expressing the Cre recombinase transgene under the control of the nestin (Nes) promoter with mice carrying Socs3/loxP (Socs3 Nes-ko). Pathologic retinal angiogenesis in these mice was studied using a mouse model of oxygen-induced retinopathy (OIR) (14). Conditional Socs3 Nes-ko mice subjected to OIR had significantly increased levels of pathologic retinal neovascularization versus littermate flox/flox controls (Socs3f/f) but normal retinal vascular development was unaffected. These results indicated that neuronal/glial SOCS3 suppressed retinal angiogenesis in pathologic conditions (with stress) but was dispensable in physiologic vascular development. Lack of SOCS3 in retinal neuronal and glial cells increased vascular endothelial cell STAT3 activation and promoted vascular endothelial growth factor (VEGF) signaling in endothelial cells, leading to proliferative retinopathy. Therefore, neuronal/glial SOCS3 was identified herein as an important factor acting to control release of growth factors, which mediated vascular growth specifically in pathologic contexts.

The interactions among multiple cell types in the retina are known to influence tissue homeostasis and angiogenesis. Impairment of these interactions can contribute to the progression of retinal diseases (5). In the studies described herein, a novel mechanism involved in neurovascular crosstalk was identified, which governed the progression of retinopathy. Specifically, neuronal/glial SOCS3 deficiency promoted pathologic retinal angiogenesis in retinopathy.

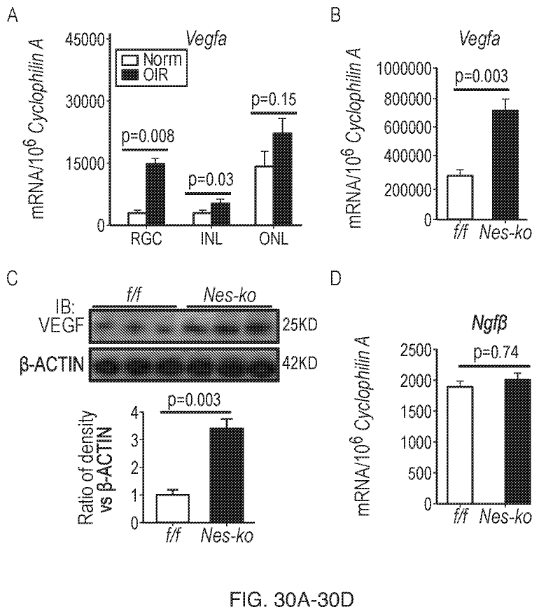

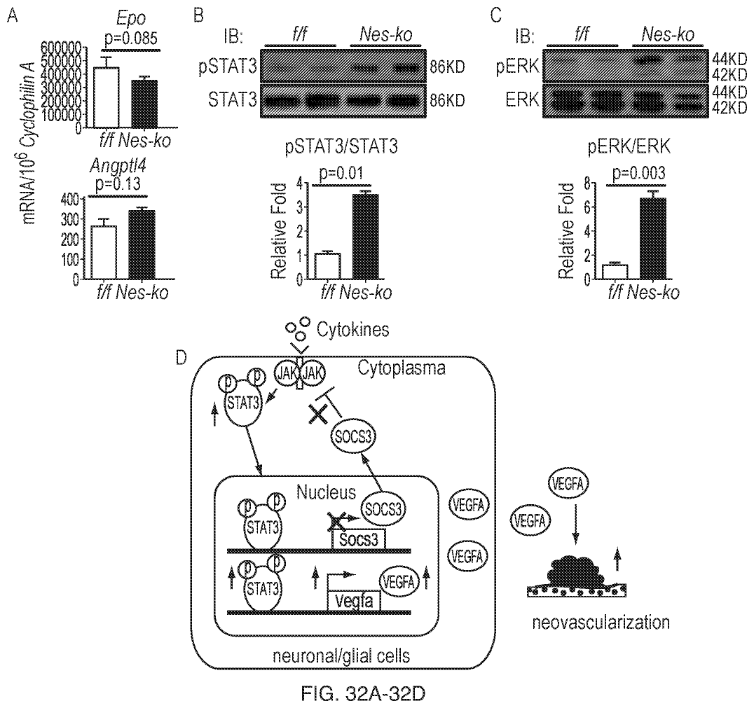

First, it was observed that expression of Socs3, a newly identified endogenous inhibitor of pathologic angiogenesis, was significantly increased in retinal ganglion cells and inner nuclear layers in OIR retinas. Second, it was identified that neuronal/glial Socs3 deficient mice subjected to OIR had increased pathologic retinal neovascularization. These results indicated that neuronal/glial SOCS3 controlled the vascular response in retinopathy. FIG. 32D shows a proposed molecular mechanism in which neuronal/glial SOCS3 mediates retinal neovascularization by modulating the signaling to control neuronal VEGF production. Lack of neuronal/glial Socs3 resulted in loss of JAK kinase inhibition, resulting in elevated phospho-STAT3 levels (FIG. 32B), leading to Vegfa overexpression (FIGS. 30B, 30C). Consequently, phospho-ERK was activated (FIG. 32C), which indicated that neuronal/glial Socs3 suppressed pathologic endothelial proliferation by controlling VEGF secretion through ERK activation mediated by STAT3 activation.

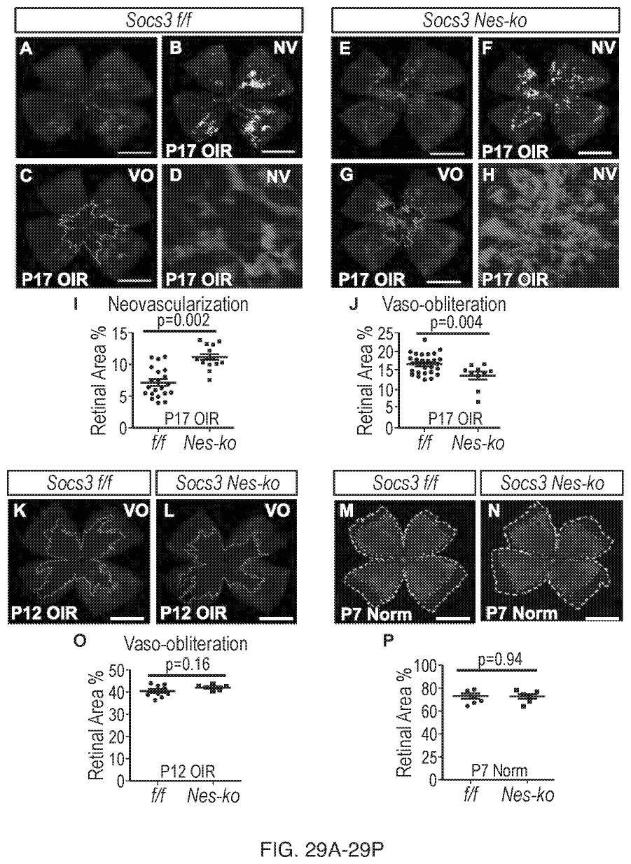

The oxygen-induced retinopathy mouse model has been widely used as an angiogenesis model (26). In this model, during the hypoxic and proliferative phase, VEGF was upregulated mostly in Müller cells of the inner retina (27) and contributed to pathologic neovascularization (18-20). The data obtained herein show that the vaso-obliterated retinal area at P17 was reduced in Socs3 Nes-ko versus Socs3f/f mice at P17, which indicated that the increased vascular repair and re-growth of normal vessels in Socs3 Nes-ko retinas was likely controlled through upregulation of VEGF with loss of neuronal/glial SOCS3. Yet physiological development of retinal vessels was not affected in Socs3 Nes-ko retinas (FIGS. 29M, N and P). These results indicate that neuronal/glial SOCS3 played a regulatory role in reducing angiogenesis and re-growth of normal vessels under pathologic conditions, while not affecting physiologic vascular development, which indicated a novel role of SOCS3 in controlling neurovascular communication in stressed retinas.

VEGF is a soluble factor that interacts with both neuroglia and the vasculature. It affects vascular development, permeability and neovascularization (28). It was previously demonstrated that VEGF expression in the retina played a central role in the development of retinal ischemia-induced ocular neovascularization (19). In situ hybridization localized Vegf mRNA to cell bodies in the inner nuclear layer of the retina, identified morphologically as Müller cells. But the regulation of VEGF in Müller cells has not previously been well studied. Hypoxia-inducible factor 1-alpha (HIF-1α), controlled by hypoxia, has been known to regulate VEGF expression (29). HIF-1α is degraded by the proteasome pathway in normoxia, but has been identified as stabilized under hypoxia, allowing it to translocate from the cytosol to the nucleus (30). To explore if HIF-1α was involved in the regulation of VEGF expression in Socs3 Nes-ko OIR retinas, the expression of other HIF-1α target genes that are involved in OIR was assessed, such as Epo (31, 32) and Anptl4 (33, 34). It was identified that expression levels of the two genes were comparable in both Socs3 Nes-ko OIR and Socs3f/f OIR retinas (FIG. 32A), which indicated that control of VEGF in Socs3 Nes-ko OIR retinas was likely not HIF-1α-dependent. As presented herein, it was identified that the pSTAT3 level was increased with elevated VEGF levels in the neuronal/glial Socs3 deficient retinas (FIGS. 32A to 32D), which indicated that neuroglial cell-secreted VEGF could be regulated by the SOCS3-JAK-STAT3 pathway. A previous study had reported that STAT3 and HIF-1α cooperatively activated HIF-1α target genes including VEGF in tumor cells (35). It was identified herein that VEGFA appeared to be activated primarily by STAT3 and perhaps to a much lesser extent by HIF-1α, although no difference was detected at the level of other HIF-1α target genes, Epo and Angptl4 between Socs3 Nes-ko OIR and Socs3f/f OIR retinas.

In mammalian retinas, neural and vascular tissues are intertwined in functional neurovascular units. Identifying the molecular mechanisms underlying neurovascular cross talk is an important step in understanding pathologic proliferative retinopathy. Neuronal/glial SOCS3 is an important negative regulator of pathologic angiogenesis acting to suppress neuronal/glial VEGF production to regulate vascular endothelial cell activation in a pathological context. Certain aspects of the current observations have demonstrated a novel and specific role for neuronal/glial SOCS3 in controlling neurovascular interactions to limit pathologic angiogenesis in proliferative retinopathy. As such, agents that induce increases in SOCS3 have herein been identified as attractive lead molecules for further development, to inhibit pathologic angiogenesis, and the instant findings also have identified the likely value of screening for additional compounds that induce increases in SOCS3.

### Flavones and Flavanones

Flavones (flavus=yellow), are a class of flavonoids based on the backbone of 2-phenylchromen-4-one (2-phenyl-1-benzopyran-4-one):

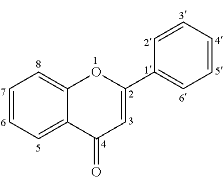 [SMILES: O=c1cc(-c2ccccc2)oc2ccccc12]

Natural flavones include Apigenin (4′,5,7-trihydroxyflavone), Luteolin (3′,4′,5,7-tetrahydroxyflavone) and Tangeritin (4′,5,6,7,8-pentamethoxyflavone), chrysin(5,7-OH), 6-hydroxyflavone, baicalein (5,6,7-trihydroxyflavone), scutellarein (5,6,7,4′-tetrahydroxyflavone), wogonin (5,7-OH, 8-OCH3). Synthetic flavones are Diosmin and Flavoxate.
The flavanones are a type of flavonoids, with the following being the flavanone skeleton:

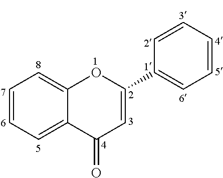

They are generally glycosylated by a disaccharide at position seven to give flavanone glycosides.

Exemplary flavanones include Butin, Eriodictyol, Hesperetin, Hesperidin, Homoeriodictyol, Isosakuranetin, Naringenin, Naringin, Pinocembrin, Poncirin, Sakuranetin, Sakuranin and Sterubin. By way of example as a flavanone, Naringenin has a bioactive effect on human health as antioxidant, free radical scavenger, anti-inflammatory, carbohydrate metabolism promoter, and immune system modulator. It is the predominant flavanone in grapefruit, and has the following structure:

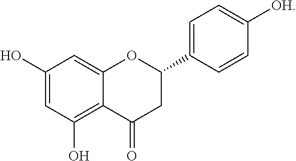 [SMILES: O=C1C[C@@H](c2ccc(O)cc2)Oc2cc(O)cc(O)c21]

It is known and contemplated herein that flavanones may also be derivatized in a number of ways. Exemplary derivatization of Naringenin is disclosed in Yoon et al. (Bioorg Med Chem Lett. 23(1): 232-8). Similarly, derivatives of various other flavenones are also known in the art and presently contemplated.

### Modified SOCS3 Fusion Proteins

Modified SOCS3 fusion proteins can be developed that target the intracellular compartment and ensure continuous delivery to targeted cell populations. These modified proteins can be engineered to evade immune response and enter the intracellular compartment. Homing ligands can optionally be added, targeting preferentially the internalization of SOCS3 fusion proteins to neovessels and tumor cells.

The role of SOCS3 overexpression in models of retinopathy of prematurity and diabetic retinopathy identified herein forms the basis for synthesis and use of SOCS3 fusion proteins. SOCS3 acts in proliferative retinopathies. SOCS3 in host tissue is a generalizable target of pathologic angiogenesis, not limited to cancer. Dose-response experiments can be performed to establish maximal therapeutic effects and an ideal viral delivery method, as well as to assess side effects of SOCS3 over-expression. Single domain targeted antibodies fused with SOCS3 as a delivery system can be engineered. Such engineering can be performed using a technical platform that produces highly specific antibody fragments. Single chain (scFv) and single-domain antibodies (sdAb) can be engineered to maximize specific binding to targeted tissue, achieve rapid clearance and ideal pharmacokinetics. sdAbs derived from the variable regions of camelid heavy-chain antibodies have low molecular weight (12-15 kDa), low nanomolar affinities (Arbabi Ghahroudi et al., 1997) and high temperature and protease stability. Using these advantageous characteristics, SOCS3 as a small protein payload can be fused to an antibody fragment displaying polyvalency and bi-specificity (e.g., Conrath et al., 2001; Zhang et al., 2004). Alone, monovalent (native) sdAbs are rapidly cleared from the circulation by the kidney. It is therefore generally desirable to increase their size (above 65 kDa) to avoid kidney filtration for in vivo targeting. Multiple engineering strategies are available, such as multimerization, fusion to other cargo proteins like SOCS3 or the creation of bi-specific sdAbs, where one of the fragments binds a plasma ‘carrier’ such as albumin in addition to a targeted receptor for specific delivery (Roovers et al., 2007). At least the following two delivery strategies are explicitly contemplated. (1) SOCS3 fusions with VHHs (single-domain antibodies) that internalize into cells via receptor-mediated mechanisms, and (2) SOCS3 fusions with cell-penetrating peptides (e.g., TAT-like peptides; optionally, where such constructs include one or more targeting moieties, e.g., a VHH, aptamer, or other targeting moiety).

SOCS3 fused with cell-internalizing single-domain antibody can be expressed, with two to three cell-internalizing VHHs selected for such fusions (selected from among endothelial cell-internalizing (the majority described) and optionally both EC- and cancer-cell internalizing). Most such VHHs internalize through receptor-mediated mechanism. Internalization can also occur via phospholipid-translocation mechanisms. Alternatively, de-novo panning/selection for specific cell type internalizing sdAbs can be performed. A targeting VHH-SOCS3 fusion that shows the best efficacy in vitro can thereby be selected.

In vivo studies can also be performed, involving design of long plasma half-life SOCS3-VHH fusions (i.e., VHH-Fc-SOCS3 fusion). Alternatively, the original SOCS3-VHH molecule is PEGylated to achieve extended circulating half-life. SOCS3 delivery to specific tissues is generalizable, meaning that by changing the antibody fragment, different tissues can be targeted. While myriad tissues in different disease states could benefit by the compounds and methods of the invention including at least neoplasia and retinopathy diseases and disorders, as well as, e.g., a tissue comprising a neoplasia such as a lung carcinoma, a glioblastoma, a gastric adenocarcinoma, a hepatocellcular carcinoma, or a melanoma, such forms of tissue including, e.g., stromal or other tissues, the importance of SOCS3 expression in host vessels and neurons to control tumor growth is a focus of the instant invention. Targeting antibody fragments against affected host tissues, (as well as tumors (if they have the same antigen as tumors) benefit directly from elevation of SOCS3), provides a powerful adjuvant approach to current chemotherapy.

Markers of pathologic vessels and neurons contemplated for selective VHH include epidermal growth factor receptors (EGFRs) as a target receptor expressed in host vessels, neurons and tumors that has been thoroughly characterized. EGFRs play important roles in tumourigenesis including cell survival, proliferation and angiogenesis (Nicholas et al., 2006). Mutations of the EGFR gene are often associated with EGFR gene amplification. This leads to the expression of a class III mutant EGFR (EGFRvIII), characterized by constitutive autophosphorylation of the tyrosine kinase domain resulting in a ligand-independent receptor signaling (Wikstrand et al., 1998). The appearance of EGFRvIII is associated with poor tumor prognosis (Shinojima et al., 2003). EGFR and EGFRvIII have been exploited as targets for molecular imaging and therapeutic applications in a variety of human cancers (Laskin and Sandler, 2004). In recent years, several IgG antibodies against EGFR, including cetuximab, have proved successful at therapeutic targeting of the EGFR in clinical trials for peripheral tumors.

sdAb cross-reactive against EGFR and EGFRvIII (named EG2) can be fused to SOCS3 and engineered to increase circulation half-life using at least one of the following strategies: (a & b) fusion of one or two EG2 molecules to the human Fc fragment, which is then fused to SOCS3 on the C-terminus, resulting in a mono- or bivalent construct, EG2-hFc-SOCS3, c) or a fusion of human Fc fragment with SOCS3 (C-terminus) without EG2. These constructs can be analyzed in vitro for their kinetic binding properties to EGFR and EGFRvIII and their ability to promote SOCS3 internalization in endothelial and tumor cells. Constructs can be screened ex vivo for optimal efficiency/efficacy using proliferation assays of endothelial cells, aortic sprouting assays and tumors.

In certain examples, SOCS3 can be expressed in fusion with cell-penetrating peptides (e.g., TAT-like peptides, penetratin and/or cell-penetrating peptides described in, e.g., de Figueiredo et al. (IUBMB Life. 2014 Mar. 23. doi: 10.1002/iub.1257), Copolovici et al. (ACS Nano. 2014 Mar. 25; 8(3):1972-94) or elsewhere in the art). Select peptide(s) that mediate direct penetration are fused to SOCS3. Inherent leakiness of neovessels in tumor bed allow accumulation of fusion protein at tumor sites and internalization in host tissues and tumors.

SOCS3 in fusion with a cell-internalizing single-domain antibody can be expressed, with two to three cell-internalizing VHHs initially selected for such fusions (selected from among endothelial cell-internalizing (the majority described) and optionally both EC- and cancer-cell internalizing). Most such VHHs internalize through receptor-mediated mechanism. Internalization can also occur via phospholipid-translocation mechanisms. Alternatively, de-novo panning/selection for specific cell type internalizing sdAbs can be performed.

In vivo targeting of tumor beds and host tissues can also be performed based upon the infra observations of robust inhibition of tumor growth when SOCS3 was injected into the vicinity of tumors (e.g., to tumor bed tissues). Selected constructs can be tested in vivo. Fluorescent labeled EG2-hFc was previously shown to target glioblastomas (Iqbal et al. 2010). Using already available VHHs, their ability to target lung carcinoma, melanomas and pathological retinal neovessels may be confirmed and the ability of SOCS3 biologics to target host tissues in tumor beds can be assessed/achieved while measuring SOCS3 and EG2 concentration at tumor sites. Pharmacokinetic properties and efficacy of selected SOCS3 biologics can also be obtained.

The targeting potential of antibody fragments merged with the potent negative regulatory control of SOCS3, a natural inhibitor of many converging proliferative pathways in cancer, as well as other proliferative and inflammatory conditions is newly recognized and harnessed herein. This is a generalizable approach to controlling inflammatory regulation of many diseases such as autoimmune diseases (JRA etc., cancer, sepsis, AMD, diabetes and other retinopathies).

In certain embodiments, a method of treating cancer comprises administration of a flavonoid, e.g. Naringenin. The administration of the naringenin can be in combination with one or more chemotherapeutic agents, Socs3, immune checkpoint inhibitors and the like.

In certain embodiments, administration of a flavonoid, e.g. Naringenin, increase immune cell infiltration into a tumor. Examples of immune cells include NK cells, T cells, antigen presenting cells.

The immune system can be activated by tumor antigens and, once primed, can elicit an antitumor response which in some cases can result in tumor destruction. Unfortunately, the successful development of antitumor immunity is often hampered by a plethora of factors that can directly determine the adequacy of the immune response. The singular event illustrated by a cytotoxic lymphocyte interacting with a tumor cell holds a background of a series of complex mechanisms, encompassed under the concepts of “immunosurveillance” and “immunoediting”. Critical aspects in the tumor-immune system interface include the processing and presentation of released antigens by antigen-presenting cells (APCs), interaction with T lymphocytes, subsequent immune/T-cell activation, trafficking of antigen-specific effector cells, and, ultimately, the engagement of the target tumor cell by the activated effector T cell. Nevertheless, although often successful in preventing tumor outgrowth, this “cancer-immunity cycle” can be disrupted by artifices involved in immune escape and development of tolerance, culminating with the evasion and proliferation of malignant cells.

T-cell activation relies on the interaction of the T-cell receptor with antigens presented as peptides through the major histocompatibility complex (MHC) by the APC. Tumor antigens are classified as tumor-specific antigens (TSAs), derived from cancer-germline genes, point mutations or oncogenic viruses and unique to tumor cells, or tumor-associated antigens (TAAs), which include differentiation antigens (tyrosinase, gp100, Melan-A/MART-1, carcinoembryonic antigen, prostate-specific antigen, prostatic acidic phosphatase, etc.) and peptides associated with genes overexpressed in tumors (survivin, erbB-2 or CD340, RAGE-1, PRAME, and WTi). HLA downregulation has been shown to result in decreased antigenicity and therefore acts as a mechanism of immune evasion.

While the recognition of peptide-MHC by the TCR plays a central role in the process of T-cell-mediated immunity, additional cell-surface co-receptors are mandatory for the modulation of the immune response, either positively or negatively. Two of these inhibitory co-receptors, called immune checkpoints, are involved in adaptive immune resistance and T-cell tolerance and have been exploited clinically with the development of checkpoint-blocking monoclonal antibodies. The two receptors include the cytotoxic T-lymphocyte-associated protein 4 (CTLA-4, also known as CD152) and the programmed cell death receptor 1 (PD-1 or CD279) and its ligand (PD-L1, also named CD274 or B7-H1)¹⁶. Additional inhibitory receptors include B- and T-cell attenuator (BTLA or CD272), lymphocyte-activation protein 3 (LAG-3 or CD223), T-cell immunoglobulin and mucin protein-3 (TIM-3, also termed hepatitis A virus cellular receptor 2—HAVCR2—or CD366), and V-domain immunoglobulin-containing suppressor of T-cell activation (VISTA, B7H5, or programmed death 1 homolog—PD-1H). Also potential targets for therapeutic manipulation, co-stimulatory receptors associated with positive modulation of the immune synapse include CD27, CD28, CD137, inducible T-cell costimulator (ICOS or CD278), herpesvirus entry mediator (HVEM, also known as tumor necrosis factor receptor superfamily member 14—TNFRSF14), and glucocorticoid-induced TNFR-related protein (GITR or tumor necrosis factor receptor superfamily member 18—TNFRSF18). It is important to highlight, however, that the list of co-receptors and ligands encompasses both co-inhibitory and co-stimulatory molecules other than those aforementioned, some of which are not fully characterized.

The mobilization of these components of the adaptive immune system involved in antitumor immunity, including CD4⁺ helper T cells and CD8⁺ effector T cells, are largely influenced by a milieu of variables that involve intrinsic tumor characteristics, microenvironment factors, and genetic/epigenetic determinants.

NK cells are one of the components of the early, innate immune system NK cells are involved in both the resistance to and control of cancer spread. Since the advent of the cancer immune surveillance concept, the adoptive transfer of immune cells, particularly T cells and natural killer (NK) cells, has emerged as a targeted method of harnessing the immune system against cancer (Kroemer, G., Senovilla, L., Galluzzi, L., Andre, F. & Zitvogel, L. Natural and therapy-induced immunosurveillance in breast cancer. Nat Med 21, 1128-1138, (2015)). NK cells have garnered immense attention as a promising immunotherapeutic agent for treating cancers. NK cells are critical to the body's first line of defense against cancer due to their natural cytotoxicity against malignant cells (Srivastava, S., et al., Cytotherapy 10, 775-783; 2008).

In addition to their cytotoxic capabilities, NK cells serve as regulators of the immune response by releasing a variety of cytokines. In addition, the generation of complex immune responses is facilitated by the direct interaction of NK cells with other cells via various surface molecules expressed on the NK cells.

Similarly to cytotoxic T lymphocytes (CTL), NK cells exert a cytotoxic effect by lysing a variety of cell types (Srivastava, S., Lundqvist, A. & Childs, R. W. Natural killer cell immunotherapy for cancer: a new hope. Cytotherapy 10, 775-783; 2008). These include normal stem cells, infected cells, and transformed cells. The lysis of cells occurs through the action of cytoplasmic granules containing proteases, nucleases, and perforin. Cells that lack MHC class I are also susceptible to NK cell-mediated lysis (H. Reyburn et al., Immunol. Rev. 155:119-125, 1997). In addition, NK cells exert cytotoxicity in a non-MHC restricted fashion (E. Ciccione et al., J. Exp. Med. 172:47, 1990; A. Moretta et al., J. Exp. Med. 172:1589, 1990; and E. Ciccione et al., J. Exp. Med. 175:709). NK cells can also lyse cells by antibody-dependent cellular cytotoxicity.

As noted above, NK cells mediate some of their functions through the secretion of cytokines, such as interferon γ (IFN-γ), granulocyte-macrophage colony-stimulating factors (GM-CSFs), tumor necrosis factor α (TNF-α), macrophage colony-stimulating factor (M-CSF), interleukin-3 (IL-3), and IL-8. NK cell cytotoxic activity is regulated through a balance of activating and inhibitory receptors that enables fine-tuned control of cytotoxic activity, preventing cytotoxicity against healthy cells, while maintaining effective cytotoxic capacity against tumor cells. Indeed, multiple studies have demonstrated the safety of adoptive NK cell transfer and clinical anti-cancer effects, highlighting the potential for NK cells as an effective cancer immunotherapy ((Parkhurst, M. R., et al. Clin Cancer Res 17, 6287-6297 (2011); Ruggeri, L. et al. Science 295, 2097-2100, (2002); Miller, J. S. et al. Blood 105, 3051-3057, (2005; Bachanova, V. et al. Blood 123, 3855-3863, (2014); Rubnitz, J. E. et al. J Clin Oncol 28, 955-959, (2010)). For example, cytokines including IL-2, IL-12, TNF-α, and IL-1 can induce NK cells to produce cytokines. IFN-α and IL-2 are strong inducers of NK cell cytotoxic activity (G. Trinichieri et al., Journal of Experimental Medicine 160:1147-1169, 1984; G. Trinichieri and D. Santoli, Journal of Experimental Medicine 147: 1314-1333, 1977). The presence of IL-2 both stimulates and expands NK cells (K. Oshimi, International Journal of Hematology 63:279-290, 1996). IL-12 has been shown to induce cytokine production from T and NK cells, and augment NK cell-mediated cytotoxicity (M. Kobayashi et al., Journal of Experimental Medicine 170:827-846, 1989).

### Peptidomimetics

In certain embodiments, a method of treating cancer comprises administering a therapeutically effective amount of a Socs3 peptidomimetic. Examples of Socs3 peptidomimetics include LKTFSSKSEYQL (SEQ ID NO: 1) and EYQLVVNAVRKLQESG (SEQ ID NO: 2) ((S. La Manna et al., International Journal of Cancer 143(9) 10.1002/ijc.31594 (May 2018).

In another embodiment, a method of treating cancer comprises administering an effective amount of a modified suppressor of cytokine signaling (SOCS3) fusion protein, a vector expressing a SOCS3 polypeptide, a Socs3 polypeptide or active fragments thereof, a peptidomimetic or combinations thereof, and an immune checkpoint blockade immunotherapeutic targeting, for example, PD-1 (programmed cell death protein 1) e.g. anti-PD-1. The administration of the Socs3 molecules can be in combination with the checkpoint blockade immunotherapeutic or at alternative times and routes. In certain embodiments, the checkpoint blockade immunotherapeutic is an inhibitor of programmed death-ligand 1 (PD-L1), programmed cell death protein 1 (PD-1) and/or CTLA4. Examples of checkpoint inhibitors include: Pembrolizumab, Nivolumab, Atezolizumab, Avelumab. In certain embodiments, one or more checkpoint blockade immunotherapeutics are administered as co-therapeutic agents with other immunotherapy drugs blocking LAG3, B7-H3, KIR, OX40, PARP, CD27, and ICOS. I

Checkpoint blockade immunotherapeutic include antibodies specific for an immune checkpoint or signaling molecule or its ligand and acts as an inhibitor of immune checkpoint suppressive activity or as an agonist of immune stimulatory activity. Such immune checkpoint and signaling molecules and ligands include PD-1, PD-L1, PD-L2, CTLA-4, CD28, CD80, CD86, B7-H3, B7-H4, B7-H5, ICOS-L, ICOS, BTLA, CD137L, CD137, HVEM, KIR, 4-1BB, OX40L, CD70, CD27, CD47, CIS, OX40, GITR, IDO, TIM3, GAL9, VISTA, CD155, TIGIT, LIGHT, LAIR-1, Siglecs and A2aR (Pardoll D M. 2012. Nature Rev Cancer 12:252-264, Thaventhiran T, et al. 2012. J Clin Cell Immunol S12:004). Additionally, antibody binding domains may include ipilimumab and/or tremelimumab (anti-CTLA4), nivolumab, pembrolizumab, pidilizumab, TSR-042, ANB011, AMP-514 and AMP-224 (a ligand-Fc fusion) (anti-PD1), atezolizumab (MPDL3280A), avelumab (MSB0010718C), durvalumab (MEDI4736), MEDIO680, and BMS-9365569 (anti-PDL1), MEDI6469 (anti-OX40 agonist), BMS-986016, IMP701, IMP731, IMP321 (anti-LAG3) and GITR ligand.

### AR-R17779

AR-R17779 is a drug that acts as a potent and selective full agonist for the α7 subtype of neural nicotinic acetylcholine receptors (Mullen et al. Journal of Medicinal Chemistry 43 (22): 4045-4050; Macor et al. The Journal of Organic Chemistry 69 (19): 6493-6495). Its IUPAC name is (2S)-2′H-spiro[4-azabicyclo[2.2.2]octane-2,5′-[1,3]oxazolidin]-2′-one and its chemical structure is:

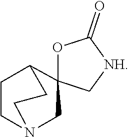 [SMILES: O=C1NC[C@@]2(CN3CCC2CC3)O1]

It has nootropic effects in animal studies (Levin et al. Behavioural Pharmacology 10 (6-7): 675-680; Van Kampen et al. Psychopharmacology 172 (4): 375-383), but its effects do not substitute for those of nicotine (Grottick et al. The Journal of Pharmacology and Experimental Therapeutics 294 (3): 1112-1119). It has also recently been studied as a potential novel treatment for arthritis (Van Maanen et al. Arthritis and rheumatism 60 (1): 114-122).

### Other ACh Pathway Agonists

In addition to AR-R17779, e.g., AR-R 17779 hydrochloride as an α7-selective agonist, and donepezil, e.g., Donepezil hydrochloride as a potent AChE inhibitor, other Ach agonists contemplated for use in the current invention include the following: 4BP-TQS (Allosteric agonist at α7 nAChR); A 582941 (Partial agonist at α7 nAChR); A 844606 (Selective α7 nAChR partial agonist); 3-Bromocytisine (Potent agonist of α4β4, α4β2 and α7 nACh receptors); DMAB-anabaseine dihydrochloride (Partial agonist at α7-containing receptors and antagonist at α4β2 neuronal nicotinic receptors); GTS 21 dihydrochloride (Partial agonist at α7 nAChR); PHA 543613 hydrochloride (Potent and selective α7 nAChR agonist); PHA 568487 (α7-selective agonist); PNU 282987 (Selective α7 nAChR agonist); S 24795 (Partial agonist at α7 nAChR); SEN 12333 (α7 nAChR agonist; histamine H3 antagonist); TC 1698 dihydrochloride (α7-selective agonist); A 85380 dihydrochloride (High affinity and selective α4β2 agonist); 3-Bromocytisine (Potent agonist of α4β4, α4β2 and α7 nACh receptors); CC4 (High affinity and subtype-selective α6β2 and α4β2 partial agonist); 5-Iodo-A-85380 dihydrochloride (High affinity α4β2 and a602 subtype-selective agonist); (−)-Nicotine ditartrate (Prototypical nAChR agonist); 3-pyr-Cytisine (High affinity, partial agonist of α4β2 receptors); RJR 2403 oxalate (α4β2 selective nicotinic agonist); SIB 1508Y maleate (Potent agonist of α4β2, a204, α4β4 and 304 nACh receptors); TC 2559 difumarate (Selective partial agonist at α4β2 receptors); Varenicline tartrate (Orally active, subtype-selective α4β2 partial agonist); A 844606 (Selective α7 nAChR partial agonist); A 85380 dihydrochloride (High affinity and selective α4β2 agonist); 4-Acetyl-1,1-dimethylpiperazinium iodide (Nicotinic agonist); 1-Acetyl-4-methylpiperazine hydrochloride (Nicotinic agonist); (+)-Anabasine hydrochloride (Neuronal nicotinic receptor partial agonist); (±)-Anatoxin A fumarate (Nicotinic agonist); 3-Bromocytisine (Potent agonist of α4β4, α4β2 and α7 nACh receptors); Carbamoylcholine chloride (Cholinergic receptor agonist); CC4 (High affinity and subtype-selective α6β2 and α4β2 partial agonist); Cisapride (5-HT4 agonist; stimulates intestinal ACh release); (−)-Cytisine (Potent, selective neuronal nicotinic agonist); DMAB-anabaseine dihydrochloride (Partial agonist at α7-containing receptors and antagonist at α4β2 neuronal nicotinic receptors); (±)-Epibatidine (High affinity nicotinic agonist); (−)-Lobeline hydrochloride (Nicotinic partial agonist); RJR 2429 dihydrochloride (Nicotinic receptor agonist); Sazetidine A dihydrochloride (α4β2 receptor ligand; may act as an agonist or a desensitizer); SIB 1553A hydrochloride (Subunit selective nAChR agonist); Tropisetron hydrochloride (Potent 5-HT3 receptor antagonist; also partial agonist of α7 nAChR); UB 165 fumarate (Subunit selective nAChR agonist); Donepezil hydrochloride (Potent AChE inhibitor); Ambenonium dichloride (Cholinesterase inhibitor); Galanthamine hydrobromide (Cholinesterase inhibitor); PE 154 (Fluorescent, potent AChE and BChE inhibitor); Phenserine (Cholinesterase inhibitor); Physostigmine hemisulfate (Cholinesterase inhibitor); Rivastigmine tartrate (Dual AChE and BChE inhibitor); and/or Tacrine hydrochloride (Cholinesterase inhibitor).

### Viral Vectors

In certain embodiments of the invention, delivery is in the form of a vector which may be a viral vector, such as a lenti- or baculo- or optionally adeno-viral/adeno-associated viral vectors, but other means of delivery are known (such as yeast systems, microvesicles, gene guns/means of attaching vectors to gold nanoparticles) and are provided. A vector may mean not only a viral or yeast system (for instance, where the nucleic acids of interest may be operably linked to and under the control of (in terms of expression, such as to ultimately provide a processed polypeptide) a promoter), but also direct delivery of nucleic acids into a host cell. While in herein methods the vector may be a viral vector and this is optionally an AAV, other viral vectors as herein discussed can be employed, such as lentivirus. For example, baculoviruses may be used for expression in insect cells. These insect cells may, in turn be useful for producing large quantities of further vectors, such as AAV or lentivirus vectors adapted for delivery of the present invention. Also envisaged is a method of delivering the present SOCS3 nucleic acid/polypeptide comprising delivering to a cell mRNA encoding the SOCS3 polypeptide. It will be appreciated that in certain embodiments the SOCS3 sequence can be truncated, and/or comprised of less than 225 amino acids, and/or is a nuclease or nickase, and/or is codon-optimized, and/or comprises one or more mutations, and/or comprises a chimeric SOCS3-containing protein, and/or the other options as herein discussed. In certain embodiments, AAV or lentiviral vectors are employed.

Use of an AAV of any known serotype is contemplated, including, e.g., AAV-1, AAV-2, AAV-3, AAV-4, AAV-5, AAV-6, AAV-7, AAV-8, AAV-9, and any other serotypes and/or hybrid forms of one or more of the preceding serotypes.

In certain embodiments, a viral vector of the invention expresses a polypeptide (e.g., a SOCS3 polypeptide, e.g., full-length, truncated and/or modified forms thereof) globally. Alternatively, the polypeptide is expressed in a cell-type and/or tissue-specific manner, as described in additional detail below.

As noted above, the nucleic acids of the invention (and therefore the SOCS3 polypeptide sequences therein encoded) can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual, and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed.

The viral vectors can be injected into the tissue of interest. For cell-type specific and/or tissue-specific delivery and/or expression of SOCS3, the expression of SOCS3 can be driven by a cell-type specific and/or tissue-specific (e.g., neuron-specific, etc.) promoter. For example, liver-specific expression might use the Albumin promoter and neuron-specific expression might use the Synapsin I promoter.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10³-1×10¹¹ particles or about 1×10-1×10¹² particles), and most preferably at least about 1×10⁰ particles (e.g., about 1*×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹² particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹⁶ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The doses herein are based on an average 70 kg individual. The frequency of administration is within the ambit of the medical or veterinary practitioner (e.g., physician, veterinarian), or scientist skilled in the art.

### Lentivirus

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In certain embodiments, minimal non-primate lentiviral vectors based on the equine infectious anemia virus (EIAV) are also contemplated, especially, e.g., for ocular therapies (see, e.g., Balagaan, J Gene Med 2006; 8: 275-285, Published online 21 Nov. 2005 in Wiley InterScience (www.interscience.wiley.com). DOI: 10.1002/jgm.845).

### Compound Libraries

In certain embodiments, libraries of compounds (e.g., small molecules, though peptide and nucleic acid libraries are also contemplated) can be used to screen for inducers of SOCS3 expression. Such compound libraries, also referred to as collections of compounds, are produced according to methods known in the art, with many examples commercially available. According to different embodiments of the invention, the small molecule collection of compounds that is screened includes at least 100, at least 1000, at least 2,000, at least 10,000, at least 50,000 or at least 100,000 compounds. Any subranges are also included within the scope of the invention.

### Retrovirus-Mediated Delivery of Modified SOCS3

As used herein, the term “retrovirus” refers an RNA virus that reverse transcribes its genomic RNA into a linear double-stranded DNA copy and subsequently covalently integrates its genomic DNA into a host genome.

Retroviruses are a common tool for gene delivery (Miller, 2000, Nature. 357: 455-460). Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses that are also useful as a gene delivery vehicle or vector. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one aspect, HIV based vector backbones (i.e., HIV cis-acting sequence elements) are preferred.

Retroviral vectors and more particularly lentiviral vectors may be used in practicing the present invention. Accordingly, the term “retrovirus” or “retroviral vector”, as used herein is meant to include “lentivirus” and “lentiviral vectors” respectively.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In one aspect, a vector is a gene delivery vector. In one aspect, a vector is used as a gene delivery vehicle to transfer a gene into a cell.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In one aspect, a hybrid vector refers to a vector or transfer plasmid comprising retroviral e.g., lentiviral, sequences for reverse transcription, replication, integration and/or packaging.

In particular aspects, the terms “lentiviral vector”, “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention.

Modified SOCS3 mRNA Delivery

It is contemplated that modified SOCS3 mRNAs, optionally attached to cell-penetrating peptides or similar agents for achieving cytoplasmic localization, can be used to provide additional expression of SOCS3 in vivo. Modified mRNA delivery approaches have been previously described in, e.g., WO2013151666, WO2013106496, WO2013101690 and US2013259924, the contents of which are incorporated by reference in their entireties.

### Cell Culture

Candidate SOCS3 modulatory agents of the invention can be tested for SOCS3 induction activity in cell culture using cell lines such as B16F10-Luciferase Melanoma cell line, Lewise lung carcinoma cells, or other mammalian cells (e.g., human cell lines HepG2, Hep3B, HeLa, DU145, Calu3, SW480, T84, PL45, etc., and mouse cell lines LMTK-, Hepal-6, AML12, Neuro2a, etc.) to determine the extent of impact of test compounds upon SOCS3 levels.

### Pharmaceutical Compositions

The compositions of the invention (e.g., antibody-SOCS3 fusion polypeptides and the nucleic acid molecules encoding them, small molecule, peptide or other inducers of SOCS3, etc.) can be administered in a pharmaceutically acceptable excipient, such as water, saline, aqueous dextrose, glycerol, or ethanol. The compositions can also contain other medicinal agents, pharmaceutical agents, adjuvants, carriers, and auxiliary substances such as wetting or emulsifying agents, and pH buffering agents. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a disease characterized by cell growth, or otherwise reduce the pathological consequences of tumor growth/metastasis/tumor burden. In another embodiment, an effective amount is an amount sufficient to inihibit the proliferation or growth of an undesirable cell type (e.g. a tumor). Alternatively, an effective amount is an amount sufficient to induce elevation of SOCS3 levels in a target cell type (e.g., a host tumor or tumor-associated tissue, such as vascular and/or neuronal tissues). A therapeutically effective amount can be provided in one or a series of administrations. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the agent (e.g., compound, peptide, antibody, etc.) being administered.

The dosage of the modified SOCS3 fusion protein compositions and/or inducers of SOCS3 (e.g., flavanones) of the invention can vary from about 0.01 mg/m² to about 500 mg/m², preferably about 0.1 mg/m² to about 200 mg/m², most preferably about 0.1 mg/m² to about 10 mg/m². Alternatively, the dosages of the modified SOCS3 fusion protein compositions and/or inducers of SOCS3 (e.g., flavanones) can vary from about 0.01 mg/kg per day to about 1000 mg/kg per day. It is expected that doses ranging from about 50 to about 2000 mg/kg will be suitable. In various embodiments, a dosage ranging from about 0.5 to about 100 mg/kg of body weight is useful; or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 5 mg/kg/day, 25 mg/kg/day and 75 mg/kg/day).

Administrations can be conducted infrequently, or on a regular weekly basis until a desired, measurable parameter is detected, such as diminution of disease symptoms. Administration can then be diminished, such as to a biweekly or monthly basis, as appropriate.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with tumoricidal antibodies. Therapeutic compositions of modified SOCS3 fusion protein compositions and/or inducers of SOCS3 (e.g., flavanones) can be administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are optionally supplied in unit dosage form suitable for administration of a precise amount. Also contemplated by this invention are slow release or sustained release forms, whereby a relatively consistent level of the active compound are provided over an extended period.

Another method of administration is intratumoral or in the immediate vicinity of a tumor, for instance by direct injection directly into the tumor tissue site; into a site that requires inhibition of cell growth; or into a site where a cell, tissue or organ is at risk of tumor formation and/or growth. Alternatively, the composition of the invention is administered systemically. For methods of combination therapy comprising administration of a composition of the invention in combination with a chemotherapeutic agent, the order in which the compositions are administered is interchangeable. Concomitant administration is also envisioned.

Methods of the invention are particularly suitable for use in inhibiting tumor growth or proliferation in melanoma, lung and other tumors characterized by well-defined tissue beds (e.g., surrounding vascular, neuronal and/or muscle tissue(s)). Direct injection of a therapeutic SOCS3 and/or SOCS3 inducing agent is an option for delivery to many such tumors and/or to the vicinity of such tumors.

In instances where the site of delivery is the brain, the therapeutic agent must be capable of being delivered to the brain. The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may require the use of specific drug delivery strategies. Delivery of therapeutic agents to the CNS can be achieved by several methods.

One method relies on neurosurgical techniques. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents, such as leukotrienes or by convention enhanced delivery by catheter (CED). Further, it may be desirable to administer the compositions locally to the area in need of treatment; this can be achieved, for example, by local infusion during surgery, by injection, by means of a catheter, or by means of an implant, the implant being of a porous, non-porous, or gelatinous material, including membranes, such as silastic membranes, or fibers. A suitable such membrane is Gliadel® provided by Guilford Pharmaceuticals Inc.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer-based systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

A chimeric polypeptide (e.g., SOCS3-dAb fusion protein) disclosed herein may be derivatized by the attachment of one or more chemical moieties to the protein moiety. The chemically modified derivatives may be further formulated for intraarterial, intraperitoneal, intramuscular, subcutaneous, intravenous, oral, nasal, rectal, buccal, sublingual, pulmonary, topical, transdermal, or other routes of administration. Chemical modification of biologically active proteins has been found to provide additional advantages under certain circumstances, such as increasing the stability and circulation time of the therapeutic protein and decreasing immunogenicity. The chemical moieties suitable for derivatization may be selected from among water soluble polymers. The polymer selected should be water soluble so that the protein to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/polypeptide conjugate will be used therapeutically, and if so, the desired dosage, circulation time, resistance to proteolysis, and other considerations.

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols and polyvinyl alcohol. Polyethylene glycol propionaldenhyde may provide advantages in manufacturing due to its stability in water.

The polymer may be of any molecular weight, and may be branched or unbranched. In one embodiment, the polymer is polyethylene glycol having a preferred molecular weight between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional activity of the protein. In one example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues, those having a free carboxyl group may include aspartic acid residues glutamic acid residues and the C-terminal amino acid residue. Sulfhydry groups may also be used as a reactive group for attaching the polyethylene glycol molecule(s). Preferred for therapeutic purposes is attachment at an amino group, such as attachment at the N-terminus or lysine group.

### Use

The methods of the invention provide a means for inhibiting tumor growth or for otherwise reducing or preventing oncogenic cell proliferation. This inhibiting can be carried out in vivo or in vitro. For therapeutic uses in vivo, the compositions or agents described herein may be administered systemically, for example, formulated in a pharmaceutically-acceptable buffer such as physiological saline. The compositions and methods of the invention can be used for the treatment of virtually any condition in which the administration of an anti-proliferative and/or anti-cancer drug is useful. Such conditions include neoplastic diseases or disorders such as breast cancer, melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, etc. In certain embodiments, the compositions of the invention are administered in a form that provides for their delivery across the blood-brain barrier. In the context of treating a neoplasia associated with the CNS, a modified SOCS3 fusion protein of the invention and/or inducer of SOCS3 is provided in an amount sufficient to inhibit tumor cell growth, enhance apoptosis, or reduce a symptom associated with the growth of a tumor and/or tumor cells. Typically, the compositions are administered to a patient already suffering from a disease or disorder characterized by neoplasia (e.g., a tumor), in an amount sufficient to cure or at least partially arrest a symptom associated with tumor growth and/or metastasis.

For in vitro uses, cells in culture (e.g., oncogene cell lines, neural cells, vascular cells, muscle cells) are contacted with a modified SOCS3 fusion protein of the invention and/or inducer of SOCS3 of the invention in an amount sufficient to inhibit growth of the cell population in vitro. A cell in vitro that is contacted with a modified SOCS3 fusion protein of the invention and/or inducer of SOCS3 of the invention is less likely to undergo growth than a cell cultured under similar conditions but not contacted with a modified SOCS3 fusion protein of the invention and/or inducer of SOCS3. Advantageously, a modified SOCS3 fusion protein of the invention and/or an inducer of SOCS3 of the invention reduces or prevents the growth or proliferation of cultured cells and inhibit/reduce the in vitro expansion of the cultured cells. Optionally, the cultured cells in combination with a modified SOCS3 fusion protein of the invention and/or an inducer of SOCS3 are administered to a patient in need thereof.

### Combination Therapies

As described herein, modified SOCS3 fusion protein compositions and/or inducers of SOCS3 (e.g., flavanones) of the invention are useful for reducing tumor growth or otherwise preventing proliferation. Accordingly, the compositions of the invention may, if desired, be combined with any standard therapy typically used to treat a disease or disorder characterized by excess cell growth. In one embodiment, the standard therapy is useful for the treatment of cell growth associated with breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

For the treatment of diseases or disorders affecting the central nervous system, the modified SOCS3 fusion protein compositions and/or inducers of SOCS3 (e.g., flavanones) are provided in combination with agents that enhance transport across the blood-brain barrier. Such agents are known in the art and are described, for example, by U.S. Patent Publication Nos. 20050027110, 20020068080, and 20030091640. Other compositions and methods that enhance delivery of an active agent across the blood brain barrier are described in the following publications: Batrakova et al., Bioconjug Chem. 2005 July-August; 16(4):793-802; Borlongan et al., Brain Res Bull. 2003 May 15; 60(3):297-306; Kreuter et al., Pharm Res. 2003 March; 20(3):409-16; and Lee et al., J Drug Target. 2002 September; 10(6):463-7. Other methods for enhancing blood-brain barrier transport include the use of agents that permeabilize tight junctions via osmotic disruption or biochemical opening; such agents include RMP-7 (Alkermes), and vasoactive compounds (e.g., histamine). Other agents that enhance transport across the blood-brain barrier enhance transcytosis across the endothelial cells to the underlying brain cells. Enhanced transcytosis can be achieved by increasing endocytosis (i.e. internalisation of small extracellular molecules) using liposomes or nanoparticles loaded with a drug of interest.

Alternatively, a modified SOCS3 fusion protein compositions and/or inducers of SOCS3 (e.g., flavanones) or other composition of the invention is administered in combination with a chemotherapeutic, such that the modified SOCS3 fusion protein compositions and/or inducers of SOCS3 (e.g., flavanones) complements the chemotherapy agent. For example, a patient that receives a chemotherapeutic and a modified SOCS3 fusion protein compositions and/or inducers of SOCS3 (e.g., flavanones) of the invention is more likely to exhibit reduced or prevented tumor growth than a patient that receives only the chemotherapeutic. A composition of the invention is administered prior to, concurrent with, or following the administration of any one or more of the following: a chemotherapeutic agent, radiation agent, hormonal agent, biological agent, an anti-inflammatory agent. Exemplary chemotherapeutic agents include tamoxifen, trastuzamab, raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, and vincristine.

### Patient Monitoring

The treatment or disease state of a patient administered a composition of the invention that includes a chimeric polypeptide can be monitored by assessing the level of growth in a cell, tumor, tissue, or organ of the patient. For patients suffering from a disease or disorder characterized by neoplasia, this monitoring typically involves monitoring tumor size via imaging or biopsy, but also may involve monitoring of associated phenotypes, including, e.g., neurological symptoms. Neurological symptoms may include any one or more of the following: tremors; rigidity; substantia nigra impairment; depression; areflexia; hypotonia; fasciculations; muscle atrophy; involuntary movements of the head, trunk and limbs; mutated survival motor neuron 1 (SMN1) gene; sudden numbness or weakness; sudden confusion; sudden trouble speaking; sudden trouble understanding speech; sudden trouble seeing in one or both eyes; sudden trouble with walking; dizziness; loss of balance; loss of coordination; sudden severe headache of unknown etiology; bradykinesia; postural instability; loss of consciousness; confusion; lightheadedness; dizziness; blurred vision; tired eyes; ringing in the ears; bad taste in the mouth; fatigue; lethargy; an alteration in sleep pattern; behavioral alteration; mood alteration; memory deficit; concentration deficits; attentional deficits; cognitive deficits; vomiting; nausea; convulsions; seizures; inability to awaken; pupil dilation; slurred speech; weakness or numbness in the extremities; restlessness; and agitation. Compositions that produce a reduction in the severity of any one or more of the preceding symptoms are considered useful in the methods of the invention.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

### Example 1: Expression of SOCS3 in Host Tissues Suppresses Pathologic Angiogenesis, Tumor Growth and Metastasis

SOCS3 was recently demonstrated to be angiostatic, and SOCS3 deletion was shown to increase neovascularization in pathologic conditions. Systemic deletion of Socs3 was also previously demonstrated to be embryonic lethal (Marine, 1999).

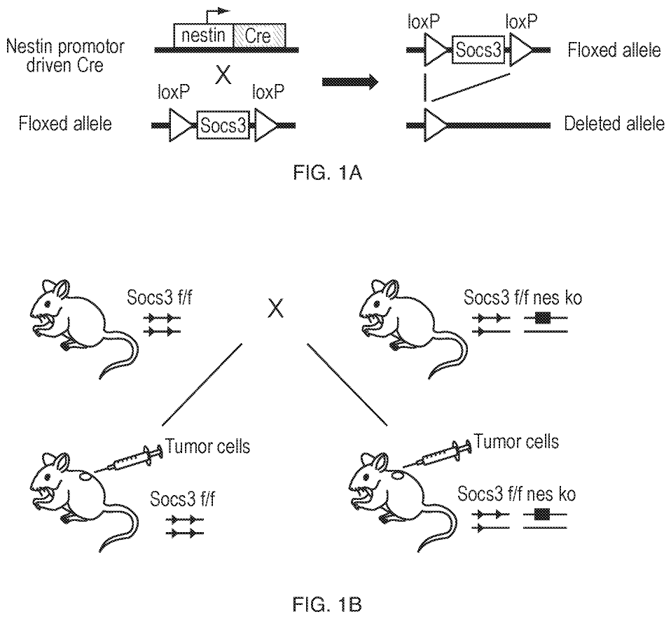

To examine the role of SOCS3 in development and pathologic angiogenesis in mouse models of oxygen-induced retinopathy (OIR) and cancer, a Cre/Lox system was used to achieve deletion of SOCS3 in host vessels and neurons (FIG. 1A). The specific targeting strategy employed for the generation of conditional knockout mice of Socs3 used neuronal-specific nestin Cre (Socs3^{flox/flox}/nestin-Cre). To test the impact of conditional knockout of Socs3, such mice were implanted subcutaneously with mouse tumor carcinoma cells, before assessing tumor growth in the murine model system (FIG. 1B).

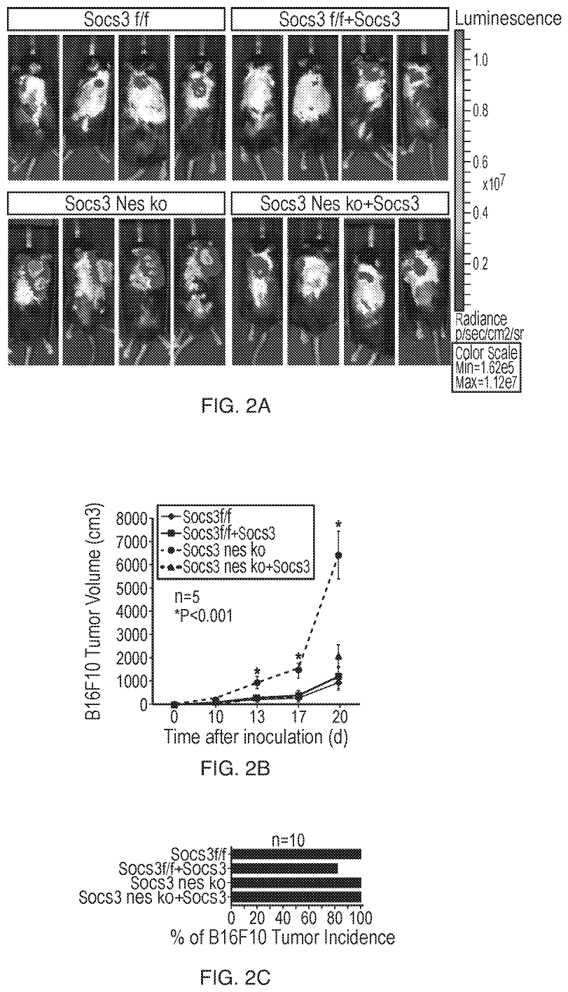

Neuronal deficiency of Socs3 promoted melanoma growth, whereas exogenous Socs3 (introduced via lentivirus, also termed “lenti-Socs3”) inhibited melanoma growth (FIGS. 2A-2C). In such experiments, 1×10⁵ cells of B16F10-Luciferase Melanoma cell line were implanted into Socs3f/f and Socs3 Nestin ko mice with or without lenti-Socs3 sub-cutaneous (S.Q.) injection (4 days before tumor cell injection). Tumors were imaged after luciferin i.p. injection, using an imaging system, and tumor growth curves were measured. Tumors grew much bigger in Socs3 nestin-conditional knockout mice than in Socs3 f/f (wild-type) mice, and preinjected Lenti-Socs3 was observed to be capable of blocking the promotion of tumor growth seen with neuronal deficiency of Socs3. Indeed, lenti-Socs3 preinjection was observed to block tumor cell seeding entirely in 20% of wild type mice (FIG. 2C). Thus, presence of Socs3, whether natively or by lentiviral overexpression had a melanoma tumor growth-inhibiting effect.

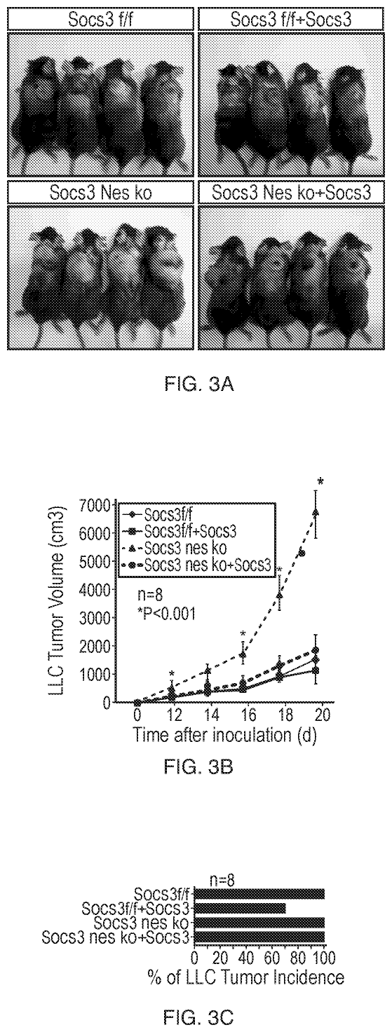

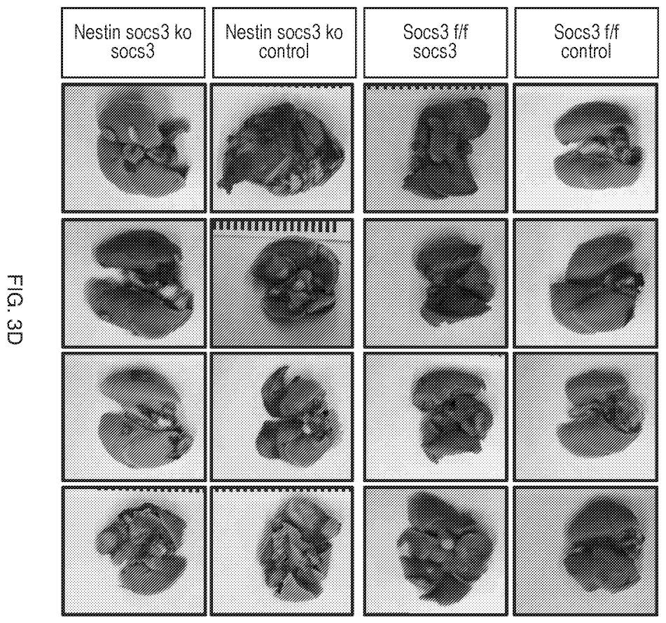

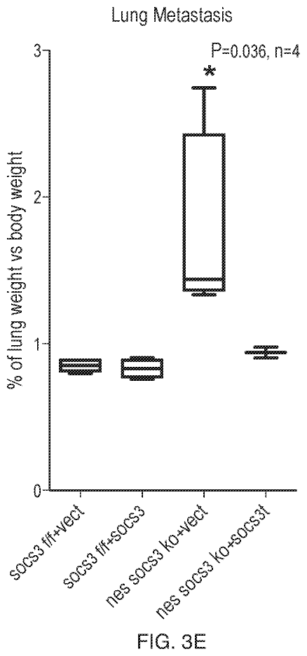

To examine whether the observed tumor growth-inhibiting effect of Socs3 was applicable to other forms of tumor, experiments similar to those performed upon melanoma-injected mice were also performed upon lung carcinoma-injected mice. As shown in FIGS. 3A-3C, neuronal deficiency of Socs3 was observed to have promoted lung carcinoma growth, whereas exogenously introduced Socs3 (lenti-Socs3) inhibited lung carcinoma growth. In such experiments, 1×10⁶ cells of Lewis lung carcinoma cell line were implanted into Socs3f/f and Socs3 Nestin ko mice with or without lenti-Socs3 administered by sub-cutaneous injection (4 days before tumor cell injection). Tumors were then imaged and tumor growth curves were measured and plotted. Tumors grew much bigger in Socs3 nes knockout mice than in Socs3 f/f (wild-type for the Socs3 gene). Preinjection of lenti-Socs3 was again observed to block the promotion of tumor growth otherwise caused by neuronal deficiency of Socs3 (FIGS. 3A-3B). Indeed, lenti-Socs3 preinjection was shown to prevent tumor cell seeding in 25% of wild type mice (FIG. 3C). In addition to tumor growth, Socs3 was also identified to impact the number and size of lung matastases in tumor-bearing mice. As shown in FIG. 3D and summarized in graph format in FIG. 3E, bouin's staining of lung revealed a dramatic elevation of lung metastases in Socs3 conditional knockout mice, which was successfully reduced to near-wild-type levels upon administration of additional Socs3 (lenti-Socs3 injection). Thus, a role for Socs3 (and therefore Socs3 modulating agents) in metastasis prevention was also established.

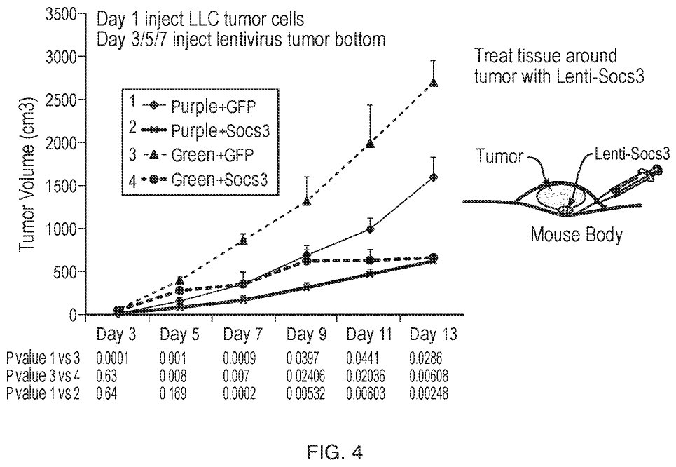

The lung carcinoma growth-inhibitory effect of lenti-Socs3 administration was then assessed across repeated administrations. As shown in FIG. 4, lenti-Socs3 treatment inhibited lung carcinoma growth on Socs3 f/f and SX nes knockout mice, when administered at days 3, 5 and 7 post-injection of Lewis lung carcinoma (LLC) tumor cells. In two instances (represented by the circle and “x” data in FIG. 4), hSOCS3 was injected 4 days prior to tumor inoculation in order to determine whether pre-treatment of the microenvironment would have an effect on tumor inhibition following injection of the tumor cells. By treating the host and partially treating tumors with lentivirus over-expressing Socs3, significant suppression of the tumor growth induced by Socs3 neuronal deficiency (in the absence of Socs3 overexpression) was again observed. Indeed, a complete prevention of tumor growth was observed between days 9 and 13 in Socs3 f/f (wild-type) mice administered lenti-Socs3 at days 3, 5 and 7 (FIG. 4). Furthermore, the results provided in FIG. 4 show that pre-treatment of the microenvironment and subsequent treatment with Socs3 resulted in lung cancer tumor stasis.

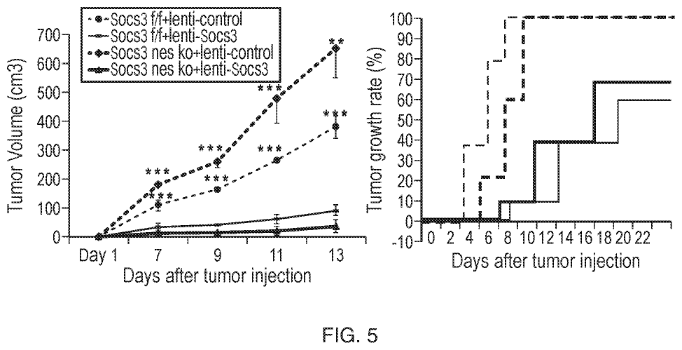

Lung carcinoma tumor growth rates were examined over an extended duration, as shown in FIG. 5. In FIG. 5, exogenous Socs3 (referred to as lenti-Socs3 in FIG. 5) was observed to have inhibited lung carcinoma growth in Socs3 f/f and Socs3 nes knockout mice (when tissue around tumor and tumor cells was treated with lenti-Socs3). In such experiments, lenti-Socs3 treated Lewis lung carcinoma (LLC) cells were implanted with lenti-Socs3 to treat the Socs3 f/f and Socs3 nes knockout mice, and the tumor growth curves were measured. Exogenous Socs3 in tumor cells and host dramatically reduced lung carcinoma growth and seeding, as compared to control mice not administered exogenous Socs3. Remarkably, as demonstrated in FIG. 5, 30-40% of Socs3 f/f and Socs3 nes knockout mice did not form tumor after over 20 days post-LLC tumor cell implantation.

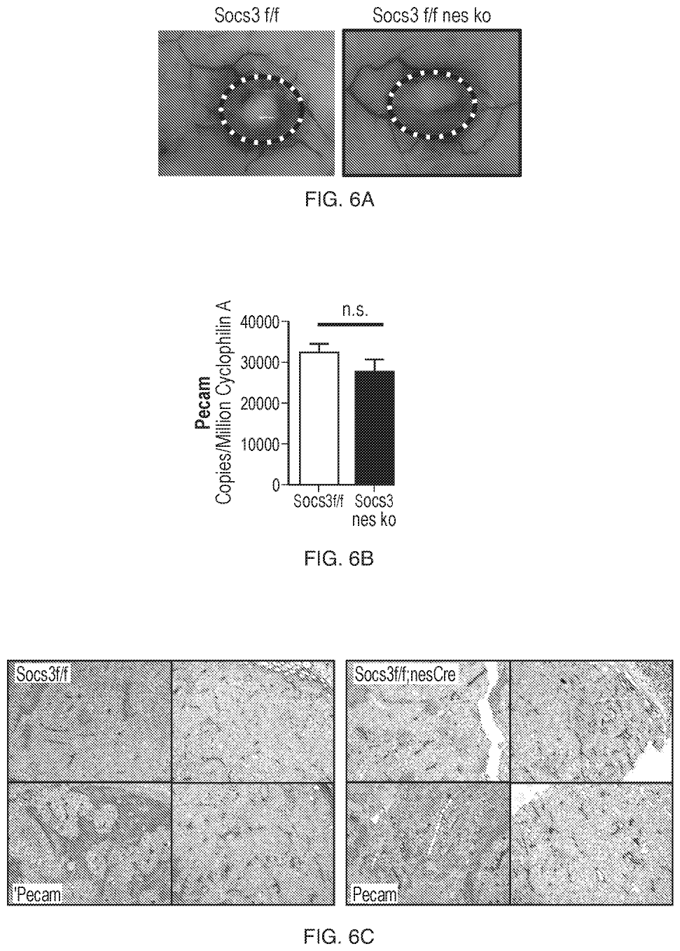

Blood vessels surrounding and associated with tumors were also examined for effect of Socs3 knockout and exogenous lenti-Socs3 administration. As shown in FIGS. 6A-6C, blood vessel around and inside the tumor on Socs3 f/f and Socs3 nes knockout mice were similar. Specifically, the blood vessels around similarly sized tumors of wild-type and knockout mice were physiologically observed to be similar, while there was also no difference in Pecam mRNA levels (a blood vessel marker) detected inside of tumors when wild-type and knockout mice were compared (FIG. 6B). Consistent with this Pecam mRNA observation, the corresponding density of Pecam immunohistochemistry staining of tumors from wild-type and knockout mice was also similar (FIG. 6C).

Thus, conditional loss of SOCS3 was observed to lead to increased pathologic neovascularization, resulting in pronounced retinopathy and increased tumor size (FIGS. 2-6). In contrast, physiologic vascularization was not regulated by SOCS3 (FIG. 6A). In vitro, SOCS3 knockdown increased proliferation and sprouting of endothelial cells co-stimulated with IGF-1 and TNFα via reduced feedback inhibition of the STAT3 and mTOR pathways. Together, these results identified SOCS3 as a pivotal endogenous feedback inhibitor of pathologic angiogenesis.

Local overexpression of SOCS3 in the host using lentiviral vectors prior to tumor implantation in SOCS3 conditional knockout mice then confirmed SOCS3 as a therapeutic target acting at the converging crossroads of growth factor and cytokine-induced vessel and tumor growth. A 4.5-fold reduction in growth of both Lewis lung carcinoma (LLC) and B16F10 melanoma in treated mice (lentivirus containing SOCS3), as compared to lentivirus alone was observed (FIG. 2A to FIG. 6). Despite equal tumor cell seeding, 20-25% of mice overexpressing SOCS3 never developed tumors over a period of 30 days, whereas all control animals grew large tumors (FIG. 5). In addition, conditional deletion of SOCS3 significantly increased lung metastasis, which was then shown to be reduced by reintroducing SOCS3 using lentivirus.

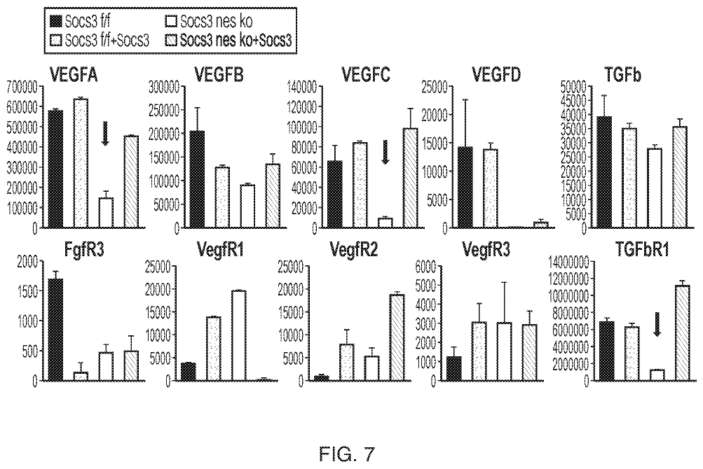

The impact upon pro-angiogenic growth factors of nestin-conditional Socs3 deletion and restoration of Socs3 via lentiviral overexpression was also examined. As shown in FIG. 7, a number of pro-angiogenic growth factors and receptors (particularly VEGFA, VEGFC and TGFβR1) were down-regulated rather than up-regulated in tumor obtained from Socs3 nes knockout mice. Meanwhile, such levels were generally restored when Socs3 was overexpressed in such conditional knockout mice using lentivirus. Notably, the above experiments were performed in mice having uncompromised immune responses, i.e., the mice were not SCID (severe combined immunodeficiency) mice.

### Example 2: Neural Impact of Conditional SOCS3 Deletion and Lentiviral SOCS3 Overexpression and Role of Cytokines

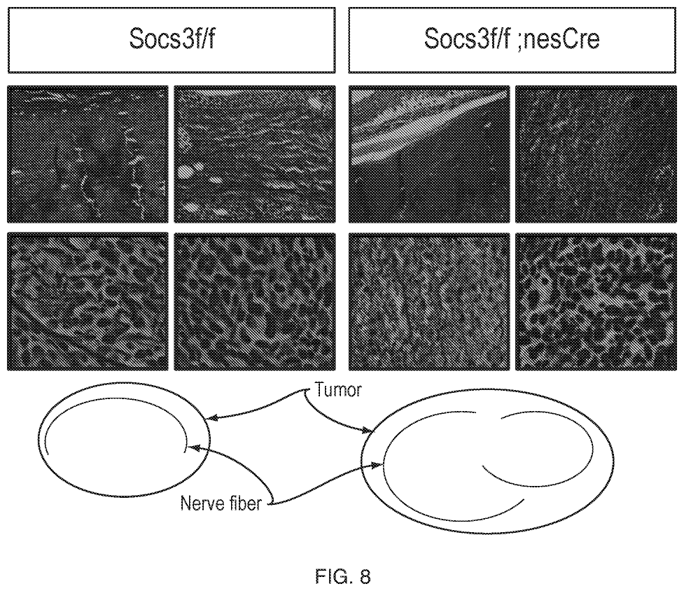

The neural impact of nestin-conditional Socs3 knockout and lentiviral overexpression of Socs3 (either in conditional Socs3 knockout mice or in mice wild-type for Socs3) was also examined. As shown in FIG. 8, Bodian staining was performed upon nerve fiber associated with tumors from Socs3 f/f and Socs3 nestin-conditional knockout mice, which demonstrated that more nerve fiber was observed in knockout mice than in wild-type mice; however, the nerve fibers of knockout mice appeared thinner, indicating that these were unhealthy nerve fibers that promoted tumor growth.

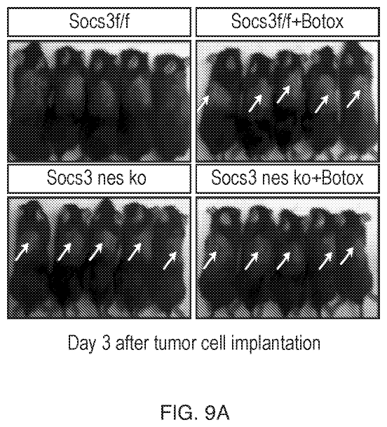

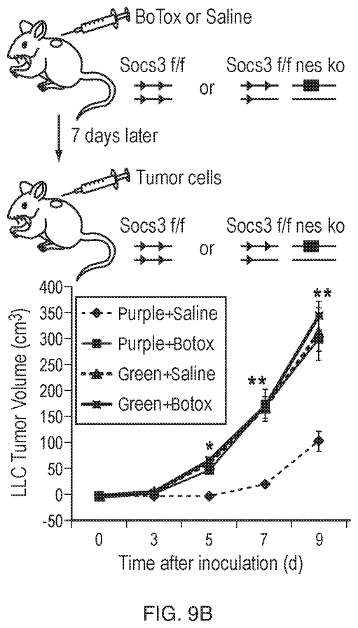

In view of the above results, the impact of the neuron axonal ACh-release blocking agent, Botulinum toxin (Botox), upon tumor growth was examined. As shown in FIGS. 9A and 9B, Botox injection (0.25 U/20 g mice) 7 days before tumor cell injection promoted tumor seeding, indicating that dysfunction of motor neurons promoted tumor seeding. Thus, Botox injection promoted tumor seeding, indicating that dysfunction of motor neuron promoted tumor seeding. As shown in FIG. 9B, Botox injection to wild-type mice effectively mimicked the tumor growth effect of nestin-conditional Socs3 knockout in tested mice, whereas Botox had no additional effect on tumor growth when injected into nestin-conditional Socs3 knockout mice.

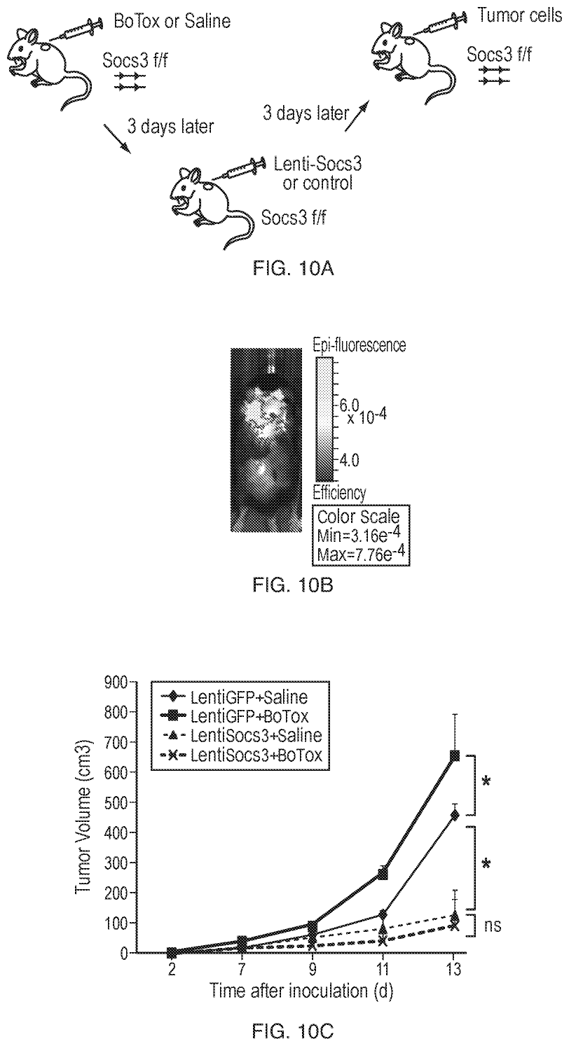

The neural effects of lentiviral Socs3 injection were also examined. As shown in FIGS. 10A-10C, lentiviral Socs3 injection following Botox injection but before tumor cell injection in wild-type Socs3 mice blocked the effect of Botox injection upon tumor engraftment/growth, indicating that Botox and Socs3 worked through similar mechanism. In order to study the relationship between the regulation of tumor growth by Botox and Socs3 deficiency, Botox was injected into the mice first and 3 days later lenti-socs3 were injected at the same spots. 3 days later, tumor cells were then implanted at the same spot of injection. Remarkably, lenti-Socs3 blocked the regulation of Botox on tumor growth, indicating that Botox and Socs3 were working through a similar mechanism.

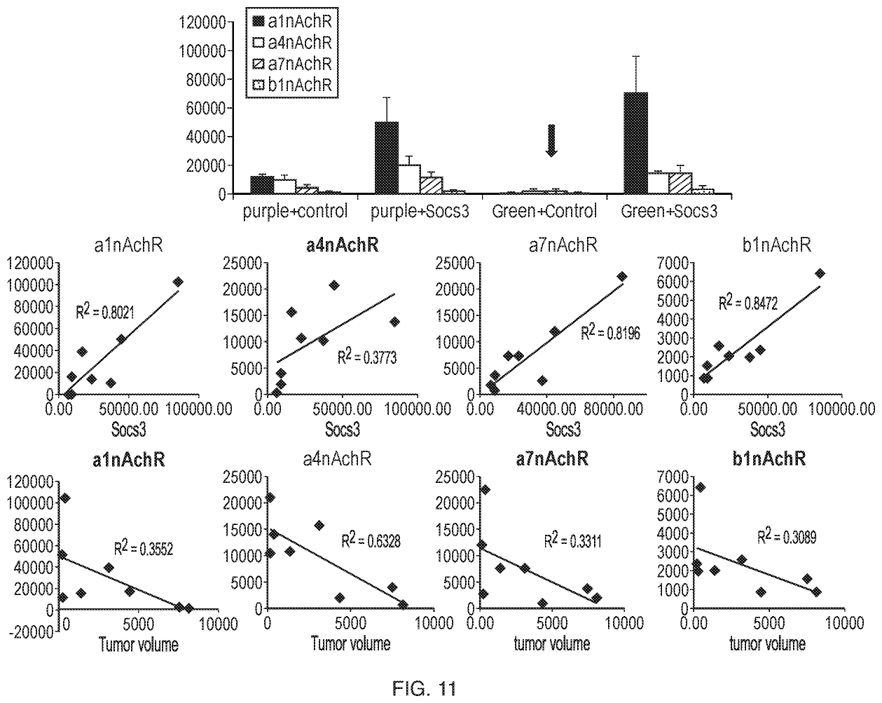

Botox acts upon acetylcholine release within neurons. To examine whether nestin-conditional Socs3 knockout could have impact similar to Botox, associations between AChR levels and Socs3 levels were examined. As shown in FIG. 11, a negative impact of nestin-conditional Socs3 knockout upon acetylcholine receptor (AChR) levels was observed (in the Figures, “purple” indicates mice carrying wild-type Socs3 alleles, “Green” corresponds to mice harboring Socs3 knockouts and “+Socs3” indicates lentiviral overexpression of Socs3). Strong positive correlations were observed between quantitative levels of Socs3 and alnAChR, α7nAChR and blnAChR, while a correspondingly negative correlation was observed between tumor volume and a4nAChR levels (FIG. 11).

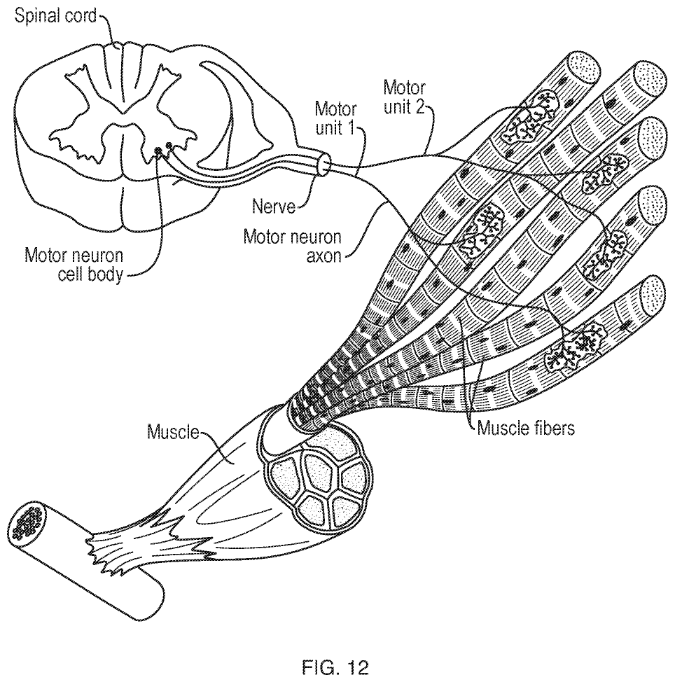

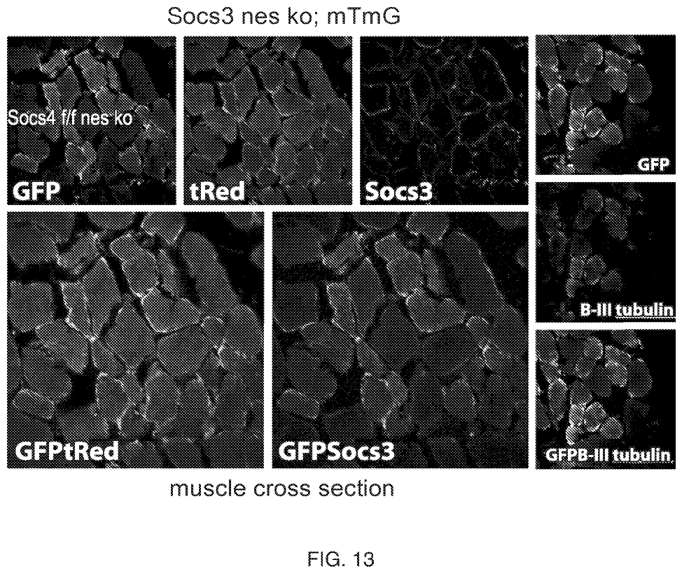

In view of the physiological interaction of motor neurons and muscle fibers (FIG. 12), the impact of Socs3 knockout was also examined in muscle fibers. As shown in FIG. 13, muscle cross-section images were obtained for a double-fluorescent Cre reporter that had been combined with the nestin-conditional Socs3 knockout. In cells in which Socs3 expression was knocked out, GFP was expressed. Such GFP expression was also observed to coincide with expression of beta III tubulin (FIG. 13).

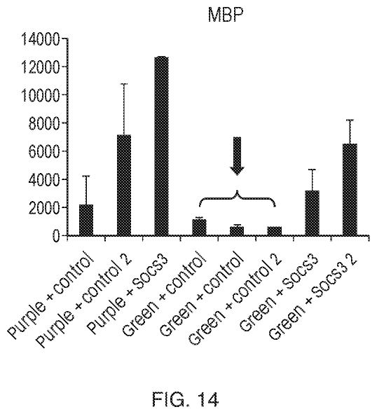

As shown in FIG. 14, nestin-conditional Socs3 knockout reduced myelin basic protein (MBP) levels (in FIG. 14, “purple” indicates mice carrying wild-type Socs3 alleles, “Green” corresponds to mice harboring Socs3 knockouts and “+Socs3” indicates lentiviral overexpression of Socs3). Myelin basic protein (MBP) is a protein believed to be important in the process of myelination of nerves in the nervous system. The level of MBP has been observed to be high (>4 ng/ml) in patients with malignant tumors, but in those who showed a good response to chemotherapy and/or radiation, it decreased or returned to the normal level. Demyelination is the loss of the myelin sheath insulating the nerves, and is the hallmark of some neurodegenerative autoimmune diseases. The immune system may play a role in demyelination associated with such diseases, including inflammation causing demyelination by overproduction of cytokines via upregulation of tumor necrosis factor or interferon. Acetylcholinesterase inhibitors may improve myelin integrity. Thus, myelin levels are an important indicator of neural health and potential neural impacts upon tumor growth.

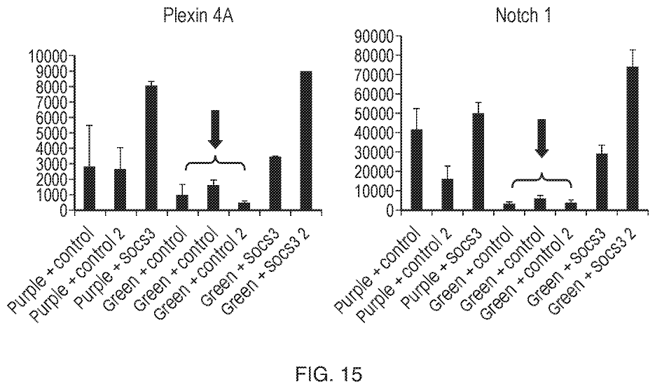

Whether nestin-conditional Socs3 knockout and lentiviral overexpression of Socs3 also impacted levels of candidate tumor suppressor genes Plexin 4A and Notch 1 was also examined. As shown in FIG. 15, nestin-conditional Socs3 knockout resulted in reduced levels of both Plexin 4A and Notch 1 (in FIG. 15, “purple” indicates mice carrying wild-type Socs3 alleles, “Green” corresponds to mice harboring Socs3 knockouts and “+Socs3” indicates lentiviral overexpression of Socs3). Plexin-A4 is a receptor for sema6A and sema6B and associates with neuropilins to transduce signals of class-3 semaphorins. Plexin A4 promotes tumor progression and tumor angiogenesis by enhancement of VEGF and bFGF signaling. Notch was originally identified as an oncogene, but recent studies have also demonstrated tumor suppressive effects for Notch receptors, illustrating the highly context-dependent nature of the pathway. The first conclusive evidence showing that Notch1 acts as a tumor suppressor came from studies in skin. Thus, Notch1 likely functions as a tumor suppressor in human skin cancers as well. Multiple components of the Notch signaling pathway, including NOTCH1, NOTCH2, and JAGGED1, show reduced expression in human basal cell carcinoma samples. Thus, modulation of Socs3 levels via knockout or lentiviral overexpression impacted Plexin 4A and Notch 1 levels.

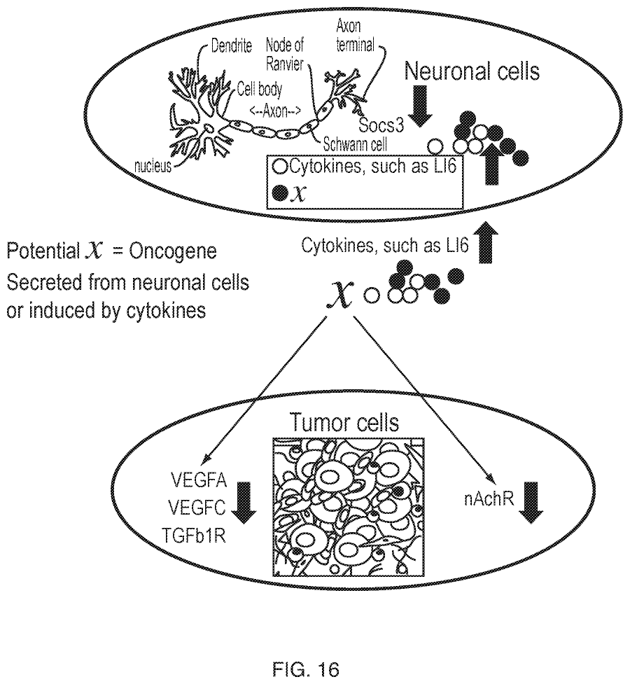

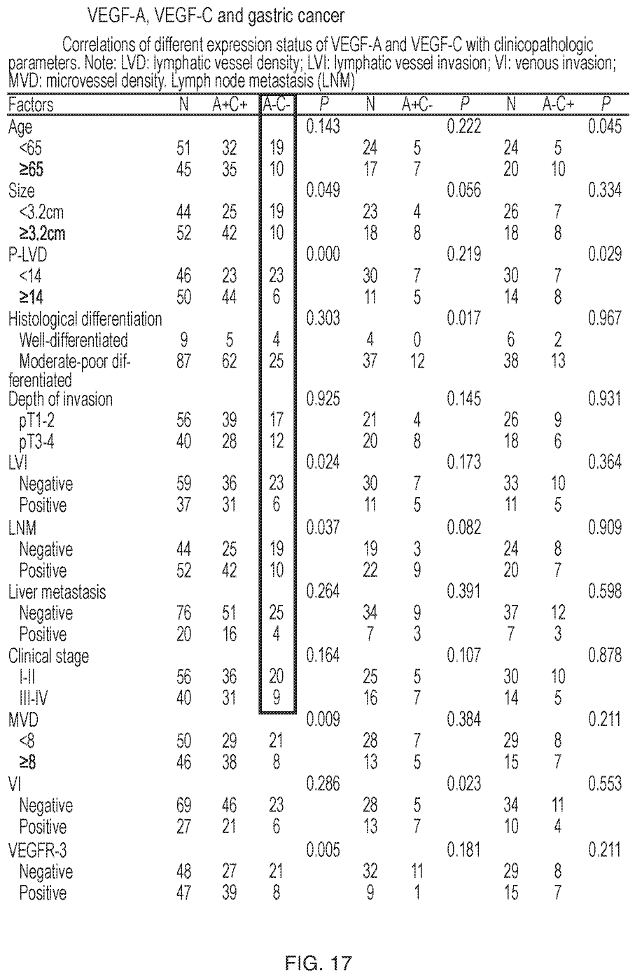

A model by which reduced expression of Socs3 in neuronal cells could have resulted in increased cytokine (e.g., LI6) expression/release is shown in FIG. 16, where an “X” factor oncogene that is secreted from neuronal cells or induced by cytokines is posited, which, in turn, would lower VEGFA, VEGFC, TGFb1R and nAChR levels, resulting in enhanced tumor cell engraftment and growth. Levels of cytokines VEGF-A and VEGF-C were correlated with clinicopathologic parameters for gastric cancer (FIG. 17).

### Example 3: Flavone/Flavanone (Naringenin)-Induced Up-Regulation of Socs3 Significantly Decreased Tumor Growth

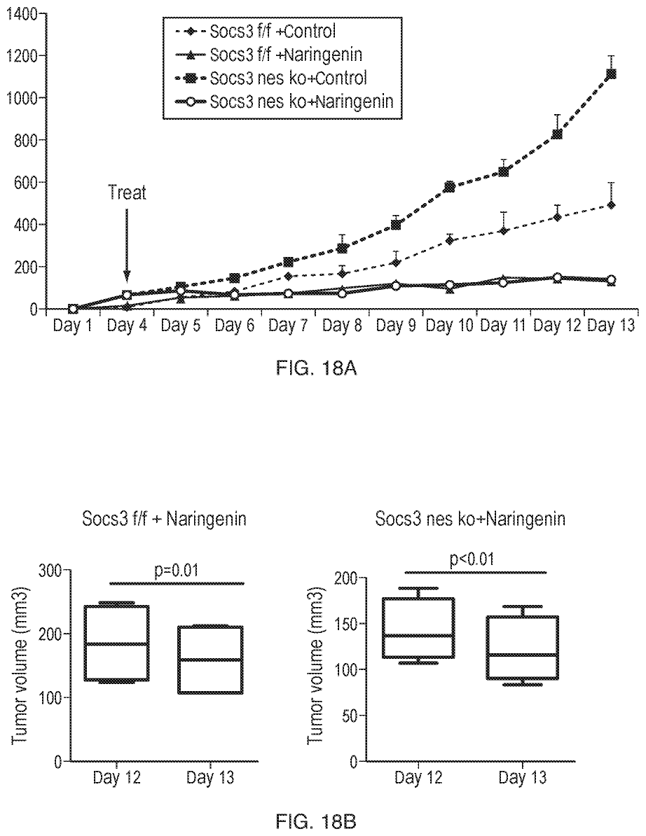

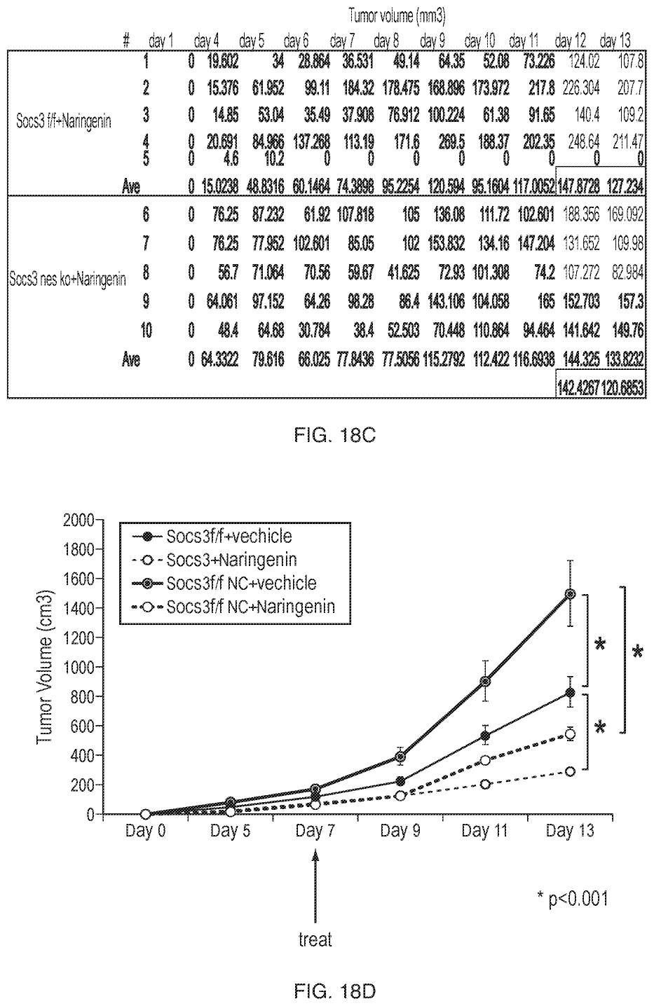

The surprising efficacy of localized Socs3 overexpression in reducing tumor growth prompted interest in identifying compounds capable of up-regulating Socs3. One candidate molecule for up-regulation of Socs3 was Naringenin, a flavanone compound. As shown in FIGS. 18A to 18D, administration of the flavanone naringenin to tumor-bearing mice resulted in reduction of tumor growth in all tested animals. In particular, naringenin administration (which increased Socs3 levels) significantly decreased tumor growth in both Socs3-containing and Socs3 nestin conditional knockout mice, when treatment was administered on day 4 (FIG. 18A). Naringenin administration was also observed to have shrunk tumors at the day 12-13 timepoint examined, with 100% of tumors of Socs3f/f mice and 60% of tumors in Socs3 nestin conditional knockout mice observed to have shrunk at day 13 of the timecourse (FIGS. 18B and 18C). As shown in FIG. 18D, administration of naringenin both increased Socs3 levels and significantly decreased tumor growth in both Socs3 wild-type and Socs3 nestin conditional knockout mice.

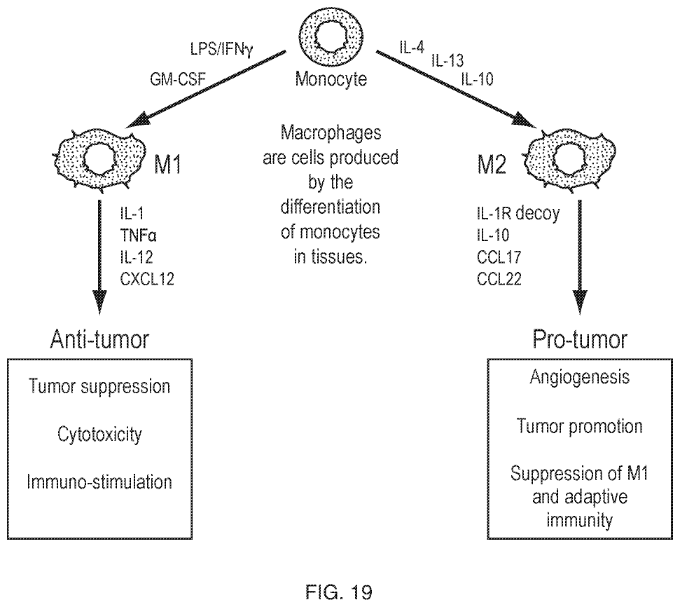

Without being bound by theory, it was posited that the molecular mechanism for the remarkable effect observed was the flavanone compound and Socs3 acting via promotion of M1 macrophages (α7AChR agonist has been described as promoting M1 macrophages, not M2 macrophages, in knockout tumor). As shown in FIG. 19, monocyte differentiation leads either to an M1 (anti-tumor, cytotoxicity, immune-stimulating) or M2 (angiogenesis, tumor promotion, suppression of M1 and adaptive immunity) outcome.

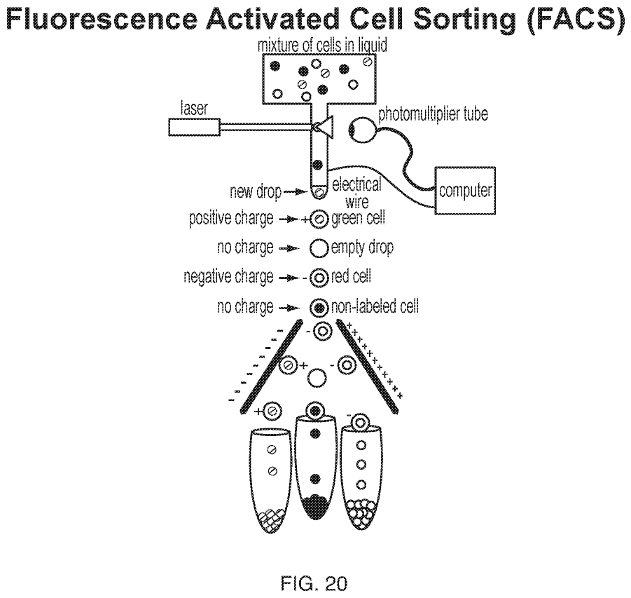

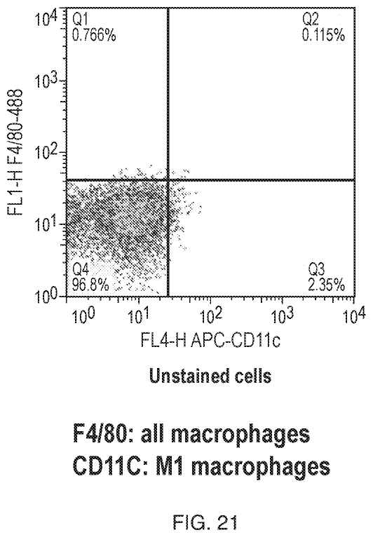

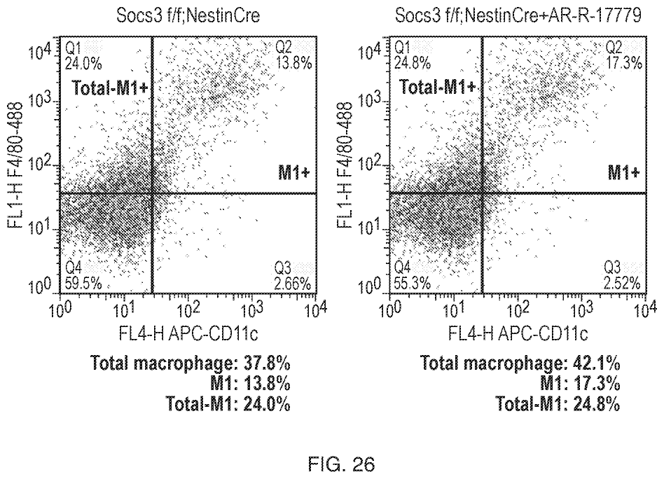

The impact of the above-recited modes of Socs3 modulation upon monocyte differentiation was examined (FIGS. 20-26), employing FACS analysis of monocyte populations (FIG. 20 provides an overview of the FACS approach, where green cells were positively charged and therefore sorted towards an anode plate while negatively charged red cells were sorted toward a cationic plate). A control FACS experiment is shown in FIG. 21, where sorting was performed upon unstained cells, resulting in essentially all cells being sorted into a single, non-fluorescent cell quadrant. Macrophages (M1 macrophages along the x-axis) were detected along both axes.

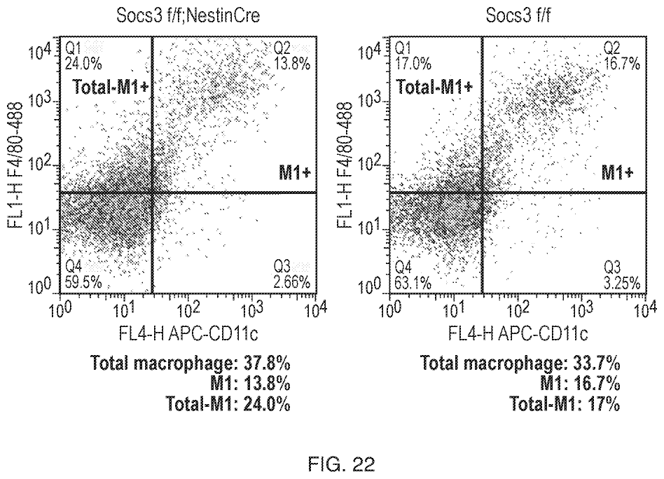

As shown in FIG. 22, nexin-conditional Socs3 neuronal deficient mice exhibited reduced relative M1 levels as compared to total macrophage levels when examined by FACS, while such Socs3 neuronal deficient mice also showed increased tumor growth. Thus, Socs3 neuronal deficiency reduced M1 macrophages, and also showed some indication of promoting M2 macrophages in tumors.

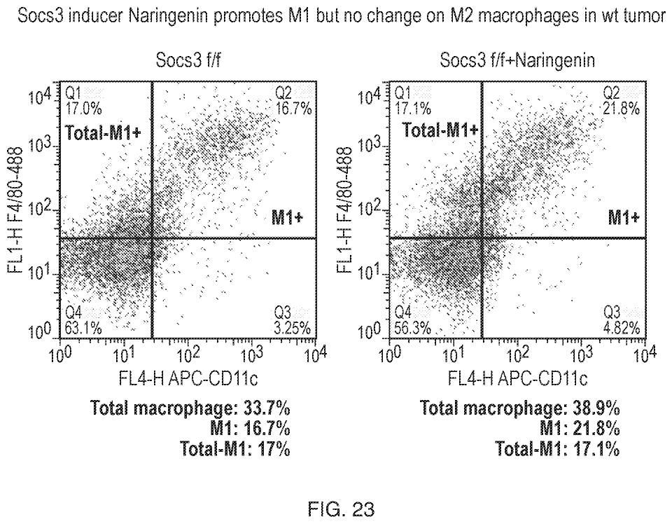

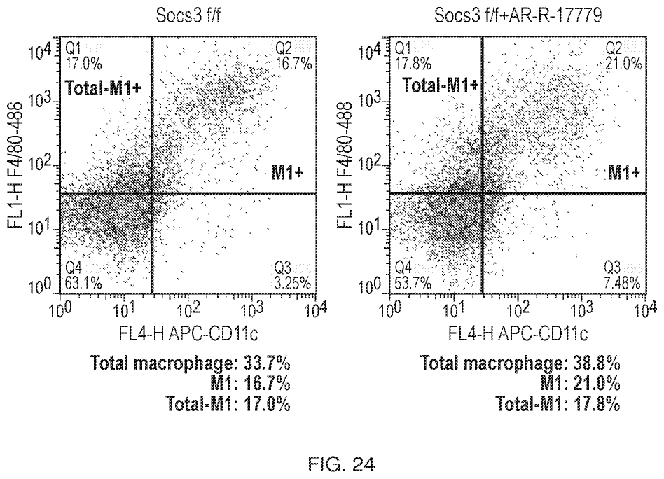

Consistent with its above-described role in increasing Socs3 levels, the Socs3 inducer Naringenin promoted M1 macrophage levels but did not change M2 macrophage levels in wild-type tumor (FIG. 23). Similarly, an α7AChR agonist also promoted M1 macrophage levels but did not change M2 macrophage levels in wild-type tumor (FIG. 24).

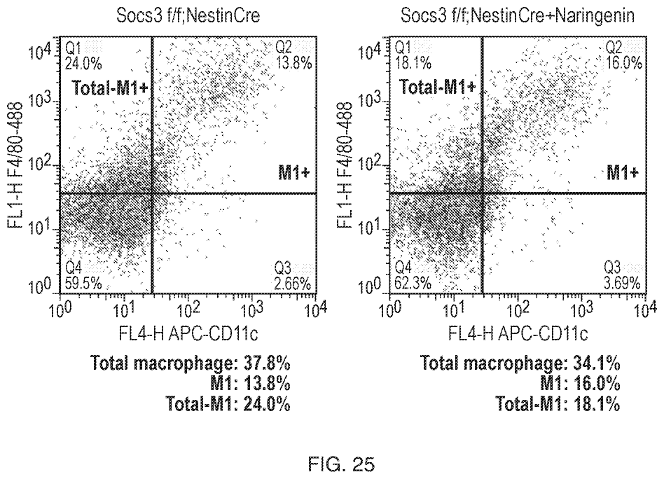

A corresponding effect of Socs3 induction and parallel effect of α7AChR agonist was also observed in tumor cells of Socs3 neuronal deficient mice. Specifically, Naringenin was observed to promote M1 macrophage levels while also reducing M2 macrophage levels in Socs3 neuronal deficient tumor (FIG. 25). Meanwhile, an α7AChR agonist promoted M1 macrophage levels while not promoting M2 macrophage levels in Socs3 neuronal deficient tumor.

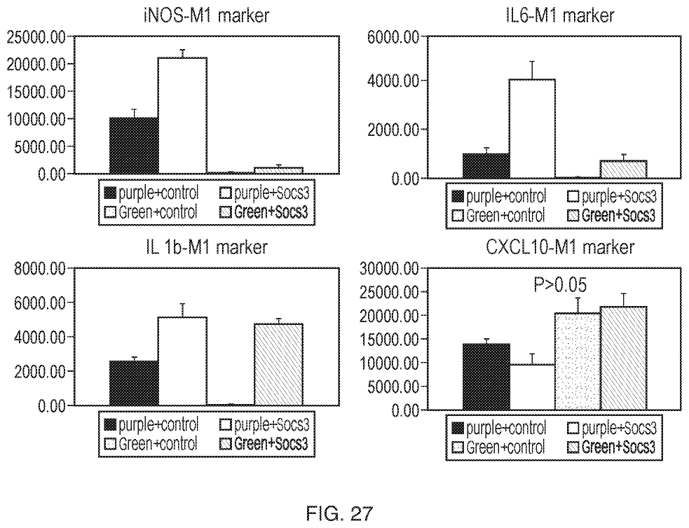

Consistent with the above FACS analyses, modulation of M1 macrophage markers was also observed when Socs3 nestin-conditional knockdown and/or lentiviral overexpression of Socs3 was performed. As shown in FIG. 27, the impact of Socs3 nestin-conditional knockdown and lentiviral overexpression of Socs3 upon levels of the M1 macrophage markers iNOS, IL 6, IL 1b and CXCL10 was examined. Notably, both Socs3 knockout and lentiviral overexpression had significant impact upon levels of iNOS, IL6 and IL 1b markers (FIG. 27).

Thus, an inducer of Socs3, the flavanone Naringenin, was identified as active for inhibiting tumor growth, via a process that appeared to involve modulation of M1 macrophage levels.

The above findings offer compelling evidence for the therapeutic role of SOCS3 in cancer models—specifically, with SOCS3 induction/overexpression acting as an inhibitor of pathological neovascularization and tumor growth. Current standard of care consists of inhibition of a single angiogenic growth factor such as VEGF, which is minimally effective at controlling tumor growth. SOCS3 biologics and/or inducers of SOCS3 can affect multiple pathways of the host pathologic vascular and neuronal response.

The ability of SOCS3 to modulate tumor growth has thus been shown with two tumor models. However, many other diseases are contemplated as targets, including retinopathy of prematurity and diabetic retinopathy.

The findings presented herein indicate an important role of host tissues, including vessels and neurons, surrounding tumors in the progression of cancer.

### Example 4: Further Demonstration that Agonists of Acetylcholine Receptor (AChR) Blocked Growth of LLC Tumor Cells and B16F10 Tumor Cells

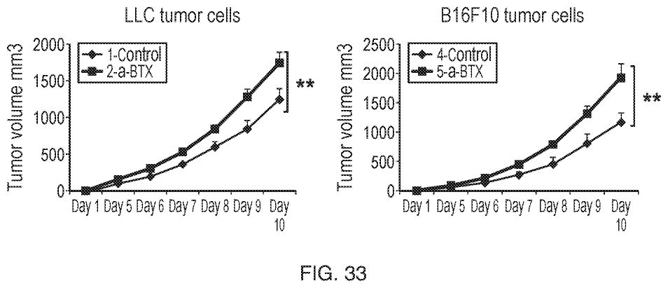

The phenotypic impact on tumor growth of modulation of acetylcholine receptor was further examined using molecular antagonists and agonists of the ACh-AChR signaling pathway. As shown in FIG. 33, alpha-bungarotoxin (a-BTX), an inhibitor of the achetylcholine receptor (AChR) was identified as having promoted tumor growth in both LLC tumor cells and in B16F10 tumor cells, when administered to model mice. Thus, it was post-synaptically demonstrated that dysfunctional motor neurons could control tumor growth.

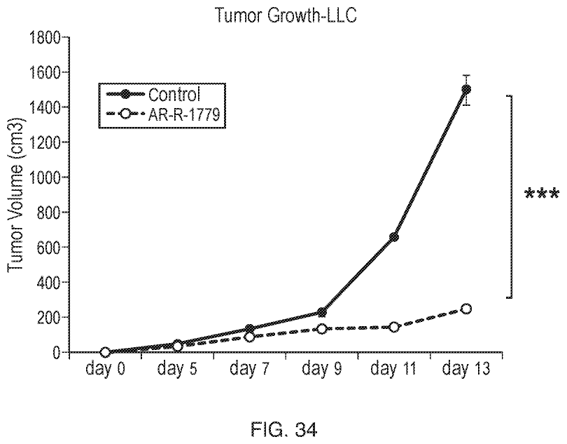

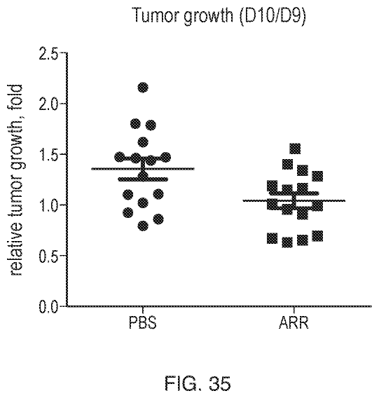

Administration of an agonist of AChR was then examined. As shown in FIG. 34, AR-R17779, an alpha-7 nicotinic AChR agonist, significantly prevented tumor growth when administered to LLC tumor cells. Consistent with observation of this effect, AR-R17779 was also identified to have decreased tumor size on day 10 post-implantation in model mice (FIG. 35).

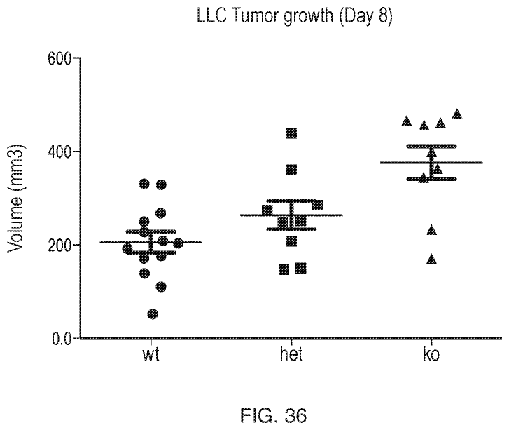

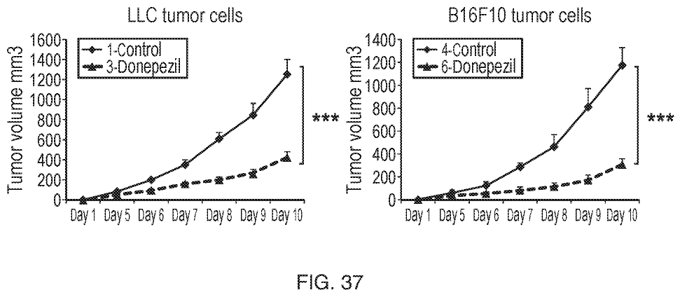

Evidence of a role for AChR in tumor growth was also identified when alpha-7 nicotinic AChR knockout mice were examined. As shown in FIG. 36, alpha-7 nicotinic AChR knockout mice exhibited LLC tumors that grew faster than in wild-type mice, indicating a role for alpha-7 nicotinic AChR in both the growth and available modes of preventing such tumors.

Similar results as those for AR-R17779 were also observed for donepezil, an inhibitor of acetylcholine esterase. Specifically, donepezil administration also prevented tumor growth in both LLC tumor cells and in B16F10 tumor cells.

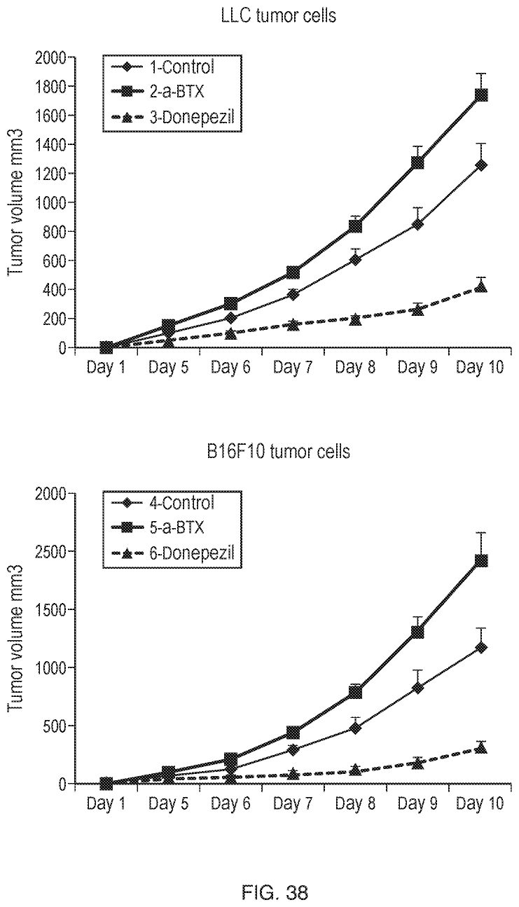

Thus, both AR-R17779 were identified as lead candidate therapeutic agents for inhibition of tumor growth in both LLC (lung carcinoma) tumor cells and in B16F10 (melanoma) tumor cells. As shown in FIG. 38, differential effects were observed for a-BTX (as an inhibitor of AChR) and donepezil (as drug promoting ACh release) in both LLC tumor cells and B16F10 tumor cells in treated mice, as compared to growth of tumors in control animals.

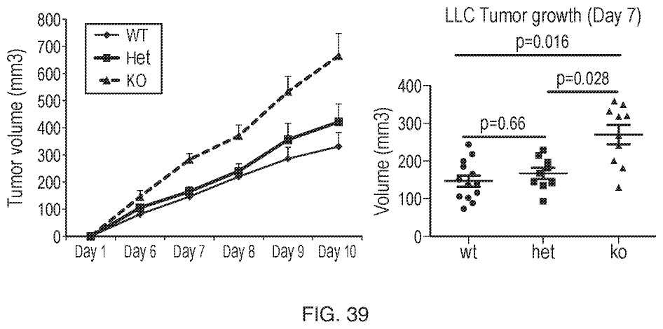

Genetic knockout approaches to reducing or eliminating alpha-7 AChR expression also resulted in apparent dose-dependent increases in rates of tumor growth (FIG. 39). Rates of LLC tumor growth were observed to be greatest in alpha-7 AChR knockout mice (where a statistically significant increase was seen), but a trend towards elevated tumor growth was also observed in even mice heterozygote for the alpha-7 AChR knockout (even though not statistically significant in the instant experiment). Thus, alpha-7 AChR was demonstrated as modulating LLC tumor reduction effects.

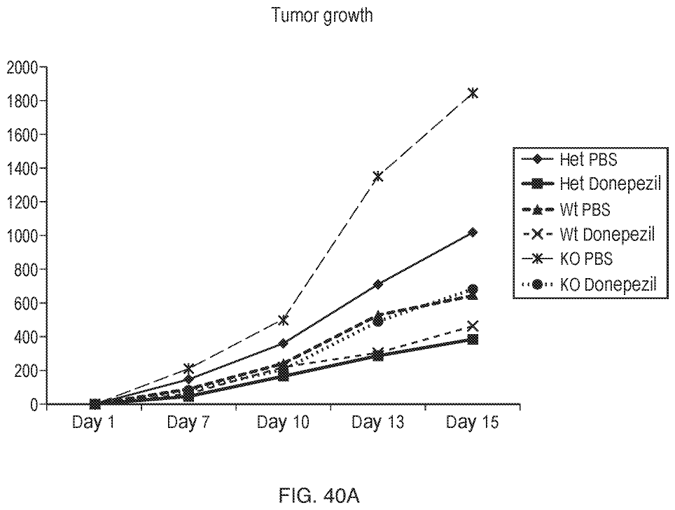

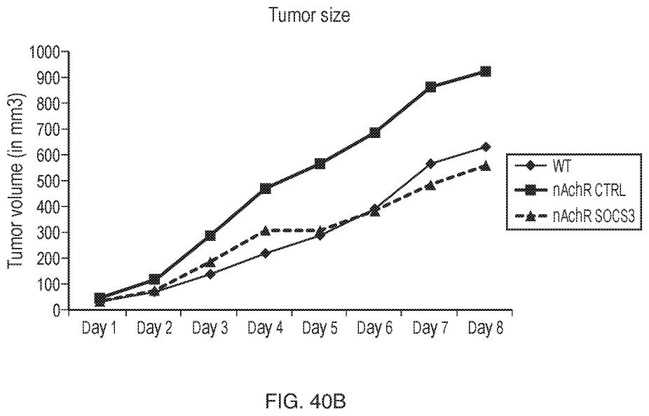

The effect of donepezil treatment was also examined in wild-type, heterozygote alpha-7 AChR knockout mice and homozygote alpha-7 AChR knockout mice (FIG. 40A). Donepezil (a general promoter of ACh release) was observed to have a dramatic impact on modulation of tumor growth rates, especially when alpha-7 AChR was inactivated, thereby confirming that alpha-7 AChR plays a large role (even if likely not the only form of AChR involved) in the ACh pathway-related modulation of tumor growth that was observed. Consistent with the trend for heterozygote mice initially observed above, treatment of even heterozygote alpha-7 AChR knockout mice with donepezil produced a significant reduction in tumor growth in such animals (as compared to PBS-treated controls). Meanwhile, a robust reduction of tumor growth rate was observed for homozygous alpha-7 AChR knockout mice treated with donepezil (as compared to PBS-treated controls).

Similar effects were also observed for lentiviral Socs3-treated mice. Specifically, it was observed that while AChR knockout promoted tumor growth, a return to wild-type levels of tumor growth was restored to AChR knockout mice when lentiviral Socs3 treatment was performed (FIG. 40B).

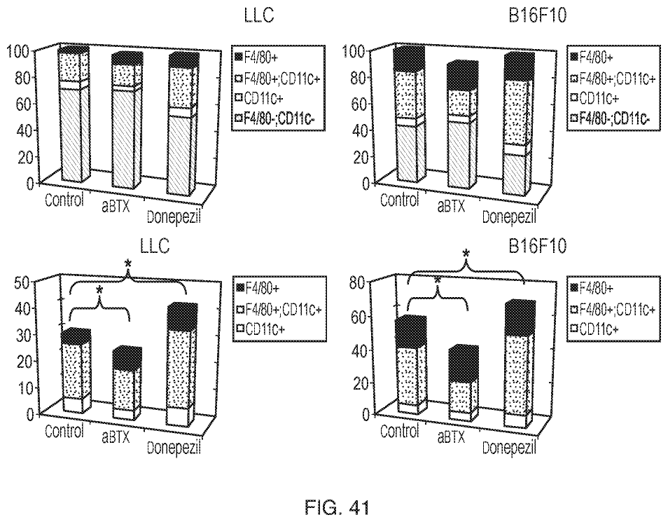

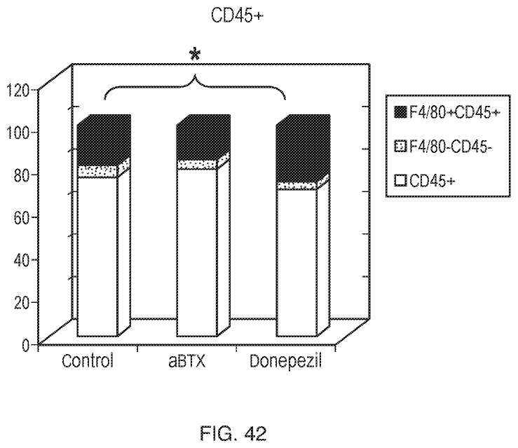

Consistent with a role for M1 macrophages in the above-identified effects, as shown in FIG. 41, anti-tumor macrophages (M1 macrophages) were also identified as increased in suppressed tumors, specifically noting the expanded population of F4/80+/CD11c+(M1 macrophage) cells in donepezil-treated samples, especially as compared to alpha-bungarotoxin (a-BTX) samples. Thus, anti-tumor M1 macrophages increased in suppressed tumors. Tumor-infiltration dendritic cells (TIDCs; macrophages) were also increased in suppressed tumors, consistent with the role of TIDCs in maintaining antitumor immunity (FIG. 42). The TIDC population is noted as CD45+; F4/80+; CD11c+ cells, which were observed to have contracted in the a-BTX-treated mice and were observed to have expanded in the donepezil-treated mice, as compared to control mice (FIG. 42).

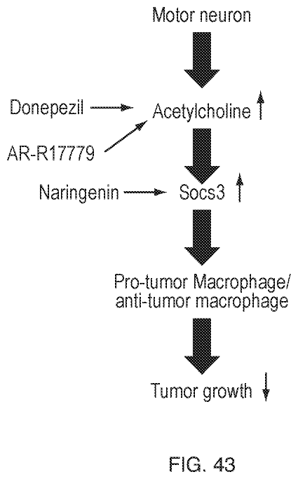

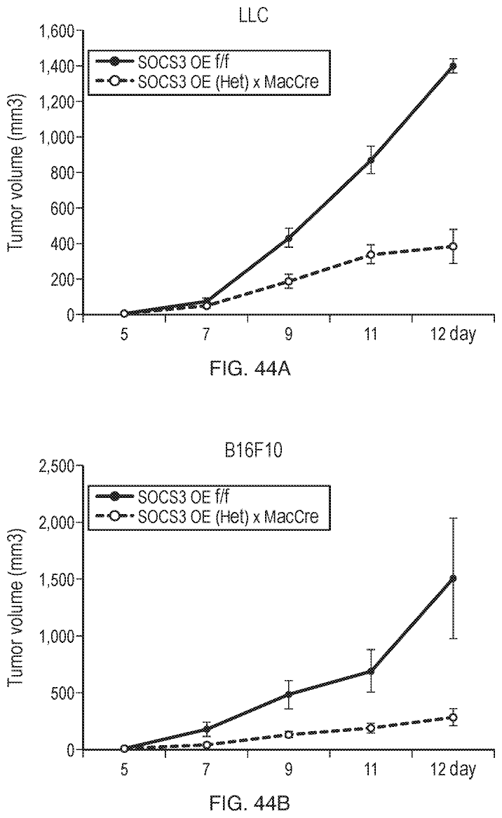

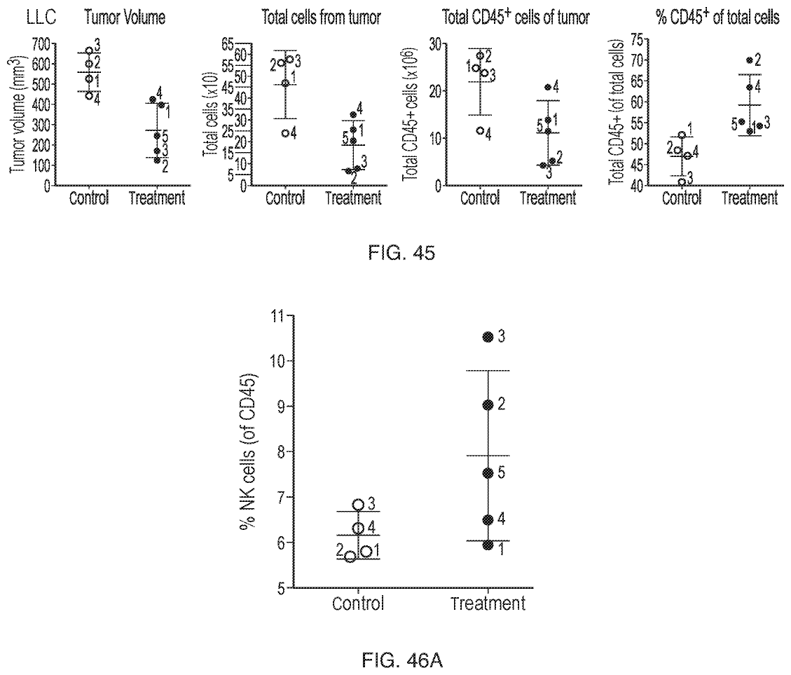

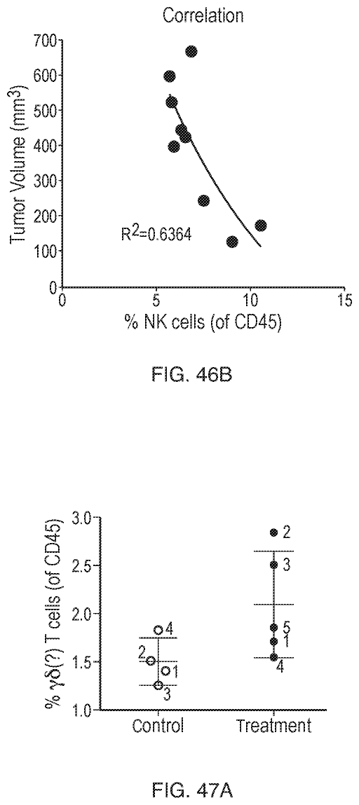

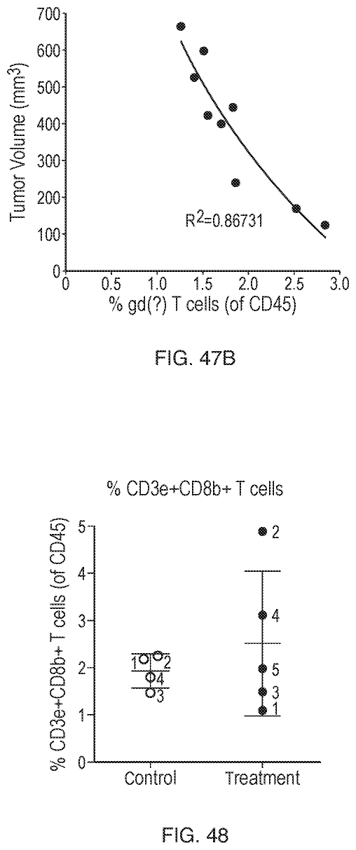

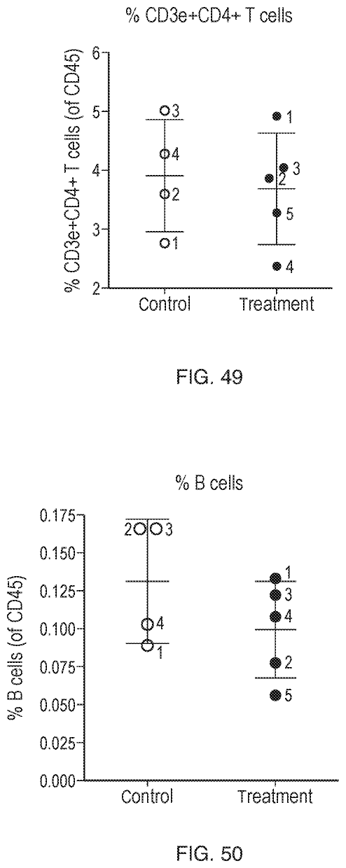

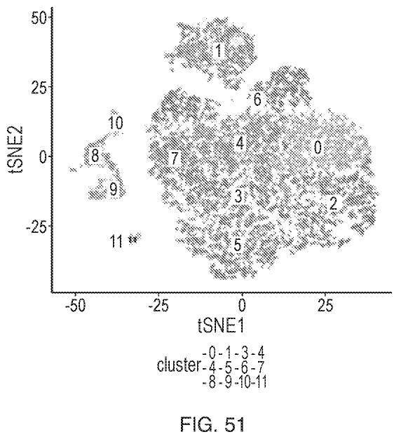

The pathway by which motor neuron control of tumor growth appears to occur via regulation of tumor-associated macrophages is accordingly diagrammed in FIG. 43.

### Example 5: Retinal Neuronal SOCS3 Governed VEGF Signaling and Neovascular Growth

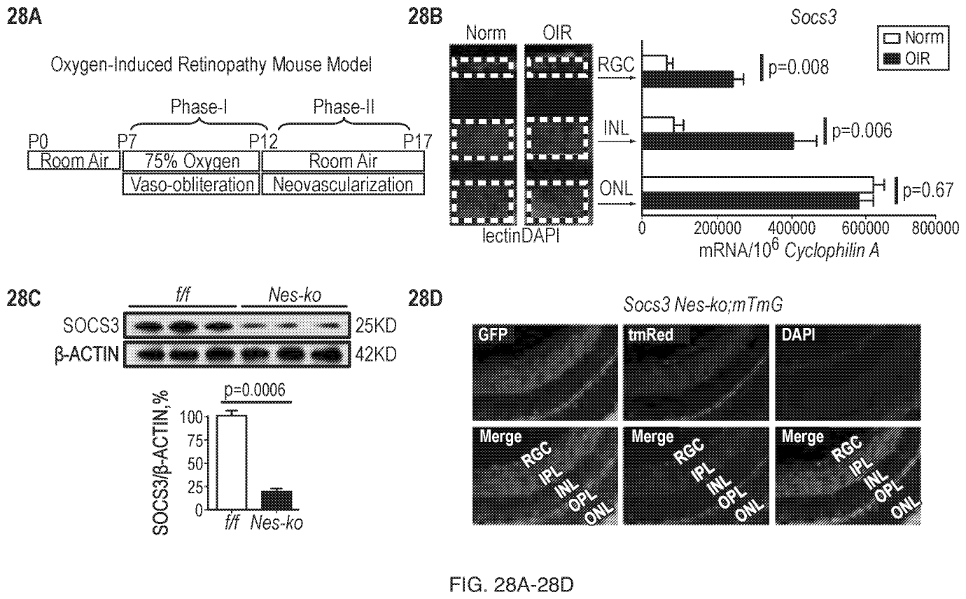

To examine the role of neuronal Socs3 in controlling pathologic retinal angiogenesis, pathological neovascularization was generated in a mouse model of oxygen-induced retinopathy (OIR) (FIG. 28A). To localize Socs3 expression in OIR mice retinas, the retinal layers were laser-capture microdissected (FIG. 28B), and each isolated layer was assessed for specific mRNA expression using qPCR. Previously, it was identified that Socs3 mRNA expression was highly upregulated in proliferative vessels in OIR (12). Here, it was observed that Socs3 mRNA expression was also highly upregulated in retinal ganglion cells (RGC) and inner nuclear layers (INL) in P17 OIR retinas, as compared with age-matched room air controls, without any change in the outer nuclear layer (ONL). Thus, Socs3 mRNA was localized in neuronal layers.

The role of neuronal/glial Socs3 in pathologic retinal angiogenesis was also investigated in OIR mice with a nestin-specific knockout of Socs3 (Socs3 Nes-ko). The nestin-Cre recombination appeared in most neural and glial cells within the retina (15-17). Decreased Socs3 retinal levels were confirmed with western blot in Socs3 Nes-ko retinas (FIG. 28C). The nestin-Cre recombination labeled with GFP was confirmed in most neural/glial cells especially in INL and RGCs (FIG. 28D) using Socs3 Nek-ko mice crossed with mTmG reporter mice. Conditional Socs3 Nes-ko OIR retinas showed ˜40% more retinal neovascularization (NV) than littermate control Socs3f/f at P17 (Socs3 Nes-ko, 11.65%±0.65%; Socs3f/f, 8.30%±0.57%; p<0.001, n=10-28) (FIGS. 29A-D, I). There was also significantly less vaso-obliterated (VO) retinal area (Socs3 Nes-ko, 13.65±1%; Socs3f/f, 16.8±0.49%, p<0.001, n=10-28; FIGS. 29E-H, J). Socs3 Nes-ko and control mice had comparable vaso-obliterated retinal area (FIGS. 29K, L and O) at P12 in OIR and normal developmental vascular growth at P7 before O₂ exposure (FIGS. 29M, N and P), which indicated that increased vascular regrowth had occurred into vaso-obliterated areas from P12 to P17. Accordingly, neuronal/glial Socs3 attenuated pathologic neovascularization in OIR.

In the OIR model, mice are exposed to 75% oxygen to induce vessel loss followed by room air from P12-17 when the retina becomes hypoxic and pathological neovascularization occurs. During the hypoxic and proliferative phase from P12-P17, VEGF is upregulated mostly in Müller cells of the inner retina and strongly contributes to pathologic neovascularization (18-20). It was confirmed that Vegfa was highly upregulated in OIR neuronal and glial cells including RGC and INL layers in OIR retinas versus age-matched normoxic controls (FIG. 30A). Next, potential neuronal/glial SOCS3 regulation of VEGF expression and its signaling pathways was explored. In P17 OIR Socs3 Nes-ko retinas, with loss of neuronal SOCS3, Vegfa expression both at the mRNA level (FIG. 30B) and protein level (FIG. 30C) were significantly increased versus Socs3f/f controls, suggesting that neuronal/glial Socs3 deficiency promotes neuronal/glial Vegfa expression in OIR retinas. Thus, neuronal/glial Socs3 deficiency elevated VEGF expression.

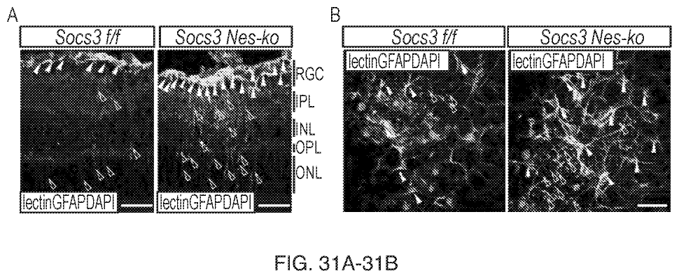

It was then investigated if Socs3 regulated other growth factors not involved in angiogenesis, specifically nerve growth factor (Ngf) expression. As shown in FIG. 30D, there was no significant difference in Ngfβ expression at P17 between Socs3 Nes-ko and Socs3f/f control OIR retinas (FIG. 30D). A previous study had demonstrated that in OIR retinas, Vegfa mRNA was localized to cell bodies identified morphologically as Müller cells (19). It was identified that Müller cells were activated in Socs3 Nes-ko retinas expressing glial fibrillary acidic protein (GFAP; FIG. 31A). In addition, astrocyte-derived VEGF was essential for hypoxia-induced neovascularization (21). Consistently, it was observed that more astrocytes were activated in the RGC layers in Socs3 Nes-ko retinas, as compared with littermate Socs3f/f controls (FIG. 31B). These data indicated that neuronal/glial Socs3 regulated Vegfa expression, potentially in Müller glial cells, astrocytes, and perhaps other neurons.

The above data indicated that neuronal/glial SOCS3 controlled in part the upregulation of VEGF, a key angiogenic protein, secreted from neuronal/glial cells, which acted on vascular endothelial cells, resulting in retinal neovascularization. It was examined if hypoxia-inducible factor 1-alpha (HIF-1α) was involved in VEGF expression in Socs3 Nes-ko OIR retinas, and it was observed that in P17 Socs3 Nes-ko OIR retinas, the expression of HIF-1a target genes, erythropoietin (Epo) and angiopoietin-like 4 (Angptl4), was at the same level as littermate Socs3f/f OIR controls (FIG. 32A). Such results indicated that HIF-1α likely primarily regulated VEGF in neuronal/glial Socs3-deficient OIR retinas. Accordingly, neuronal/glial Socs3 deficiency feedback was identified to enhanced STAT3 activation.

SOCS3 binds to both JAK kinase and interleukin-6 receptor, which was previously identified to result in the inhibition of STAT3 activation (22). Previous studies showed that STAT3 activation regulated the expression of VEGF (23, 24). STAT3 activity in response to neuronal/glial Socs3 expression level was examined, and it was observed that phospho-STAT3, the active form of STAT3, was increased 3.5 fold in P17 Socs3 Nes-ko OIR whole retinas versus Socs3f/f OIR controls (p=0.01) (FIG. 32B). These results indicated that STAT3 activation was elevated by neuronal/glial SOCS3 deficiency, which likely lead to Vegfa upregulation. Therefore, neuronal/glial Socs3 was identified as an important regulator of pathologic angiogenesis that, without wishing to be bound by theory, likely acted through STAT3-mediated VEGF expression.

It was further examined whether the VEGF signaling pathway was activated by neuronal/glial Socs3 deficiency, and specifically VEGF-induced ERK activation (25). In Socs3 Nes-ko OIR P17 retinas, phospho-ERK levels were significantly increased versus Socs3f/f OIR retinas (p=0.003) (FIG. 32C). These results indicated that neuronal/glial Socs3 suppression of pathologic endothelial activation likely acted, at least in part, by inhibiting VEGF production via ERK. Thus, neuronal/glial Socs3 deficiency was identified to have upregulated the VEGF signaling pathway.

### Example 6: Synthesis of Therapeutic Modified Socs3 Fusion Protein

The above Examples provided a compelling proof-of-principle for the therapeutic value of SOCS3 in cancer models. An even larger anti-proliferative role for SOCS3 likely exists in various pathological contexts including proliferative retinopathies, such as oxygen-induced and diabetic retinopathy.

In the current example, sdAb cross-reactive against EGFR and EGFRvIII (named EG2) is fused to SOCS3 and engineered to increase circulation half-life using at least one of the following strategies: (a & b) fusion of one or two EG2 molecules to the human Fc fragment, which is then fused to SOCS3 on the C-terminus, resulting in a mono- or bivalent construct, EG2-hFc-SOCS3, (c) or a fusion of human Fc fragment with SOCS3 (C-terminus) without EG2. These constructs are analyzed in vitro for their kinetic binding properties to EGFR and EGFRvIII and their ability to promote SOCS3 internalization in endothelial and tumor cells. Constructs are then screened ex vivo for optimal efficiency/efficacy using proliferation assays of endothelial cells, aortic sprouting assays and tumors. A targeting EG2-SOCS3 fusion that shows the best efficacy in vitro and/or ex vivo is thereby selected.

In vivo studies are performed, involving design of long plasma half-life SOCS3-EG2 fusions (i.e., EG2-Fc-SOCS3 fusion). Alternatively, the SOCS3-EG2 molecule is PEGylated to achieve extended circulating half-life. The SOCS3-EG2 fusion protein is evaluated for efficacy, potency and/or duration of effec in an appropriate model system (e.g., a xenograft model of neoplasia, mouse model of retinopathy, glioblastoma model, etc.). The SOCS3-EG2 fusion protein may be administered to the site of the tumor directly, may be generally administered, or may be use to target tumor beds and host tissues. In vivo effective SOCS3-EG2 fusion protein(s) are thereby identified.

Optionally, SOCS3 or SOCS3-EG2 is expressed in fusion with cell-penetrating peptides (TAT-like). Select peptide(s) that mediate direct penetration are fused to SOCS3 or SOCS3-EG2. Inherent leakiness of neovessels in tumor bed allow accumulation of fusion protein at tumor sites and internalization in host tissues and tumors. Such cell-penetrating protein-SOCS3 fusion proteins are tested via the above-recited in vitro, ex vivo and/or in vivo methods, thereby identifying effective cell-penetrating protein-SOCS3 fusion proteins for further study and/or development as therapeutics.

### Example 7: Identification of Therapeutic Inducers of Socs3

A SOCS3 promoter-reporter gene construct is synthesized and transfected into a mammalian cell line, which is then used for screening of test agents to identify a test agent capable of inducing SOCS3 expression and/or levels. Exemplary reporter genes include luciferase, GFP, BFP, CAT, etc. Exemplary mammalian cell lines include oncogene cell lines (e.g., melanoma, lung, etc.), neuronal (e.g., motor neuron) cell lines, vascular cell lines, muscle cell lines, etc. Cells containing the reporter gene construct are contacted with test agents/compounds (e.g., libraries of small molecules, peptides, peptide mimetics, recombinant proteins, synthetic proteins, antibodies, etc.) and reporter gene levels are monitored/measured. Reporter gene levels are compared to those of an appropriate control cell/cell line. Where elevated levels of SOCS3 are identified in the presence of test agent, a candidate inducer of SOCS3 is thereby identified. Such screening methods can be performed in parallel using multi-well plates and libraries of test compounds, thereby surveying large broad representations of compound space for agents capable of inducing SOCS3.

### Example 8: Socs3 Promotes Immune Cell Infiltration into Tumors

Socs3 can promote immune cell infiltrating into tumors to kill tumor cells. Without wishing to be bound by theory Socs3 can work together with anti-PD-1 to improve the efficacy of anti-PD1 on “cold tumors” through promoting immune cell infiltration and activation.

To confirm the immune cell types that infiltrate into tumors after modulating the level of Socs3, a drop-seq (single cell) analysis was conducted. Drop-seq data confirmed the FACS results that Socs3 controls immune cell infiltrating into tumor microenvironments (FIG. 52). The immune cells infiltrating into B16F10 tumors are mainly natural killer cells and T cells including CD8⁺ T cells. In Drop-seq data γδT cells were not seen, it could because the total γδ T cell number is too low for sequencing read out within 3000 cells (Table 1). Approximately 80% of 3000 cells were sequenced which were barcoded for analysis. Sequencing of more total cells is to be conducted to further confirm if there are γδ T cells infiltrating into tumors after Naringenin treatment.

TABLE 1

| | [symbol] 0 | [symbol] 1 | [symbol] 2 | [symbol] 3 | [symbol] 4 | [symbol] 5 | [symbol] 6 | [symbol] 7 | [symbol] 8 | [symbol] 9 | [symbol] 10 | [symbol] 11 |
|---|---|---|---|---|---|---|---|---|---|---|---|---|
| [symbol] [symbol] 1 | [symbol] | [symbol] | [symbol] | [symbol] | [symbol] | [symbol] | [symbol] | [symbol] | [symbol] | [symbol] | [symbol] | [symbol] |
| [symbol] [symbol] 2 | [symbol] | [symbol] | [symbol] | [symbol] | [symbol] | [symbol] | [symbol] | [symbol] | [symbol] | [symbol] | [symbol] | [symbol] |
| [symbol] [symbol] 3 | [symbol] | [symbol] | [symbol] | [symbol] | [symbol] | [symbol] | [symbol] | [symbol] | [symbol] | [symbol] | [symbol] | [symbol] |
| Socs3 [symbol] [symbol] 1 | [symbol] | [symbol] | [symbol] | [symbol] | [symbol] | [symbol] | [symbol] | [symbol] | [symbol] | [symbol] | [symbol] | [symbol] |
| Socs3 [symbol] [symbol] 2 | [symbol] | [symbol] | [symbol] | [symbol] | [symbol] | [symbol] | [symbol] | [symbol] | [symbol] | [symbol] | [symbol] | [symbol] |
| Socs3 [symbol] [symbol] 3 | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] |
| % | [symbol] | [symbol] | [symbol] | [symbol] | [symbol] | [symbol] | [symbol] | [symbol] | [symbol] | [symbol] | [symbol] | [symbol] |
| | up | down | down | up | up | down | up | up | down | down | down | down |

| | | | |
|---|---|---|---|
| Cell type | [symbol] | [symbol] | [symbol] |

| | | |
|---|---|---|
| [symbol] | [symbol] | T cells |
| [symbol] | [symbol] | CD8+ T cells |

[symbol]

[symbol]

[symbol]

indicates data missing or illegible when filed

### Example 9: Materials and Methods

### Animals

All animal studies were approved by the Institutional Animal Care and Use Committee at the Boston Children's Hospital. Nestin-Cre expressing C57Bl/6 mice (Jackson Laboratory, stock #003771) were crossed with Socs3 flox/flox (Socs3 f/f) mice (kind gift of Dr. A. Yoshimura) to generate Socs3 Nes-ko and littermate control flox mice. mTmG reporter mice were from the Jackson Laboratory (stock #007576). C57.1/6 mice were from the Jackson Laboratory (stock #000664).

### Oxygen-Induced Retinopathy (OIR) and Vessel Quantification

QIR was carried out in neonatal mice as described previously (14). Briefly, mouse pups with their nursing mothers were exposed to 75% oxygen from postnatal day (P) 7 to 12, then returned to room air until P17 (FIG. 28A). The retinas were collected at P17 followed by retina dissection, staining overnight with fluorescent Griffonia Simplicifolia Isolectin IB₄ (Invitrogen) and flat-mounting. The avascular (vaso-obliteration, VO) and pathologic neovascularization (NV) areas were quantified (36) by using Adobe Photoshop (Adobe Systems) and Image J (National Institutes of Health, http://imagej.nih.gov/ij/). Mice with bodyweight less than 5 grams at P17 were excluded from the study (37).

### Laser Capture Microdissection, RNA Isolation and Quantitative RT-PCR

Cross-sectional retinal layers were laser microdissected according to manufacturer's instructions (Leica LMD6000). Briefly, eyes were enucleated from C57Bl/6J wild type mice at P17 in either OIR or normoxic conditions and embedded. 8 m sections were isolated using cryostat, mounted on ribonuclease (RNase)-free polyethylene naphthalate glass slides (Leica Microsystems; Wetzlar, Germany), followed by fixation in 50% ethanol for 15 seconds, and 30 seconds in 75% ethanol, before being washed with diethyl pyrocarbonate-treated water for 15 seconds. Sections were treated with RNase inhibitor (Roche) at 25° C. for 3 minutes. Retinal layers were then laser-capture microdissected with the Leica LMD 6000 system (Leica Microsystems) and collected directly into lysis buffer from the RNeasy Micro kit (Qiagen) followed by RNA isolation. Isolated RNA from whole retinas or laser-captured retinal layers using RNeasy kit (Qiagen) was reverse transcribed with M-MLV reverse transcriptase (Invitrogen) to generate cDNA. Quantitative RT-PCR was performed using a 7300 system (Applied Biosystems) with KAPA SYBR FAST qPCR Kits (Kapa Biosystems). Cyclophilin A was used as internal control.

### Immunohistochemistry

Immunostaining in retinas was performed as previously described (Chen et al., 2013). Briefly, eyes were isolated from P17 mice with OIR, fixed and permeabilized. The flat-mounted retinas or cross sections were stained with isolectin IB₄, anti-GFAP and DAPI, and imaged using a confocal laser scanning microscope (FV1000; Olympus).

### Immunoblot

A standard immunoblotting (IB) protocol was used. Briefly, 300 mM NaCl, 0.5% NP-40, 50 mM Tris.HCl pH7.4, 0.5 mM EDTA was used to lyse the retinas. Proteinase and phosphatase inhibitor cocktails were added. The antibodies used were: anti-pERK (Cell Signaling, 4376), anti-ERK (Cell Signaling, 4695), anti-pSTAT3 (Cell Signaling, 9131S), anti-STAT3 (Cell Signaling, 9132), j-ACTIN (Sigma, A1978).

### Statistical Analysis

Results are presented as means±SEM and compared using the 2-tailed unpaired t-test. Statistical analyses were performed with GraphPad Prism (v5.0) (GraphPad Software, Inc., San Diego, Calif.). P values <0.05 were considered statistically significant.

### REFERENCES

1. Guo S, Kim W J, Lok J, Lee S R, Besancon E, Luo B H, Stins M F, Wang X, Dedhar S, and Lo E H. Neuroprotection via matrix-trophic coupling between cerebral endothelial cells and neurons. Proceedings of the National Academy of Sciences of the United States of America. 2008; 105(21):7582-7.
2. Iadecola C. Neurovascular regulation in the normal brain and in Alzheimer's disease. Nature reviews Neuroscience. 2004; 5(5):347-60.
3. Nishijima T, Piriz J, Duflot S, Fernandez A M, Gaitan G, Gomez-Pinedo U, Verdugo J M, Leroy F, Soya H, Nunez A, et al. Neuronal activity drives localized blood-brain-barrier transport of serum insulin-like growth factor-I into the CNS. Neuron. 2010; 67(5):834-46.
4. Kern T S. Interrelationships between the Retinal Neuroglia and Vasculature in Diabetes. Diabetes & metabolism journal. 2014; 38(3):163-70.
5. Nakahara T, Mori A, Kurauchi Y, Sakamoto K, and Ishii K. Neurovascular interactions in the retina: physiological and pathological roles. Journal of pharmacological sciences. 2013; 123(2):79-84.
6. Qian H, and Ripps H. Neurovascular interaction and the pathophysiology of diabetic retinopathy. Experimental diabetes research. 2011; 2011(693426.
7. Hellstrom A, Smith L E, and Dammann O. Retinopathy of prematurity. Lancet. 2013; 382(9902):1445-57.
8. Starr R, Willson T A, Viney E M, Murray L J, Rayner J R, Jenkins B J, Gonda T J, Alexander W S, Metcalf D, Nicola N A, et al. A family of cytokine-inducible inhibitors of signalling. Nature. 1997; 387(6636):917-21.
9. Lebel E, Vallieres L, and Rivest S. Selective involvement of interleukin-6 in the transcriptional activation of the suppressor of cytokine signaling-3 in the brain during systemic immune challenges. Endocrinology. 2000; 141(10):3749-63.
10. Jiang Y, Zhang Q, Soderland C, and Steinle J J. TNFalpha and SOCS3 regulate IRS-1 to increase retinal endothelial cell apoptosis. Cellular signalling. 2012; 24(5):1086-92.
11. Smith P D, Sun F, Park K K, Cai B, Wang C, Kuwako K, Martinez-Carrasco I, Connolly L, and He Z. SOCS3 deletion promotes optic nerve regeneration in vivo. Neuron. 2009; 64(5):617-23.
12. Stahl A, Joyal J S, Chen J, Sapieha P, Juan A M, Hatton C J, Pei D T, Hurst C G, Seaward M R, Krah N M, et al. SOCS3 is an endogenous inhibitor of pathologic angiogenesis. Blood. 2012; 120(14):2925-9.
13. Marine J C, McKay C, Wang D, Topham D J, Parganas E, Nakajima H, Pendeville H, Yasukawa H, Sasaki A, Yoshimura A, et al. SOCS3 is essential in the regulation of fetal liver erythropoiesis. Cell. 1999; 98(5):617-27.
14. Smith L E, Wesolowski E, McLellan A, Kostyk S K, D'Amato R, Sullivan R, and D'Amore P A. Oxygen-induced retinopathy in the mouse. Investigative ophthalmology & visual science. 1994; 35(1):101-11.
15. Arnold T D, Ferrero G M, Qiu H, Phan I T, Akhurst R J, Huang E J, and Reichardt L F. Defective retinal vascular endothelial cell development as a consequence of impaired integrin alphaVbeta8-mediated activation of transforming growth factor-beta. The Journal of neuroscience: the official journal of the Society for Neuroscience. 2012; 32(4):1197-206.
16. Michan S, Juan A M, Hurst C G, Cui Z, Evans L P, Hatton C J, Pei D T, Ju M, Sinclair D A, Smith L E, et al. Sirtuin1 over-expression does not impact retinal vascular and neuronal degeneration in a mouse model of oxygen-induced retinopathy. PloS one. 2014; 9(1):e85031.
17. Chen J, Michan S, Juan A M, Hurst C G, Hatton C J, Pei D T, Joyal J S, Evans L P, Cui Z, Stahl A, et al. Neuronal sirtuin1 mediates retinal vascular regeneration in oxygen-induced ischemic retinopathy. Angiogenesis. 2013; 16(4):985-92.
18. Alon T, Hemo I, Itin A, Pe'er J, Stone J, and Keshet E. Vascular endothelial growth factor acts as a survival factor for newly formed retinal vessels and has implications for retinopathy of prematurity. Nature medicine. 1995; 1(10):1024-8.
19. Pierce E A, Avery R L, Foley E D, Aiello L P, and Smith L E. Vascular endothelial growth factor/vascular permeability factor expression in a mouse model of retinal neovascularization. Proceedings of the National Academy of Sciences of the United States of America. 1995; 92(3):905-9.
20. Stone J, Chan-Ling T, Pe'er J, Itin A, Gnessin H, and Keshet E. Roles of vascular endothelial growth factor and astrocyte degeneration in the genesis of retinopathy of prematurity. Investigative ophthalmology & visual science. 1996; 37(2):290-9.
21. Weidemann A, Krohne T U, Aguilar E, Kurihara T, Takeda N, Dorrell M I, Simon M C, Haase V H, Friedlander M, and Johnson R S. Astrocyte hypoxic response is essential for pathological but not developmental angiogenesis of the retina. Glia. 2010; 58(10):1177-85.
22. Wei X, Wang G, Li W, Hu X, Huang Q, Xu K, Lou W, Wu J, Liang C, Lou Q, et al. Activation of the JAK-STAT3 pathway is associated with the growth of colorectal carcinoma cells. Oncology reports. 2014; 31(1):335-41.
23. Wei D, Le X, Zheng L, Wang L, Frey J A, Gao A C, Peng Z, Huang S, Xiong H Q, Abbruzzese J L, et al. Stat3 activation regulates the expression of vascular endothelial growth factor and human pancreatic cancer angiogenesis and metastasis. Oncogene. 2003; 22(3):319-29.
24. Niu G, Wright K L, Huang M, Song L, Haura E, Turkson J, Zhang S, Wang T, Sinibaldi D, Coppola D, et al. Constitutive Stat3 activity up-regulates VEGF expression and tumor angiogenesis. Oncogene. 2002; 21(13):2000-8.
25. Kuriyama M, Taniguchi T, Shirai Y, Sasaki A, Yoshimura A, and Saito N. Activation and translocation of PKCdelta is necessary for VEGF-induced ERK activation through KDR in HEK293T cells. Biochemical and biophysical research communications. 2004; 325(3):843-51.
26. Stahl A, Connor K M, Sapieha P, Chen J, Dennison R J, Krah N M, Seaward M R, Willett K L, Aderman C M, Guerin K I, et al. The mouse retina as an angiogenesis model. Investigative ophthalmology & visual science. 2010; 51(6):2813-26.
27. Ozaki H, Yu A Y, Della N, Ozaki K, Luna J D, Yamada H, Hackett S F, Okamoto N, Zack D J, Semenza G L, et al. Hypoxia inducible factor-1alpha is increased in ischemic retina: temporal and spatial correlation with VEGF expression. Investigative ophthalmology & visual science. 1999; 40(1):182-9.
28. Ferrara N, Gerber H P, and LeCouter J. The biology of VEGF and its receptors. Nature medicine. 2003; 9(6):669-76.
29. Forsythe J A, Jiang B H, Iyer N V, Agani F, Leung S W, Koos R D, and Semenza G L. Activation of vascular endothelial growth factor gene transcription by hypoxia-inducible factor 1. Molecular and cellular biology. 1996; 16(9):4604-13.
30. Kallio P J, Okamoto K, O'Brien S, Carrero P, Makino Y, Tanaka H, and Poellinger L. Signal transduction in hypoxic cells: inducible nuclear translocation and recruitment of the CBP/p300 coactivator by the hypoxia-inducible factor-1alpha. The EMBO journal. 1998; 17(22):6573-86.
31. Jiang B H, Rue E, Wang G L, Roe R, and Semenza G L. Dimerization, DNA binding, and transactivation properties of hypoxia-inducible factor 1. The Journal of biological chemistry. 1996; 271(30):17771-8.
32. Chen J, Connor K M, Aderman C M, and Smith L E. Erythropoietin deficiency decreases vascular stability in mice. The Journal of clinical investigation. 2008; 118(2):526-33.
33. Xin X, Rodrigues M, Umapathi M, Kashiwabuchi F, Ma T, Babapoor-Farrokhran S, Wang S, Hu J, Bhutto I, Welsbie D S, et al. Hypoxic retinal Muller cells promote vascular permeability by HIF-1-dependent up-regulation of angiopoietin-like 4. Proceedings of the National Academy of Sciences of the United States of America. 2013; 110(36):E3425-34.
34. Perdiguero E G, Galaup A, Durand M, Teillon J, Philippe J, Valenzuela D M, Murphy A J, Yancopoulos G D, Thurston G, and Germain S. Alteration of developmental and pathological retinal angiogenesis in angptl4-deficient mice. The Journal of biological chemistry. 2011; 286(42):36841-51.
35. Pawlus M R, Wang L, and Hu C J. STAT3 and HIF1alpha cooperatively activate HIF1 target genes in MDA-MB-231 and RCC4 cells. Oncogene. 2014; 33(13):1670-9.
36. Connor K M, Krah N M, Dennison R J, Aderman C M, Chen J, Guerin K I, Sapieha P, Stahl A, Willett K L, and Smith L E. Quantification of oxygen-induced retinopathy in the mouse: a model of vessel loss, vessel regrowth and pathological angiogenesis. Nat Protoc. 2009; 4(11):1565-73.
37. Stahl A, Chen J, Sapieha P, Seaward M R, Krah N M, Dennison R J, Favazza T, Bucher F, Lofqvist C, Ong H, et al. Postnatal weight gain modifies severity and functional outcome of oxygen-induced proliferative retinopathy. The American journal of pathology. 2010; 177(6):2715-23.

What is claimed is:

1. A method of inhibiting pathological blood vessel growth comprising:
administering to a subject with pathological blood vessel growth a modified suppressor of cytokine signaling (SOCS3) fusion protein, a SOCS3 peptidomimetic or a vector expressing a SOCS3 polypeptide,
thereby inhibiting pathological blood vessel growth.

2. A method of inhibiting pathological blood vessel growth comprising:
administering a flavanone to a subject with pathological blood vessel growth, thereby inhibiting pathological blood vessel growth.

3. A method for inhibiting the growth of a tumor in a subject comprising:
administering a flavanone to the subject in an amount sufficient to increase the level of SOCS3 in said tumor, in a tumor-associated tissue, or in the general host tissue of said subject,
thereby inhibiting the growth of said tumor in said subject.

4. A method for inhibiting the growth of a tumor in a subject comprising:
administering a modified SOCS3 fusion protein or a vector that expresses a modified SOCS3 fusion protein or a SOCS3 peptidomimetic to the subject in an amount sufficient to increase the level of SOCS3 in said tumor, in a tumor-associated tissue, or host tissue of said subject,
thereby inhibiting the growth of said tumor in said subject.

5. The method of claim 2 or 3, wherein said flavanone is selected from the group consisting of Butin, Eriodictyol, Hesperetin, Hesperidin, Homoeriodictyol, Isosakuranetin, Naringenin, Naringin, Pinocembrin, Poncirin, Sakuranetin, Sakuranin and Sterubin.

6. The method of claim 3 or 4, wherein the tumor is a solid tumor.

7. The method of claim 6, wherein said solid tumor is selected from the group consisting of melanoma, lung cancer, gastric cancer, liver cancer, colon cancer, esophageal cancer, and pancreatic cancer.

8. The method of any one of claims 3, 4, 6 or 7, wherein said tumor-associated tissue is a host tumor bed, vascular tissue, neural tissue, or muscle tissue.

9. The method of claim 3 or 4, wherein the host tissue or general host tissue is stromal tissue of the subject.

10. The method of claim 4, wherein the vector that expresses a modified SOCS3 fusion protein is a viral vector, optionally an AAV or lentiviral vector.

11. The method of claim 4, wherein the vector is an adenoviral vector, optionally an AAV vector selected from the group consisting of AAV-1 to AAV-9.

12. The method of claim 4, wherein expression of the viral vector is tissue-specific.

13. The method of claim 4, wherein expression of the viral vector is global.

14. An in vitro method for identifying a candidate inducer of SOCS3 protein expression comprising:
making a SOCS3 reporter gene construct comprising a reporter gene under control of the SOCS3 promoter;
introducing the SOCS3 reporter gene construct into a mammalian cell;
contacting the mammalian cell with a test agent under conditions suitable for SOCS3 reporter gene expression in said mammalian cell; and
comparing levels of the SOCS3 reporter gene in said mammalian cell contacted with said test agent with levels of the SOCS3 reporter gene in an appropriate control mammalian cell,
wherein identification of elevated SOCS3 reporter gene levels in said mammalian cell contacted with the test agent identifies the test agent as a candidate inducer of SOCS3 protein expression.

15. The method of claim 14, wherein the mammalian cell contacted with the test agent is selected from the group consisting of a carcinoma cell, a neuronal cell, an immune cell, a macrophage, a vascular cell and a muscle cell.

16. A method for inhibiting the growth of a tumor in a subject comprising:
administering an inducer of SOCS3 protein identified by the method of claim 14 to the subject in an amount sufficient to increase the level of SOCS3 in said tumor or in a tumor-associated tissue of the subject,
thereby inhibiting the growth of the tumor in said subject.

17. A composition comprising a modified suppressor of cytokine signaling (SOCS3) protein.

18. The composition of claim 17 wherein the protein is active intracellularly.

19. The composition of claim 17 or 18, wherein the modified SOCS3 protein is fused to an antibody, or fragment thereof.

20. The composition of claim 19, wherein the antibody, or fragment thereof, is a single chain antibody (scFv).

21. The composition of claim 20, wherein the scFv is a cell-internalizing scFv.

22. The composition of claim 21, wherein the scFv is internalized in pathologic blood vessels, tumor associated cells or neurons.

23. The composition of any one of claims 17 through 21, wherein the antibody, or fragment thereof, is a single domain antibody (sdAb).

24. The composition of claim 23, wherein the sdAb is bispecific.

25. The composition of any one of claims 17 through 24, wherein the modified SOCS3 protein is fused to a cell-penetrating peptide.

26. The composition of any one of claims 17 through 25 further comprising one or more molecules to increase the half-life.

27. A fusion protein comprising a modified SOCS3 protein fused to at least one scFv.

28. The fusion protein of claim 27, wherein the scFv is a cell-internalizing scFv.

29. The fusion protein of claim 28, wherein the scFv is internalized in pathologic blood vessels or neurons.

30. A fusion protein comprising a modified SOCS3 protein fused to at least one sdAb.

31. A fusion protein comprising a modified SOCS3 protein fused to at least one cell-penetrating peptide.

32. The fusion protein of any one of claims 27 through 31, further comprising one or more molecules to increase half-life.

33. A method of treating an autoimmune disease or sepsis comprising:
administering to a subject with an autoimmune disease or sepsis a composition comprising a flavanone, a candidate inducer of SOCS3 protein identified by the method of claim 14, a modified SOCS3 fusion protein, or a vector that expresses a modified SOCS3 fusion protein,
thereby treating the autoimmune disease or sepsis.

34. The method of claim 33, wherein the autoimmune disease is associated with pathological blood vessel growth.

35. The method of claim 33 or 34, wherein the composition is administered to a host tissue.

36. The method of any one of claims 33 through 35, wherein the autoimmune disease is retinopathy.

37. The method of claim 36, wherein the retinopathy is retinopathy of prematurity, diabetic retinopathy or age related macular degeneration.

38. The method any one of claims 33 through 35, wherein the autoimmune disease is juvenile rheumatoid arthritis (JRA).

39. The method of claim 33, wherein the subject has sepsis.

40. A method of inhibiting tumor growth comprising:
administering to a subject with a solid tumor a composition comprising a candidate inducer of SOCS3 protein identified by the method of claim 14, a modified SOCS3 fusion protein, a vector that expresses a SOCS3 polypeptide, or a flavanone
thereby inhibiting tumor growth.

41. The method of claim 40, wherein the composition is administered to the tumor, and/or optionally to tumor-associated cells, optionally the cells are associated with the tumors.

42. The method of claim 41, wherein the composition is administered to nerve fibers.

43. The method of claim 42, wherein the nerve fibers are associated with the tumors.

44. A kit comprising the modified suppressor of cytokine signaling SOCS3 protein of claim 17 or a vector that expresses a SOCS3 polypeptide, and instructions for its use in inhibiting pathological blood vessel growth or pathological neovascularization.

45. A kit comprising the modified suppressor of cytokine signaling SOCS3 protein of claim 17 or a vector that expresses a SOCS3 polypeptide, and instructions for its use in inhibiting tumor growth.

46. A method of inhibiting or decreasing solid tumor in a subject, said method comprising administering an effective amount of SOCS3, a vector that expresses a SOCS3 polypeptide, and/or a SOCS3 inducing agent to a tumor-associated tissue in the subject and/or to a host tissue of the subject, such that growth of the solid tumor is inhibited or decreased.

47. A method of inhibiting or decreasing solid tumor growth in a subject, said method comprising
selecting a subject having a solid growth tumor and
systemically administering an effective amount of SOCS3, a vector that expresses a SOCS3 polypeptide, and/or a SOCS3 inducing agent to the subject,
such that growth of the solid tumor is inhibited or decreased.

48. The method of claim 46 or 47, wherein the solid tumor is selected from the group consisting of a lung carcinoma, a glioblastoma, a gastric adenocarcinoma, a hepatocellcular carcinoma, and a melanoma.

49. The method of any one of claims 46 through 48, further comprising delivering the SOCS3 and/or the SOCS3 inducing agent directly to the solid tumor.

50. The method of any one of claims 46 through 49, wherein the subject does not have a side effect from the method, wherein the side effect is selected from the group consisting of substantial hair loss, gastrointestinal bleeding, and chemo brain.

51. A method of preventing tumor formation in a subject predisposed to a malignancy or having pre-cancer, said method comprising administering an effective amount of SOCS3, a vector that expresses a SOCS3 polypeptide, and/or a SOCS3 inducing agent to the subject, such that tumor formation is prevented.

52. The method of claim 51, wherein the subject predisposed to a malignancy has familial adenomatous polyposis or is a carrier for a BRCA1 or BRCA2 mutation associated with cancer.

53. The method of any one of claims 46 through 52, wherein the SOCS3 inducing agent is a flavanone.

54. The method of claim 53, wherein the flavanone is selected from the group consisting of Butin, Eriodictyol, Hesperetin, Hesperidin, Homoeriodictyol, Isosakuranetin, Naringenin, Naringin, Pinocembrin, Poncirin, Sakuranetin, Sakuranin and Sterubin.

55. The method of any one of claims 46 through 52, wherein the SOCS3 inducing agent is an antibody, or an antigen-binding portion thereof.

56. The method of any one of claims 46 to 52, wherein SOCS3 is either a nucleic acid encoding SOCS3 protein, or a functional fragment thereof, or a SOCS3 protein, or a functional fragment thereof.

57. The method of claim 56, wherein the nucleic acid is a viral vector.

58. The method of any one of claims 46 through 52, wherein the SOCS3 inducing agent is a modified SOCS3 fusion protein.

59. The method of any one of claims 46 through 58, wherein the SOCS3, vector that expresses a SOCS3 polypeptide, and/or a SOCS3 inducing agent is administered to the subject via a method selected from the group consisting of systemic administration, oral administration, enteral administration, and topical administration.

60. A method of inhibiting or decreasing a tumor in a subject, said method comprising administering an effective amount of an agent that promotes acetylcholine release to a tumor-associated tissue in the subject and/or to a host tissue of the subject, such that growth of the tumor is inhibited or decreased.

61. A method of inhibiting or decreasing solid tumor growth in a subject, said method comprising
selecting a subject having a solid growth tumor and
systemically administering an effective amount of an agent that promotes acetylcholine release to the subject,
such that growth of the solid tumor is inhibited or decreased.

62. The method of claim 60 or 61, wherein the tumor is selected from the group consisting of a lung carcinoma, a glioblastoma, a gastric adenocarcinoma, a hepatocellcular carcinoma, and a melanoma.

63. The method of any one of claims 60 through 62, further comprising delivering the agent that promotes acetylcholine release directly to the tumor.

64. The method of any one of claims 60 through 63, wherein the subject does not have a side effect from the method, wherein the side effect is selected from the group consisting of substantial hair loss, gastrointestinal bleeding, and chemo brain.

65. A method of preventing tumor formation in a subject predisposed to a malignancy or having pre-cancer, said method comprising administering an effective amount of an agent that promotes acetylcholine release to the subject, such that tumor formation is prevented.

66. The method of claim 66, wherein the subject predisposed to a malignancy has familial adenomatous polyposis or is a carrier for a BRCA1 or BRCA2 mutation associated with cancer.

67. The method of any one of claims 60 through 66, wherein the agent that promotes acetylcholine release is selected from the group consisting of AR-R 17779 hydrochloride; 4BP-TQS; A 582941; A 844606; 3-Bromocytisine; DMAB-anabaseine dihydrochloride; GTS 21 dihydrochloride; PHA 543613 hydrochloride; PHA 568487; PNU 282987; S 24795; SEN 12333; TC 1698 dihydrochloride; A 85380 dihydrochloride; 3-Bromocytisine; CC4; 5-Iodo-A-85380 dihydrochloride; (−)-Nicotine ditartrate; 3-pyr-Cytisine; RJR 2403 oxalate; SIB 1508Y maleate; TC 2559 difumarate; Varenicline tartrate; A 844606; A 85380 dihydrochloride; 4-Acetyl-1,1-dimethylpiperazinium iodide; 1-Acetyl-4-methylpiperazine hydrochloride; (+)-Anabasine hydrochloride; (±)-Anatoxin A fumarate; 3-Bromocytisine; Carbamoylcholine chloride; CC4; Cisapride; (−)-Cytisine; DMAB-anabaseine dihydrochloride; (±)-Epibatidine; (−)-Lobeline hydrochloride; RJR 2429 dihydrochloride; Sazetidine A dihydrochloride; SIB 1553A hydrochloride; Tropisetron hydrochloride; UB 165 fumarate; Donepezil hydrochloride; Ambenonium dichloride; Galanthamine hydrobromide; PE 154; Phenserine; Physostigmine hemisulfate; Rivastigmine tartrate; and Tacrine hydrochloride

68. The method of any one of claims 60 through 66, wherein the agent that promotes acetylcholine release to the subject is administered to the subject via a method selected from the group consisting of systemic administration, oral administration, enteral administration, and topical administration, optionally wherein the agent that promotes acetylcholine release is administered to nerve fibers, optionally nerve fibers associated with the tumor.

69. The method of any of claims 60 through 67, wherein the tumor is a solid tumor.

70. The method of claim 69, wherein said solid tumor is selected from the group consisting of melanoma, lung cancer, gastric cancer, liver cancer, colon cancer, esophageal cancer, and pancreatic cancer.

71. The method of claim 60, wherein the tumor-associated tissue is a host tumor bed, a macrophage population, a vascular tissue, a neural tissue, or a muscle tissue.

72. A method of inhibiting pathological blood vessel growth comprising:
administering to a subject with pathological blood vessel growth an agent that promotes acetylcholine release,
thereby inhibiting pathological blood vessel growth.

73. A method of inhibiting pathological blood vessel growth comprising:
administering an agent that promotes acetylcholine release to a subject with pathological blood vessel growth,
thereby inhibiting pathological blood vessel growth.

74. A method of treating an autoimmune disease or sepsis comprising:
administering to a subject with an autoimmune disease or sepsis an agent that promotes acetylcholine release,
thereby treating the autoimmune disease or sepsis.

75. The method of claim 74, wherein the autoimmune disease is associated with pathological blood vessel growth.

76. The method of claim 74 or 75, wherein the agent is administered to a host tissue.

77. The method of any one of claims 74 through 76, wherein the autoimmune disease is retinopathy.

78. The method of claim 77, wherein the retinopathy is retinopathy of prematurity, diabetic retinopathy or age related macular degeneration.

79. The method any one of claims 74 through 78, wherein the autoimmune disease is juvenile rheumatoid arthritis (JRA).

80. The method of claim 74, wherein the subject has sepsis.

81. A kit comprising an agent that promotes acetylcholine release, and instructions for its use in inhibiting pathological blood vessel growth or pathological neovascularization.

82. A kit comprising an agent that promotes acetylcholine release, and instructions for its use in inhibiting tumor growth.

83. A method of inducing immune cell infiltration of a tumor, comprising, administering to a subject an effective amount of a suppressor of cytokine signaling (SOCS3) fusion protein SOCS3, a vector that expresses a SOCS3 polypeptide, a SOCS3 peptidomimetic, a flavanone and/or a SOCS3 inducing agent to a tumor-associated tissue in the subject and/or to a host tissue of the subject, such that growth of the solid tumor is inhibited or decreased.

84. The method of claim 83, wherein induction of immune cell infiltration decreases tumor size and tumor volume

85. The method of claim 83 or 84 wherein the immune cells are cytolytic or induce cytolysis of the tumor.

85. The method of any one of claims 83 through 85 further comprising administration of one or more checkpoint blockade immunotherapeutic agents.

86. The method of claim 85, wherein the checkpoint blockade immunotherapeutic agents are specific for PD-1, PD-L1, PD-L2, CTLA-4, CD28, CD80, CD86, B7-H3, B7-H4, B7-H5, ICOS-L, ICOS, BTLA, CD137L, CD137, HVEM, KIR, 4-1BB, OX40L, CD70, CD27, CD47, CIS, OX40, GITR, IDO, TIM3, GAL9, VISTA, CD155, TIGIT, LIGHT, LAIR-1, Siglecs, A2aR or combinations thereof.

87. The method of claim 86, wherein the checkpoint blockade immunotherapeutic agent is an anti-PD-1 or anti-PD-L1.

88. The method of any one of claims 83 through 88 wherein the SOCS3 peptidomimetic comprises SEQ ID NOS: 1 or 2.


SEQUENCE LISTING (2 sequences)

SEQ ID NO: 1 - protein, 12 aa, Artificial Sequence
  Description of Artificial Sequence Synthetic peptide
LKTFSSKSEYQL

SEQ ID NO: 2 - protein, 16 aa, Artificial Sequence
  Description of Artificial Sequence Synthetic peptide
EYQLVVNAVRKLQESG